(19) United States
(12) Patent Application Publication
(10) Pub. No.: US 2022/0226480 A1
(43) Pub. Date: Jul. 21, 2022

(54) LIPID CONJUGATE PREPARED FROM SCAFFOLD MOIETY

(71) Applicant: Integrated Nanotherapeutics Inc., Vancouver, B.C. (CA)

(72) Inventors: Sam Chen, Vancouver, B.C. (CA); Yuen Yi Tam, Vancouver, B.C. (CA); Joshua Zaifman, Vancouver, B.C. (CA); Marco Ciufolini, Vancouver, B.C. (CA)

(21) Appl. No.: 17/435,384

(22) PCT Filed: Mar. 23, 2020
(86) PCT No.: PCT/CA2020/000039

§ 371 Date: Aug. 31, 2021

Related U.S. Application Data
(60) Provisional application No. 62/822,226, filed on Mar. 22, 2019.

(51) Int. Cl. A61K 47/54; A61K 47/69

(52) U.S. Cl. CPC A61K 47/543; A61K 47/6929; A61K 47/542; A61K 47/6911

(57) ABSTRACT

The application relates to a lipid conjugate of formula M-X1-L wherein M is a molecule of interest such as a drug moiety; X1 is a linker group such as ester, ether or carbamate; and L is a lipid scaffold represented by formula (IId): -L1-[L2(H)(X2R)]n-L3-[L4(H)(X2R)]p-L5-L6 and wherein L comprises 5 to 40 carbon atoms and 0 to 2 carbon-carbon double bonds. The lipid conjugate can p be formulated in a drug delivery vehicle such as a lipid nanoparticle (LNP).

### TECHNICAL FIELD

Provided herein are lipid conjugates, formulations of lipid conjugates and precursor molecules for preparing such conjugates.

### BACKGROUND

Many drugs have the potential to cure cancers, autoimmune diseases and other disorders, but their therapeutic effects are often unrealized due to their failure to reach a disease site. For example, when a drug is administered intravenously, frequently only small amounts (e.g., often less than 0.1%) of the drug actually arrives at its target. The remainder of the drug distributes throughout the rest of the body, leading to reduced therapeutic efficacy, as well as undesirable side effects.

Drug delivery systems, including lipid nanoparticles (LNP) and polymer-based vehicles have the potential to overcome this problem. The aim of such systems is to encapsulate drugs and target them specifically to parts of the body requiring therapy, such as a tumour site or a region of inflammation. This effect can be achieved by exploiting the leaky vasculature and impaired lymphatic drainage often present at these disease sites. Regardless of the mechanism, by localizing a drug to a particular site, a higher drug efficacy and lower toxicity may be realized.

Nevertheless, only a small sub-set of known drugs can be incorporated into many known drug delivery vehicles. In the case of LNPs possessing a bilayer, loading is mostly limited to multi-step, active loading techniques, and usually requires that the drugs possess amine groups. According to one active loading technique, a transmembrane pH-gradient is established such that the interior of the LNP is acidic, whereas the exterior pH-value is adjusted to physiological conditions. An uncharged amine-containing drug which is incubated with these LNPs diffuses into the vesicles and becomes charged inside the LNP due to the protonation of the amine. The charged drug can no longer pass through the bilayer and is trapped inside the LNPs. However, many widely prescribed and important drugs do not have amine groups and cannot be simply encapsulated and retained in an LNP using this method. Accordingly, the therapeutic benefits of many potentially effective drugs remain largely unrealized.

One approach to make a more wide range of drugs amenable to incorporation in a drug delivery vehicle is to conjugate them with a lipid moiety. Many drug delivery vehicles comprise hydrophobic components and the lipid moiety on the conjugate can enhance the incorporation of the drug into such components. A known strategy is to conjugate the terminal C1 carboxyl end of a fatty acid with a hydroxyl or amine group of a drug. For example, fatty acids such as squalene, stearic acid, oleic acid, palmitic acid, DHA, linoleic acid, octadecanoic acid, lauric acid and α-tocopherol have been linked to certain drugs to produce drug-lipid conjugates (as reviewed in Irby et al., 2017, “Lipid-Drug Conjugate for Enhancing Drug Delivery”, Mol. Pharm. 14(5):1325-1338). A drug can also be linked to a lipid moiety via a linker group, which serves as a spacer between the drug and the lipid. Linker groups for such purposes are known in the art and described, for example, in U.S. Pat. No. 5,149,794, which is incorporated herein by reference.

The ability to control drug release from a delivery vehicle is an important factor for achieving optimal therapeutic efficacy, it is generally known that a hydrophobic compound stays with a membrane or other hydrophobic component of a delivery vehicle more than its less hydrophobic counterpart. Thus, the overall hydrophobicity of the drug-lipid conjugate can impact its ability to be released from a drug delivery vehicle after administration. In clinical applications where a drug-lipid conjugate is required to exhibit a long circulation lifetime in the blood stream to reach a disease site, such as a distal tumour, it is important that the drug remains stably associated with the delivery vehicle for the longest time possible. Other clinical applications, such as those requiring local delivery, may require faster release. However, from a practical standpoint, it is often challenging to precisely tailor the hydrophobicity of a given molecule.

The inventors have identified a simple and broadly applicable strategy to impart desired physical properties to a drug-conjugate to enable the clinical use of many potentially effective drugs. Such strategy could be applied to a variety of other molecules of interest besides drugs as well. Examples include hydrophilic polymers, genetic material, polypeptides and proteins, such as antibodies, as well as other molecules of interest.

The compositions and methods of the present disclosure seek to address this problem and/or to provide useful alternatives to what has been previously described.

### SUMMARY

Embodiments described herein provide a scaffold molecule referred to as “L”, which forms a carbon backbone of the lipid moiety of a lipid conjugate from which one or more groups can be conjugated. The carbon backbone of L has 5 to 40 or 5 to 30 carbon atoms and optionally has one or more cis or trans C═C double bonds. L is modular in the sense that it can function as a molecular scaffold from which various combinations of a hydrocarbon group (R and/or R′) and a molecule of interest (M), including without limitation, a drug moiety (D) or polymer (optionally via a linker), can be attached via respective functional groups along its carbon backbone.

In one embodiment, the inventive approach described herein enables the hydrophobicity of a molecule of interest, such as a pro-drug, to be more precisely controlled. Without being limiting, by selecting an appropriate hydrocarbon R for conjugation to scaffold L, a molecule of interest can be designed to have a desired octanol/water Log P value.

The ability to more precisely tailor the hydrophobicity of a molecule of interest, such as drug or other molecule of interest, offers several benefits. In certain non-limiting embodiments, the inventors have shown that lipid conjugates can be designed that have a hydrophobicity such that loading into a given delivery vehicle can approach 100% encapsulation. Moreover, the retention of the lipid conjugate in a delivery vehicle after administration to a patient can also be more precisely controlled. For example, it has been found that the predicted Log P values of certain lipid conjugates described herein generally correlate with their ability to be retained in a drug delivery vehicle. Thus, by tailoring Log P values of the pro-drugs, such as by selection of an appropriate R group as described herein, more precise control of drug release can be achieved.

Generally, the lipid moiety of the molecule of interest dominates the overall hydrophobicity of the conjugate. Accordingly, a broad range of molecules can be selected for incorporation into the pro-drug. This includes drugs, polymers and other molecules of interest.

Novel pharmaceutical and drug delivery compositions comprising the lipid conjugate are also described herein. The conjugate can be incorporated into a pharmaceutical composition comprising pharmaceutically acceptable salts and/or excipients or incorporated into a drug delivery vehicle that forms a component of a pharmaceutical composition. Alternatively, the conjugate can be incorporated into a consumer product, including but not limited to a food, nutritional, cosmetic or cleaning product.

As described herein, the present disclosure is also based on the finding that LNP formulations incorporating a lipid conjugate exhibit globular electron-dense areas at the membrane. In such embodiments, the lipid nanoparticle comprises a bilayer, a lipid conjugate and a hydrophobic oil phase composed of the lipid conjugate. In one embodiment, the lipid nanoparticle is a liposome. In a further embodiment, the lipid conjugate has the structure of Formula I, Ia, II or IIa set forth herein.

In certain embodiments, provided herein is a lipid-conjugate comprising a branched lipid moiety having a backbone L that is a scaffold for linkage of one or more R hydrocarbon chains thereto, the lipid moiety having the structure of Formula IId:

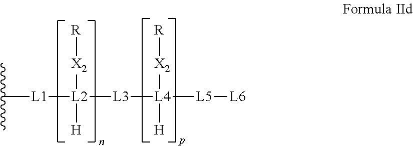

wherein the L lipid scaffold backbone is represented by L1+L2+L3+L4+L5+L6 and wherein L comprises 5 to 40 carbon atoms and 0 to 2 cis or trans C═C double bonds;

wherein L1 is a carbon chain having 3 to 30 carbon atoms and optionally L1 has one or more cis or trans C═C double bonds or 0 to 2 cis or trans C═C double bonds;

wherein L2 and L4 are each carbon atoms;

L3 is 0 to 20 carbon atoms and comprises 0 to 2 cis or trans C═C double bonds;

L5 is 0 to 20 carbon atoms and comprises 0 to 2 cis or trans C═C double bonds;

L6 is —CH₃, ═CH₂ or H;

each R is independently a linear or branched hydrocarbon chain having 0 to 30 carbon atoms and 0 to 2 cis or trans C═C double bonds, wherein if one or more of R is branched, each branch point includes an X2 functional group;

wherein n is 0 to 8 and p is 0 to 8, and wherein n+p is ≥1 or 1 to 8;

wherein each X2 is independently an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea;

or wherein X2 is a linkage that comprises at least one hydrogen bond; and

wherein the conjugate is not an ionizable lipid.

In certain embodiments, the X2 is independently a group that is biodegradable post-administration to a patient. The X2 may be independently a carbamate, ether or ester linkage.

In yet a further embodiment, L is linked to a molecule of interest M in the conjugate at LU by an X1 to form M-X1-L, wherein X1 is an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea; or wherein L is linked to the molecule of interest by a hydrogen bond between L and M of the lipid conjugate. In some embodiments, X1 is an ester, ether or carbamate.

In a further embodiment, a second L is linked to the molecule of interest by X1. Optionally, the second L has a structure of Formula IId.

In one embodiment, L1 has between 3 and 30 carbon atoms or between 4 and 30 carbon atoms.

In yet further embodiments, L is linked to the molecule of interest M by hydrogen bonds between L and M of the lipid conjugate and wherein L-X1-M has the structure of Formula V:

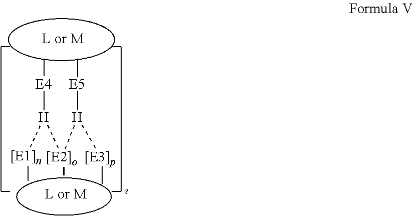

wherein E1, E2, E3, E4 and E5 are electronegative atoms independently selected from O, N and P;

E1, E2 and E3 are hydrogen bond acceptors and E4 and E5 are hydrogen bond donors;

the dotted lines depict hydrogen bonds and the solid lines depict covalent bonds;

wherein L is a lipid scaffold of the lipid moiety;

n is 0 or 1; o is 0 or 1; and p is 0 or 1; and wherein n+a+p≥2;

q is 1 to 10 or 2 to 10 or 4 to 10;

L is a lipid scaffold of the lipid moiety;

M is a molecule of interest; and

wherein E1 and E3 optionally comprise substituents linked thereto independently selected from alkyl, aryl, alkylene or H.

In one embodiment, at least one R is branched and each branch point of the R is independently selected from an ester, ether or carbamate.

In another alternative embodiment, the lipid moiety is non-cylindrical and is of a flared or frustoconical shape in a direction from L1 to L6.

According to one embodiment, X2 is not a disulfide or thioether group.

According to a further alternative embodiment, the lipid moiety is derived from a lipid having one or more reactive groups selected from a hydroxyl, amino, and/or an amide bonded to an internal carbon atom thereof to serve as the scaffold carbon chain in the lipid moiety and at least one other hydrocarbon chain in the hydrocarbon structure is derived from an acyl lipid, and wherein the X1 linkage is formed by reaction of the reactive group on the scaffold carbon chain with the carboxylic acid of the acyl chain.

Further provided herein is a lipid-conjugate comprising a branched lipid moiety having a backbone L that is a scaffold for linkage of one or more R hydrocarbon chains thereto, the lipid moiety having the structure of Formula IIe:

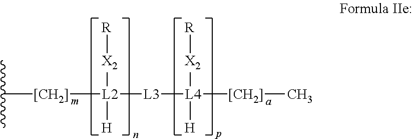

wherein L is denoted by [CH₂]ₘ-L2-L3-L4-[CH₂]_{q}— CH₃, wherein the total number of carbon atoms in L is 5 to 30;

L2 and L4 are carbon atoms;

wherein m is 0 to 20; n is 1 to 4, p is 0 to 4, and n+p is 1 to 4;

L3 is 0 to 10 carbon atoms and has 0 to 2 cis or trans C═C;

X2 are independently selected from an ether, ester and carbamate group;

wherein each R is independently:
(a) a linear or branched terminating hydrocarbon chain with 0 to 5 cis or trans C═C and 1 to 30 carbon atoms and wherein each R is conjugated to one of a respective X2 at any carbon atom in its hydrocarbon chain thereof; or
(b) a branched structure of Formula IIb having a scaffold denoted by L′:

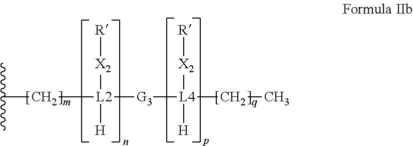

wherein L′ is denoted by [CH₂]_{f}-L2-G₃-L4-[CH₂]ᵤ—CH₃, wherein the total number of carbon atoms in L is 3 to 30;
wherein r is 0 to 20, 2 to 20, 3 to 20 or 4 to 20;
s is 0 to 4, t is 0 to 4; and wherein s+t is >1 or is 1 to 4;
u is 1 to 20;
G₃ is 0 to 10 carbon atoms and has 0 to 2 cis or trans C═C;
wherein each R′ of Formula IIb is independently a linear or branched terminating hydrocarbon chain with 0 to 5 cis or trans C═C and 1 to 30 carbon atoms;
wherein the total number of R′ hydrocarbon chains in Formula IIb is 1 to 16;

wherein each one of the R and R′ hydrocarbon chains in the lipid moiety is optionally substituted with a heteroatom, with the proviso that no more than 8 heteroatoms are substituted in the R and R′ hydrocarbon chains and wherein the predicted or experimental log P of the conjugate is greater than 5; and

wherein the lipid-conjugate is not an ionisable lipid.

According to any of the foregoing embodiments, the scaffold lipid L is derived from a hydroxy lipid.

In yet a further embodiment, the lipid conjugate has the structure of any one of the lipid conjugates depicted in FIG. 1.

According to a further aspect, there is provided a pharmaceutical composition comprising the conjugate as described above. For example, the conjugate may be formulated in a nanoparticle, such as a lipid nanoparticle. According to another embodiment, the nanoparticle comprises one or more bilayers.

Further provided is a method for treating cancer or an infection, the method comprising administering the conjugate as described above.

According to further embodiments, there is provided a pro-drug having the structure of Formula I:

M-X1-[L]-X2-R Formula I:
wherein
M is a drug moiety D;
X1 is a chemical linkage that covalently links D to any carbon atom on L;
L is a scaffold carbon chain with 5 to 40 carbon atoms and optionally having one or more cis or trans C═C double bonds;
X2 is a chemical linkage that covalently links R to any carbon atom on L; and
R is a linear or branched hydrocarbon with 1 to 40 carbon atoms and optionally having one or more, cis or trans C═C double bonds,
wherein X1 and X2 are independently selected from a functional group or a linker.

According to further embodiments, there is provided the pro-drug as described above having the structure of Formula Ia:

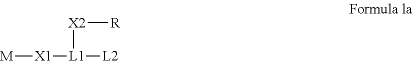

wherein
L1-L2 is the scaffold carbon chain L that has 5 to 40 carbon atoms;
L is a carbon chain having 5 to 40 carbon atoms and optionally having one or more, cis or trans C═C double bonds;
L2 is a carbon chain having L minus L1 carbon atoms and optionally having one or more, cis or trans C═C double bonds; and X2 covalently links R to L2 at any carbon atom on L2.

In some embodiments, the pro-drug has a log P of at least 5.

In alternative embodiments, the pro-drug further comprises second side R hydrocarbon chain having 1 to 40 carbon atoms covalently bonded to L via a chemical linkage X2.

The pro-drug may further comprise a third side chain R having to 1 to 40 carbon atoms covalently bonded to L via an X2 chemical linkage.

The pro-drug may comprise an R′ side chain that is linked to the first R via an X2 linkage. The pro-drug may comprise a further R′ side chain linked to another R via an X2 linkage.

The X1 and X2 linkages may be independently selected from linkages comprising one or more functional groups selected from an ester, amide, amidine, hydrazone, disulfide, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea.

The X1 and X2 linkages of the pro-drug may comprise at least one group that is biodegradable post-administration to a patient.

The pro-drug X1 in one embodiment is a linker and optionally is biodegradable.

The (M-X1) portion of Formula I or Ia may have Formula IV below:

M-[X4-M₁-X5]_{X1} Formula IV:

wherein X4 and X5 are independently selected from an ester, amide, amidine, hydrazone, disulfide, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea; and M₁ is an optional spacer group linked to the X4 and X5 functional groups and has 0 to 12 carbon atoms; or M₁ is optionally CH₂, CH₂CH₂, N-alkyl, N-acyl, O or S.

R in some embodiments is —CMe₃, -Me, or a linear carbon chain having 2 to 40 carbon atoms and optionally having 1 to 6 cis or trans double bonds.

The drug moiety D may be derived from an anti-cancer agent or an immunomodulatory agent.

The drug moiety D may be derived from docetaxel, dexamethasone, methotrexate, NPC1i, abiraterone, prednisone, prednisolone, ruxolitinib, tofacitinib, calcitriol, calcifediol, cholecalciferol, sirolimus, tacrolimus, acetylsalicylic acid, mycophenolate, cabazitaxel, betamethasone, and NLRP3 inhibitors, including CY09 (4-[[4-Oxo-2-thioxo-3-[[3-(trifluoromethyl)phenyl]methyl]-5-thiazolidinylidene]methyl]benzoic acid), INT-MA014 or MCC950 (N-(1,2,3,5,6,7-Hexahydro-s-indacen-4-ylcarbamoyl)-4-(2-hydroxy-2-propanyl)-2-furansulfonamide) or derivatives thereof, or cannabinoids, including cannabigerol, cannabichromene, cannabidiol, cannabidivarin, cannabicyclol, cannabicitran, cannabielsoin, cannabinol, tetrahydrocannabinol or tetrahydrocannabivarin or derivatives thereof.

The pro-drug may be INT-D034, INT-D035, INT-D045, INT-D046, INT-D047, INT-D048, INT-D049, INT-D050, INT-D051, INT-D050, INT-D051, INT-DOSS, INT-D056, INT-D057, INT-D058, INT-D059, INT-D060, INT-D061, INT-D062, INT-D063, INT-D064, INT-D065, INT-D066, INT-D067, INT-D053, INT-D068, INT-0069, INT-D070, INT-D071, INT-D072, INT-D073, INT-D074, INT-D075, INT-D076, INT-D077, INT-D078, INT-D079, INT-D080, INT-D081 or INT-D082.

Further provided is a pro-drug having the structure of Formula I:

M-X1-[L]-X2-R Formula I:
wherein
M is a drug moiety D derived from an anti-cancer agent or an immunomodulatory agent;
X1 is a linker comprising one or more biodegradable groups that covalently links D to any carbon atom on L;
L is a scaffold carbon chain derived from a hydroxy-fatty acid having 16 to 20 carbon atoms and optionally having one or more cis or trans C═C double bonds;
X2 is a chemical linkage that covalently links S to any carbon atom on L; and
R is a linear or branched hydrocarbon with 1 to 25 carbon atoms and optionally having one or more, cis or trans C═C double bonds, and is derived from an acyl chain,
wherein R imparts a pre-determined Log P value to the pro-drug.

Further provided is a precursor molecule P for use in the preparation of a prodrug, the precursor scaffold molecule P having the formula:

RG-[L]-X2-R Formula III:
RG is a reactive functional group comprising at least one reactive atom selected from O, C, N, P, S, Si or B;
L is a scaffold carbon chain with 5 to 40 carbon atoms and optionally has one or more cis or trans C═C double bonds;
X2 is a chemical linkage that covalently links R to any carbon atom on L; and
R is a hydrocarbon with 1 to 40 carbon atoms, and optionally has one or more, cis or trans C═C double bonds.

Further provided is a precursor molecule P as described having the formula.

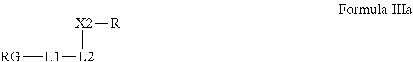

wherein
L1-L2 is the scaffold carbon chain L that has 5 to 40 carbon atoms;
L is a carbon chain having 5 to 30 carbon atoms and optionally having one or more, cis or trans C═C double bonds;
L2 is a carbon chain having L minus L1 carbon atoms and optionally having one or more, cis or trans C═C double bonds; and
X2 covalently links R to L2 at any carbon atom on L2.

RG may be a hydroxyl group, amine or carboxyl group. In one embodiment, X2 is an ester group. R may be derived from an acyl chain. In one embodiment, the first and second linkages thereby formed are ester linkages.

According to any of the foregoing embodiments, the scaffold lipid is derived from a hydroxy lipid.

Further provided is a method for preparing a pro-drug comprising: providing a precursor molecule as defined in any of the foregoing embodiments; and conjugating the precursor molecule to a drug D, a linker or a drug-linker to produce the pro-drug.

Further provided is a prodrug produced from the precursor molecule described above.

Further provided is a method for treating cancer, an autoimmune disease or infection, the method comprising administering a pro-drug of any one of the embodiments described above.

Further provided is a pharmaceutical composition comprising the lipid conjugate of any one of the embodiments described above. In additional embodiments, there is provided a nanoparticle comprising the pro-drug of an one of the embodiments described above. In an alternative embodiment, the nanoparticle is a liposome.

### BRIEF DESCRIPTION OF FIGURES

FIG. 1 depicts various pro-drugs that can be prepared according to certain embodiments based on scaffold molecule L;

FIG. 2 depicts various pro-drugs that can be prepared according to certain embodiments based on a ricinoleyl lipid scaffold;

FIG. 3 depicts chemical structures of various pro-drugs comprising a ricinoleyl lipid scaffold;

FIG. 4 shows electron microscopy images of a pro-drug comprising dexamethasone conjugated to ricinoleyl+hexanoyl (INT-D034) at various mole percentages (10, 20, 30, 40 and 80 mol %) in a lipid nanoparticle (LNP) formulation;

FIG. 5 shows electron microscopy images of a pro-drug comprising dexamethasone conjugated to ricinoleyl+hexanoyl (INT-D034; left panel) and dexamethasone conjugated to ricinoleyl+oleoyl (INT-D035; right panel) in an LNP formulation at a pro-drug concentration of 10 mol %.

FIG. 6A shows the dissociation of various ricinoleyl-dexamethasone pro-drugs formulated at 10 mol % in LNPs (INT-D034, INT-D035, INT-0045, INT-D046, INT-D047, INT-D048, INT-D049, INT-D050, INT-D051, INT-D085, INT-D086 and INT-D089) after incubation in human plasma over time. The residual amount of pro-drug in each LNP formulation was measured at 0 hr (left bar) and 2 hours (right bar) after incubation.

FIG. 68 shows the dissociation of various ricinoleyl-dexamethasone (INT-D034, INT-D045) or ricinoleyl-calcitriol (INT-D053, INT-D083) pro-drugs formulated at 10-99 mol % in LNPs after incubation in human plasma over time. The residual amount of pro-drug in each LNP formulation was measured at 0 hr (left bar) and 2 hours (right bar) after incubation.

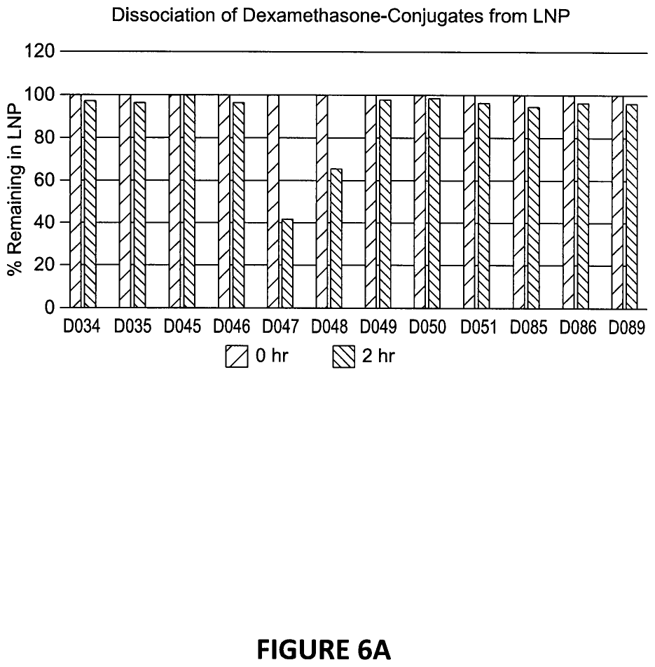

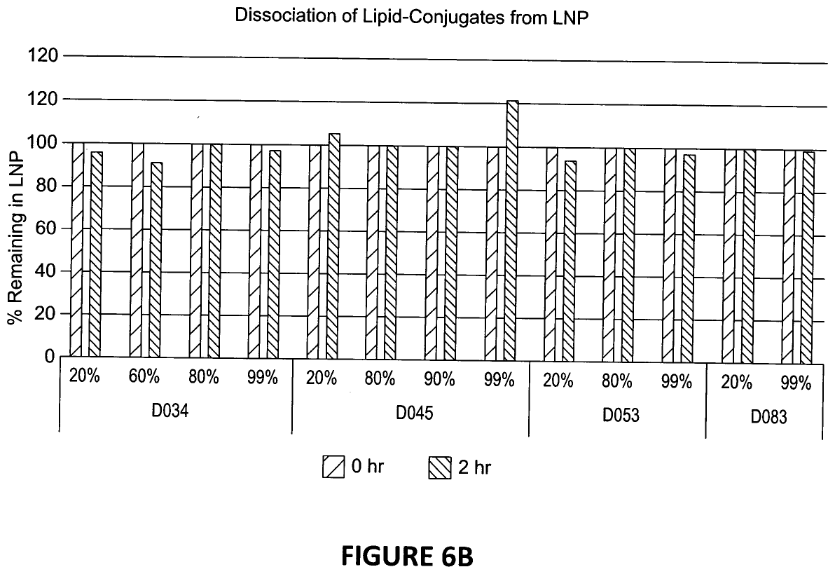

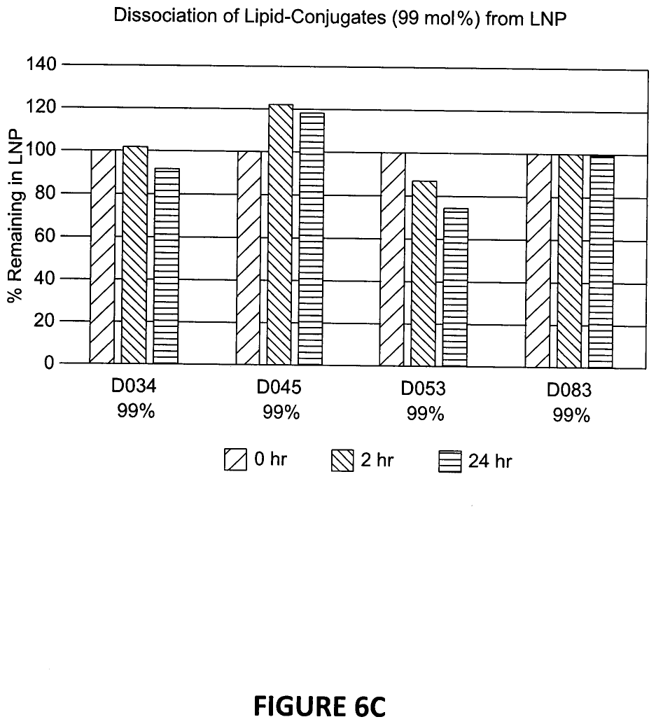

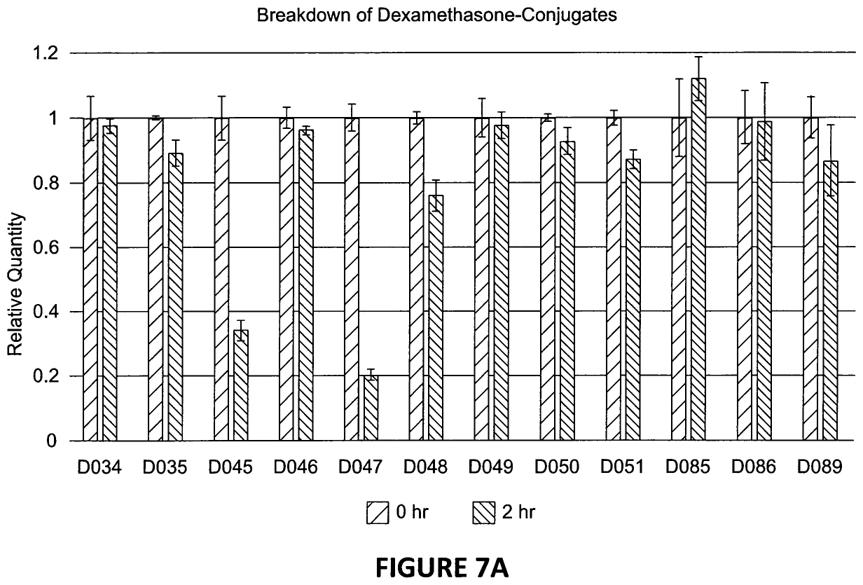

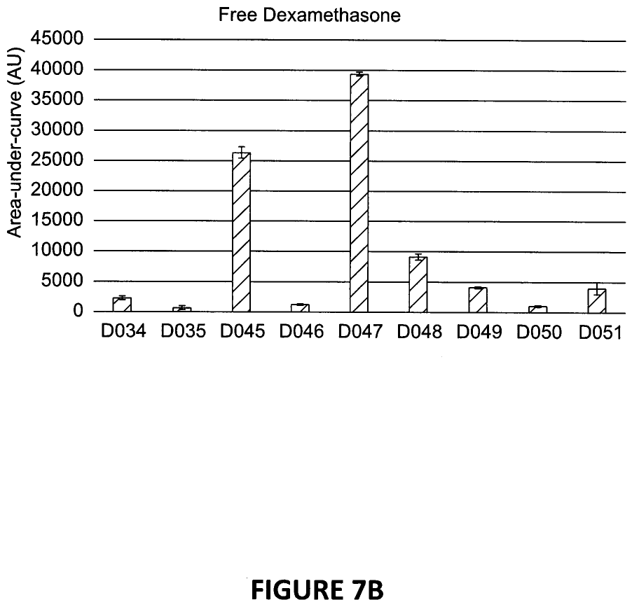

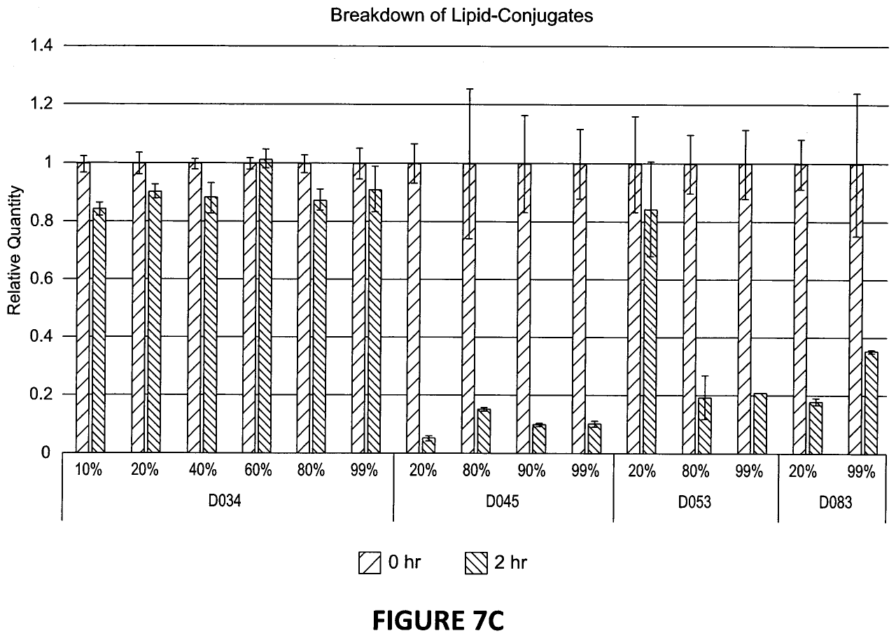

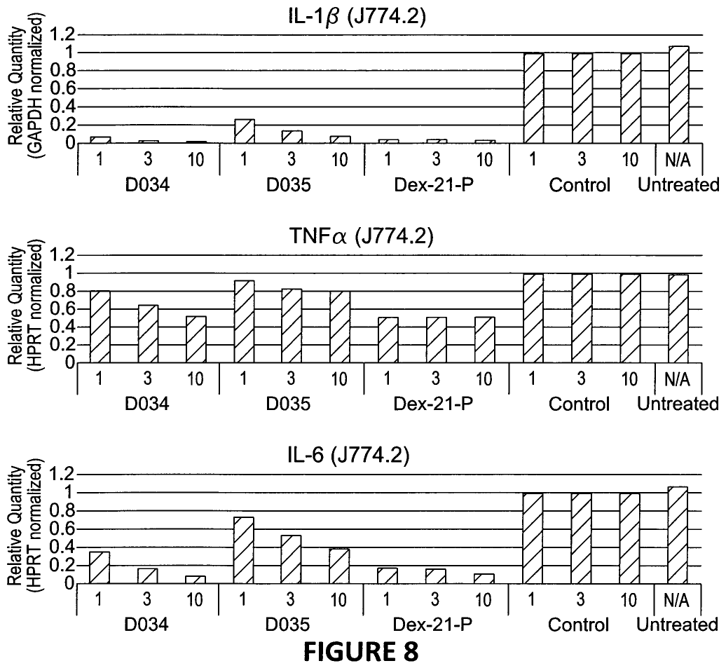

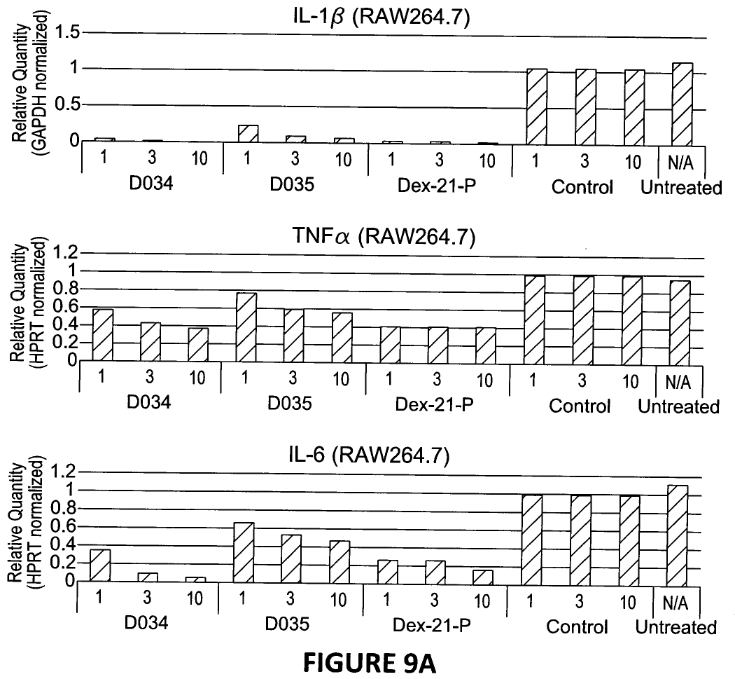

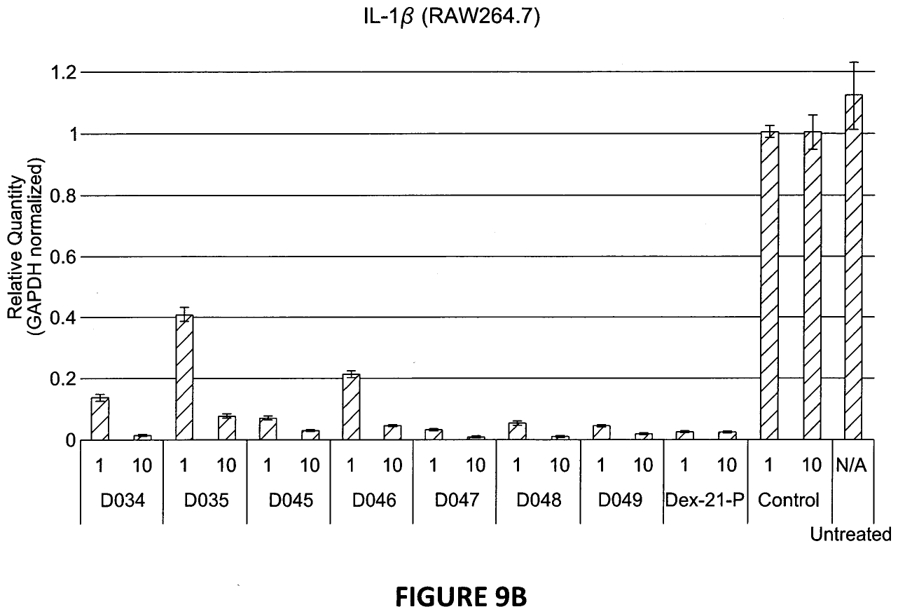

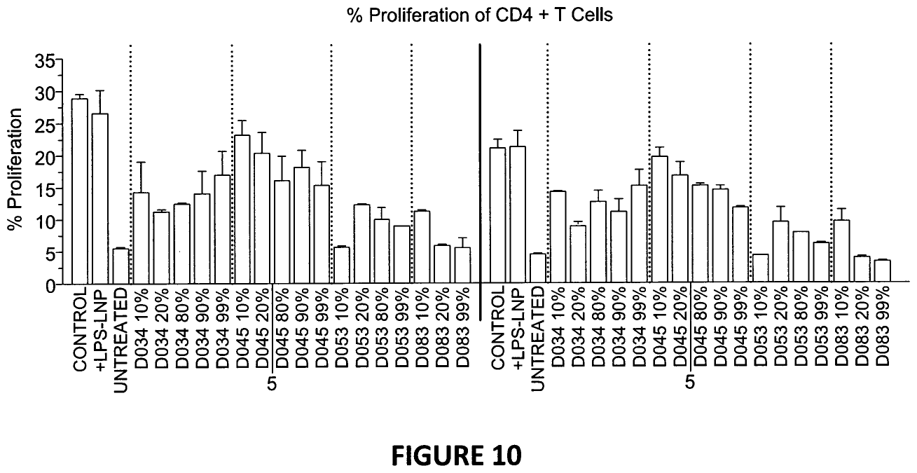

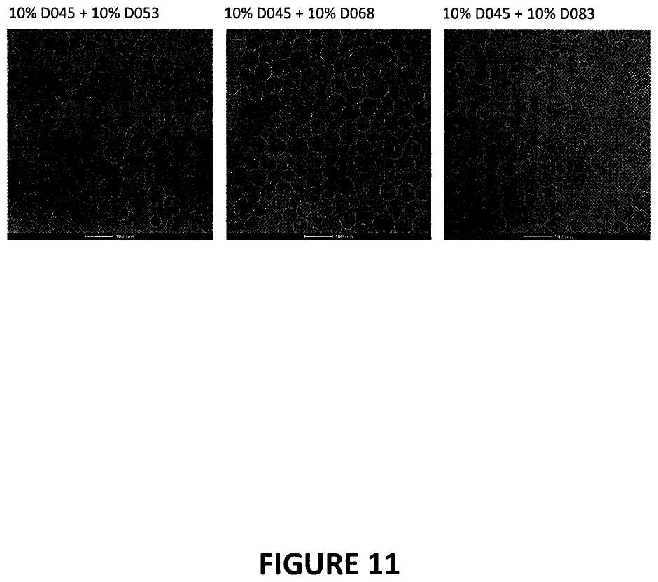

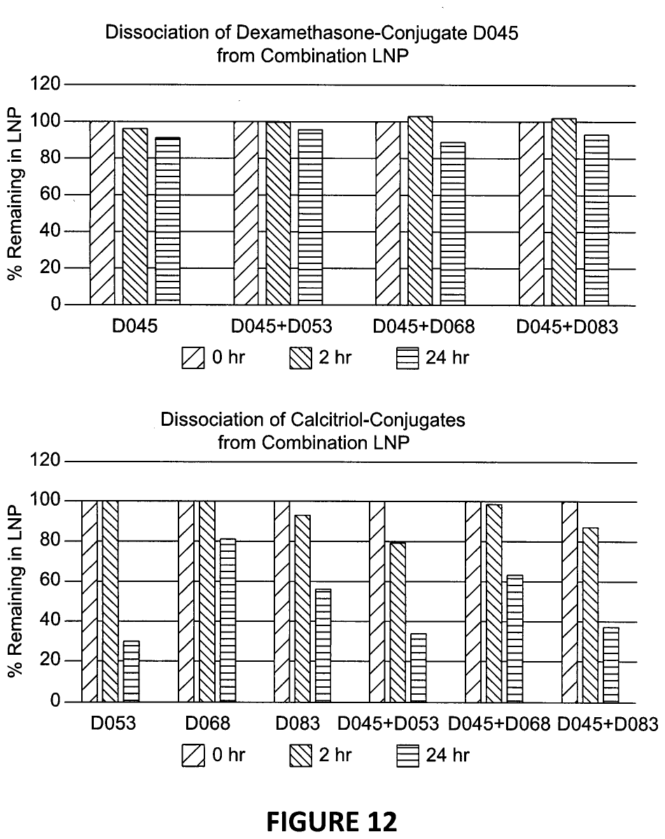

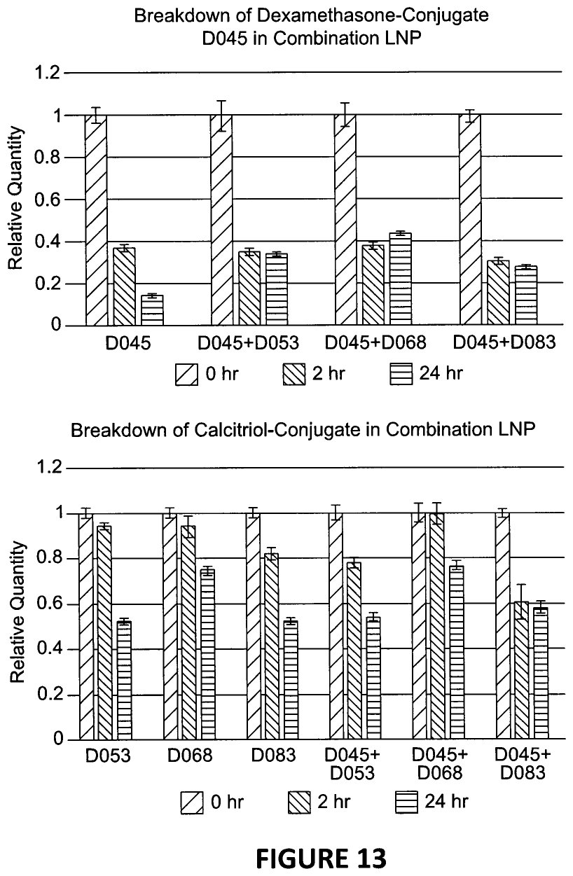

FIG. 6C shows the dissociation of various ricinoleyl-dexamethasone (INT-D034, INT-D045) or ricinoleyl-calcitriol (INT-D053, INT-0083) pro-drugs formulated at 99 mol % in LNPs after incubation in human plasma over time. The residual amount of pro-drug in each LNP formulation was measured at 0 hr (left bar), 2 hours (middle bar) and 24 hours (right) after incubation.

FIG. 7A is a graph depicting the breakdown of various ricinoleyl-dexamethasone pro-drugs formulated at 10 mol % in LNPs (INT-D034, INT-D035, INT-D045, INT-D046, INT-D047, INT-D048, INT-D049, D050, D050, D051, D085 and 0089) after incubation in mouse plasma overtime. The relative quantity of intact pro-drug in each LNP was measured at 0 hrs (left bar) and after 2 hrs (right bar) after incubation as measured by ultra high pressure liquid chromatography (UPLC). Data was normalized to the amount of the respective conjugate in the pre-incubation mixture. Error bars represent three separate sets of experiments.

FIG. 7B is a graph depicting the amount of free dexamethasone liberated after the incubation of various LNP formulated ricinoleyl-dexamethasone pro-drugs (INT-0034, INT-D035, INT-D045, INT-D046, INT-D047, INT-D048, INT-D049, INT-D050 and INT-D051) in mouse plasma overtime. The pro-drugs were formulated at 10 mol % in LNPs and the free drug was measured after 2 hours of incubation and reported as area-under-curve (AU). Error bars represent three separate sets of experiments.

FIG. 7C is a graph depicting the breakdown of various ricinoleyl-dexamethasone (INT-D034, INT-D045) or ricinoleyl-calcitriol (INT-D053, INT-D083) pro-drugs formulated 10-99 mol % in LNPs after incubation in mouse plasma over time. The relative quantity of intact pro-drug in each LNP was measured at 0 hrs (left bar) and after 2 hrs (right bar) after incubation as measured by ultra high pressure liquid chromatography (UPLC). Data was normalized to the amount of the respective conjugate in the pre-incubation mixture. Error bars represent three separate sets of experiments.

FIG. 8 shows pro-inflammatory cytokine levels of cultured macrophage cell lines J774.2 incubated with LNP formulations of the pro-drugs INT-D034 and INT-D035 (D034 and D035), free dexamethasone (Dex-21-P), LNP with no pro-drug (control) and untreated. The graph depicts the expression of the cytokines IL-1β (top), TNFα (middle) and IL-6 (bottom) after 24 hours of incubation of the cells with the above components at doses equivalent to 1, 3 or 10 μM of dexamethasone, followed by stimulation by 10 ng/mL lipopolysaccharide (LPS) overnight. Cytokine levels were measured by qRT-PCR and data was normalized to cells treated with control LNP without drug-lipid conjugates.

FIG. 9A shows pro-inflammatory cytokine levels of Raw264.7 cells incubated with LNP formulations of the pro-drugs INT-D034 and INT-D035 (D034 and D035), free dexamethasone (Dex-21-P), LNP with no pro-drug (control) and untreated. The graph depicts the expression of the cytokines IL-10 (top), TNFα (middle) and IL-6 (bottom) after 24 hours of incubation of the cells with the above components at doses equivalent to 1, 3 or 10 μM of dexamethasone, followed by stimulation by 10 ng/mL LPS overnight. Cytokine levels were measured by qRT-PCR and data was normalized to cells treated with control LNP without drug-lipid conjugates.

FIG. 9B shows pro-inflammatory cytokine levels of Raw264.7 cells incubated with LNP formulations of the pro-drugs INT-D034, INT-D035, INT-D045, INT-D046, INT-0047, INT-D048 and INT-D049 (D034, D045, D046, D047, D048 and D049), free dexamethasone (Dex-21-P), LNP with no pro-drug (control) and untreated. The graph depicts the expression of the cytokines IL-10 after 24 hours of incubation of the cells with the above components at doses equivalent to 1 or 10 μM of dexamethasone, followed by stimulation by 10 ng/mL LPS overnight. Cytokine levels were measured by qRT-PCR and data was normalized to cells treated with control LNP without drug-lipid conjugates.

FIG. 10 shows the percentage proliferation of CD4+ T cells for various LNP formulations of the pro-drugs of dexamethasone (INT-D034 and INT-D045) and calcitriol (INT-D053 and INT-D083) at various mol % from 10 to 99% as indicated in a mixed lymphocyte (MLR) reaction assay. Bone marrow derived dendritic cells (BMDCs) from C57Bl/6 male mice were first treated with LNP containing various mol % of the dexamethasone or calcitriol conjugates for 48 hours and then activated by incubation with LIPS for 24 hours. They were then harvested and mixed with CD4+ T cells isolated from Balb/cJ male mice (Jackson Laboratories) at 5:1 or 10:1 T-to-BMDC ratio.

FIG. 11 is electron microscopy images of LNPs loaded with two different pro-drugs derived from different parent drug moieties, namely dexamethasone and calcitriol. The pro-drugs included: INT-D045 and INT-D053 (left panel); INT-D045 and INT-D068 (middle panel); and INT-D045 and INT-083 (right panel). Each pro-drug was formulated in an LNP at equimolar concentrations of 10 mol %.

FIG. 12 shows the dissociation of ricinoleyl-dexamethasone (INT-D045) or ricinoleyl-calcitriol (INT-D053, INT-D068 or INT-D063) conjugates formulated at 10 mol % individually or in combination in LNPs. The residual amount of lipid-drug conjugate in each LNP formulation was measured at 0 hr (left bar), 2 hours (middle bar) and 24 hours (right) after incubation in human plasma over time. Top graph indicates levels of dexamethasone conjugate in single or combination formulations. Bottom graph indicates levels of calcitriol conjugate in single or combination formulations. Data was normalized to the amount of the respective conjugate in the pre-incubation mixture.

FIG. 13 is a graph depicting the breakdown of ricinoleyl-dexamethasone (INT-0045) or ricinoleyl-calcitriol (INT-D053, INT-D068 or INT-D083) conjugates formulated at 10 mol % individually or in combination in LNPs. The relative quantity of intact pro-drug in each LNP was measured by UPLC at 0 hrs (left bar), 2 hours (middle bar) and 24 hours (right bar) after incubation in mouse plasma. Top graph indicates levels of dexamethasone conjugate in single or combination formulations. Bottom graph indicates levels of calcitriol conjugate in single or combination formulations. Data was normalized to the amount of the respective conjugate in the pre-incubation mixture. Error bars represent three separate sets of experiments.

### DETAILED DESCRIPTION

### Lipid Conjugates of Formula I

The lipid conjugate described herein can be a pro-drug, which in certain embodiments refers to a compound that can become active after administration to a subject. However, other molecules of interest M besides a drug moiety can be conjugated to the lipid moiety, such as a polymer as described herein. Regardless of the molecule of interest, the lipid conjugate comprises a scaffold L, which is a carbon chain that is typically linear, although branched structures are encompassed by the compositions described herein as well. The molecule of interest M is linked to L via chemical linkage X1, which may include direct linkage or a linker in some embodiments. An R hydrocarbon is linked to L via chemical linkage X2. Optionally a second R hydrocarbon is linked to L via an X2 chemical linkage. Yet further, a third R hydrocarbon is optionally linked to L via a chemical linkage as described below.

In one embodiment, the lipid conjugate has the structure of Formula I set forth below.

M-X1-[L]-X2-R Formula I:

wherein

M is a molecule of interest, including a drug or polymer;

X1 is any chemical linkage or linkages that links M to any carbon atom on L, including a bond that is covalent or ionic, or that comprises a hydrogen bond or bonds;

L is a scaffold carbon chain with 5 to 40 carbon atoms and optionally has one or more cis or trans C═C double bonds;

X2 is a chemical linkage that covalently links R to any carbon atom on L; and

R is a hydrocarbon with 1 to 40 carbon atoms, and optionally having one or more, cis or trans C═C double bonds, and

optionally a second R hydrocarbon with 1 to 40 carbon atoms, and optionally having one or more, cis or trans C═C double bonds is chemically linked to L via an X2 chemical linkage. Yet further, optionally, a third R hydrocarbon with 1 to 40 carbon atoms, and optionally having one or more, cis or trans C═C double bonds is chemically linked to L via an X2 chemical linkage.

Optionally a side chain R′ is linked to any one of the hydrocarbons R via an X2 chemical linkage. Without being limiting, a second R′ side chain may be linked to an R hydrocarbon via an X2 linkage and a third R′ may be linked to any one of the hydrocarbons R via an X2 chemical linkage. Various other combinations could be readily envisioned by those of skill in the art. Chemical linkages X2 may include any suitable functional group and/or a linker as described below, as well as others known to those of skill in the art.

In a further embodiment R, and/or the optional additional R or R′ groups, independently are hydrocarbon chains that have 1 to 40 carbon atoms, 2 to 30 carbon atoms or 5 to 25 carbon atoms. Likewise, the L scaffold (described below) may have 1 to 40 carbon atoms, 2 to 30 carbon atoms or 5 to 25 carbon atoms.

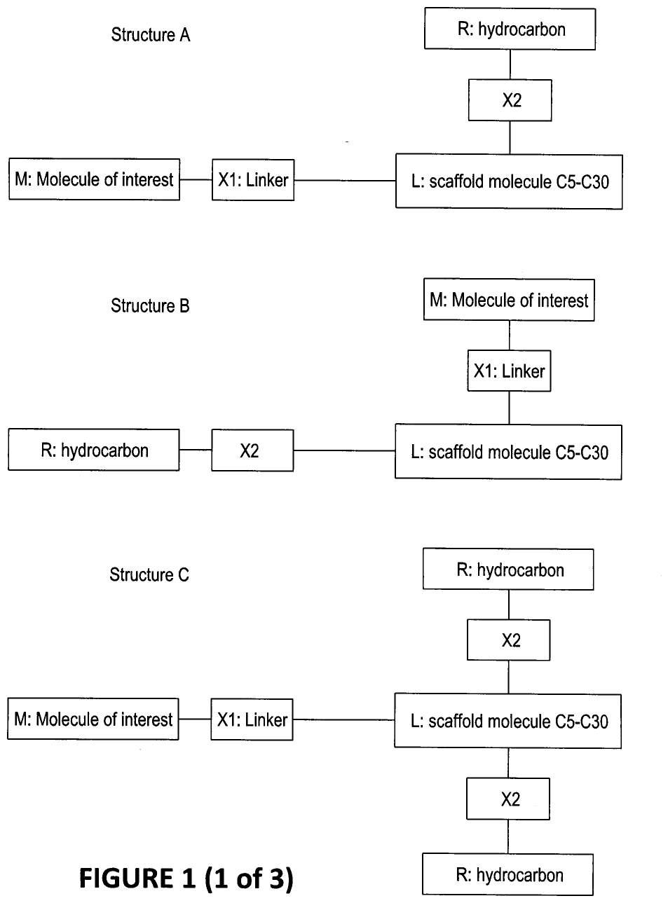

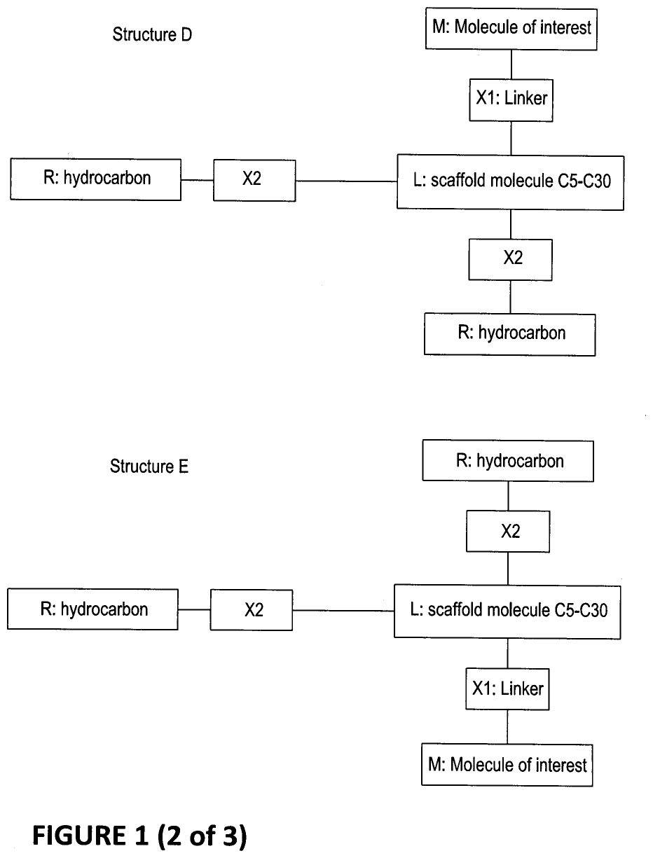

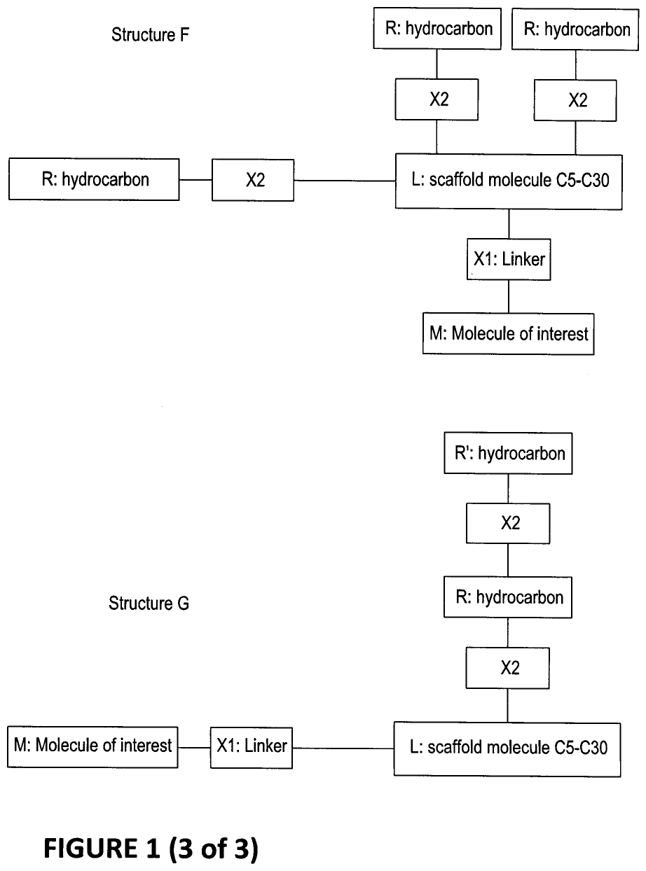

The diagrams in FIG. 1 are presented to pictorially demonstrate a variety of different lipid conjugates of Formula I, Ia, II and IIa that can be created in select embodiments using the inventive approach described herein. As shown, a molecule of interest M or a molecule-linker, R hydrocarbon and an optional second R hydrocarbon, or an optional further third R hydrocarbon, can occupy various positions on the scaffold backbone L to provide a tailored pro-drug. As further described (and noted above), one or more of the hydrocarbons R linked to the scaffold L may have further carbon-based side chains attached thereto.

Although the structures depicted in FIG. 1 utilize a linker X1 (also referred to in the art as a “spacer”) to chemically link the molecule of interest to the scaffold molecule L, optionally the molecule of interest M can be directly linked to L via an X1 functional group. In addition, the chemical linkage X1 may include any combination of a linker and one or more functional groups as described further below.

In particular, Structure A in FIG. 1 shows a scaffold molecule L, which in this non-limiting example has 5 to 30 carbon atoms, in which a terminal carbon atom is linked to molecule of interest M via an X1 chemical linkage that is a linker. A hydrocarbon R is linked to an internal carbon of the scaffold carbon chain L via an X2 linkage.

Structure B of FIG. 1 depicts a scaffold molecule L having 5 to 30 carbon atoms in which a terminal carbon atom is linked to the hydrocarbon R via the X2 chemical linkage (rather than the molecule of interest M and linker). The molecule of interest M is linked to an internal carbon of the scaffold via an X1 chemical linkage that is a linker.

Similar to Structure A, the structure depicted in Structure C of FIG. 1 shows a scaffold molecule L in which a terminal carbon atom is linked to a molecule of interest M via a linker X1 and in which the hydrocarbon R is linked to an internal carbon of the scaffold via an X2 linkage. However, in this embodiment, a second hydrocarbon R is linked to another internal carbon of the scaffold via an X2 linkage.

In structure D, a scaffold molecule L is depicted in which the molecule of interest M is linked to an internal carbon of the scaffold via an X1 chemical linkage that is a linker. The hydrocarbon R is linked to an internal carbon of the scaffold via an X2 linkage. A second hydrocarbon R′ is linked to a terminal carbon atom of the scaffold L via an X3 chemical linkage.

Structure E of FIG. 1 depicts a scaffold molecule L in which the molecule of interest M is linked to an internal carbon of the scaffold via an X1 chemical linkage that is a linker. The hydrocarbon R is linked to an internal carbon of the scaffold via an X2 linkage. A second hydrocarbon R is linked to a terminal carbon atom of the scaffold L via an X2 chemical linkage. Structure E differs from Structure D above in that the molecule of interest M is linked to a carbon atom on scaffold L at a position that is closer to the terminal carbon than the position at which second R hydrocarbon is linked.

In another example, Structure F of FIG. 1 depicts a scaffold molecule L in which the molecule of interest M is linked to an internal carbon of the scaffold via an X1 chemical linkage that is a linker. The hydrocarbon R is linked to an internal carbon of the scaffold via an X2 linkage. A second hydrocarbon R is linked to a terminal carbon atom of the scaffold L via an X2 chemical linkage. Structure F differs from Structure E above in that a third hydrocarbon R is linked to scaffold L via an X2 chemical linkage. It will be readily envisioned that other combinations could include a drug-linker at C1 and three hydrocarbon moieties linked to internal carbons of L via respective X2 chemical linkages.

In yet a further example shown in Structure G of FIG. 1, an R hydrocarbon has linked thereto an R′ hydrocarbon side chain linked via X2. A terminal carbon atom is linked to molecule of interest M via an X1 chemical linkage that is a linker. The hydrocarbon R is linked to an internal carbon of the scaffold carbon chain L via an X2 linkage.

The above structures A to G are examples in that other permutations and embodiments falling within the scope of the disclosure can be readily envisioned by those of skill in the art.

The point on scaffold L at which group R is linked may in some embodiments be at least 3 carbon atoms from a terminal carbon on L (as measured from a first carbon of L referred to as C1). To depict such a branch-point in the chemical formula of the pro-drug (Formula I above), the scaffold molecule L may be referred to using the notation “L1-L2”. According to such embodiment, L1 is at least 3 carbon atoms and S is linked to a carbon atom of L2. In one particularly advantageous embodiment, L1 is at least 4 or S carbon atoms.

In those embodiments in which the group R is linked to L at a position that is at least 3 carbon atoms from C1, Formula I may take the form of Formula Ia below:

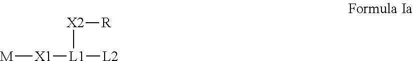

wherein M is a molecule of interest; X1 is a chemical linkage that conjugates or links M to any carbon atom on L1-L2 via any appropriate chemical linkage described herein; L2 is at least 3 carbon atoms; L1-L2 is 5 to 40 carbon atoms; and L2=L−L1. The chemical linkage X1 conjugates the molecule of interest M to any carbon atom on L1-L2 and the chemical linkage X2 conjugates R to any carbon atom on L2. R is a hydrocarbon having 1 to 40 carbon atoms.

In one embodiment, L1 is 3, 4, 5, 6, 7, 8, 9, 10, 11, 12, 13, 14, 15, 16, 17, 18, 19, 20, 21, 22, 23, 24, 25, 26, 27, 28, 29 or 30 carbon atoms, in further embodiments, L1 may be 3 to 30 carbon atoms, 4 to 30 carbon atoms, 5 to 25 carbon atoms, or 6 to 25 carbon atoms, or 7 to 20 carbon atoms. Optionally L1 has one or more cis or trans C═C double bonds. In another embodiment, L1 is a linear carbon chain.

While L2 is typically a linear carbon chain, branched structures are contemplated as well. As discussed above, L2=L−L1. To illustrate, in those embodiments in which L is 20 carbon atoms and L1 is 11 carbon atoms, L2 is 9 carbon atoms.

In an alternative embodiment, the lipid conjugate has a lipid moiety of the structure of Formula II as set forth below.

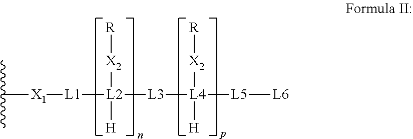

wherein the L lipid scaffold backbone is represented by L1+L2+L3+L4+L5+L6 and wherein L comprises 5 to 40 carbon atoms or 5 to 30 carbon atoms or 5 to 25 carbon atoms or 5 to 20 carbon atoms and 0 to 2 cis or trans C═C double bonds;

wherein L1 is a carbon chain having 1 to 30 carbon atoms, 3 to 30 carbon atoms, 4 to 30 carbon atoms, 5 to 25 carbon atoms, 6 to 25 carbon atoms or 7 to 20 carbon atoms, and optionally L1 has one or more cis or trans C═C double bonds or 0 to 2 cis or trans C═C double bonds;

wherein L2 and L4 are each carbon atoms;

L3 is 0 to 20 carbon atoms and comprises 0 to 2 cis or trans C═C double bonds;

L5 is 0 to 20 carbon atoms and comprises 0 to 2 cis or trans C═C double bonds;

L6 is —CH₃, —CH₂ or H;

each R is independently a linear or branched hydrocarbon chain having 0 to 30 carbon atoms and 0 to 2 cis or trans C═C double bonds, wherein according to one alternative embodiment, each R is independently branched with each branch point including an X2 functional group comprising a heteroatom;

wherein n is 0 to 8 and p is 0 to 8, and wherein n+p is ≥1 or 1 to 8 or wherein n is 0 to 6 and p is 0 to 6, and wherein n+p is ≥1 or 1 to 6 or wherein n is 0 to 4 and p is 0 to 4 and wherein n+p is ≥1 or 1 to 4; and

X1 and X2 are independently selected from an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea; or wherein X1 comprises one or more hydrogen bonds and has the structure of Formula V defined below.

In one embodiment, at least one of X1 and X2 is biodegradable.

In one embodiment, X1 and/or X2 is independently selected from an ester, ether or carbamate. The ester or carbamate may be in any orientation. For example, the ester may be linked to the molecule of interest (M) via its carbonyl group or via its 0 group. Likewise, the carbamate can be linked to the molecular of interest (M) via its nitrogen atom or via its —O— group.

In one embodiment, the lipid moiety of Formula II in total has less than 300, less than 200, less than 150, less than 100 carbon atoms, less than 75 carbon atoms, or less than 50 carbon atoms (L+R).

Each one of the R hydrocarbon chains in the lipid moiety is optionally substituted with a heteroatom at one of its internal carbon atoms in its chain, with the proviso that no more than 8 heteroatoms are substituted in the R hydrocarbon chains of the lipid moiety. In another embodiment, the predicted or experimental log P of the conjugate is greater than 5.

In yet a further embodiment, the lipid-conjugate is not an ionisable lipid.

In an alternative embodiment, the lipid conjugate has a lipid moiety of the structure of Formula IIa as set forth below.

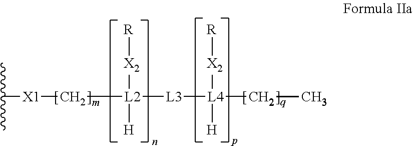

wherein L is denoted by [CH₂]ₘ-L2-L3-L4-[CH₂]_{q}— CH₃, wherein the total number of carbon atoms in L is 5 to 30;

L2 and L4 are carbon atoms;

wherein m is 0 to 20; n is 1 to 4, p is 0 to 4, and n+p is 1 to 4;

L3 is 0 to 10 carbon atoms and has 0 to 2 cis or trans C═C;

X1 and X2 are independently selected from an ether, ester and carbamate group;

wherein each R is independently:
(a) a linear or branched terminating hydrocarbon chain with 0 to 5 cis or trans C═C and 1 to 30 carbon atoms and wherein each R is conjugated to one of a respective X2 at any carbon atom in its hydrocarbon chain thereof, or
(b) a branched structure of Formula IIb having a scaffold denoted by L′;

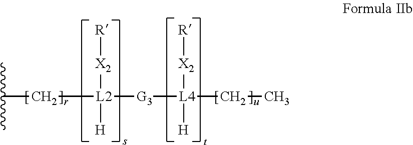

wherein L′ is denoted by [CH₂]ₜ-L2-G₃-L4-[CH₂]ᵤ—CH₃, wherein the total number of carbon atoms in L is 3 to 30; and
wherein r is 0 to 20, 2 to 20, 3 to 20 or 4 to 20;
s is 0 to 4, t is 0 to 4; and wherein s+t is >1 or is 1 to 4;
u is 1 to 20;
G₃ is 0 to 10 carbon atoms and has 0 to 2 cis or trans C═C;
wherein each R′ of Formula IIb is independently a linear or branched terminating hydrocarbon chain with 0 to 5 cis or trans C═C and 1 to 30 carbon atoms;
wherein the total number of R′ hydrocarbon chains in Formula IIb is 1 to 16;

wherein each one of the R and R′ hydrocarbon chains in the lipid moiety is optionally substituted with a heteroatom, with the proviso that no more than 8, 6, 4 or 2 heteroatoms are substituted in the R and R′ hydrocarbon chains and wherein the predicted or experimental log P of the conjugate is greater than 5; and

wherein the lipid-conjugate is not an ionisable lipid.

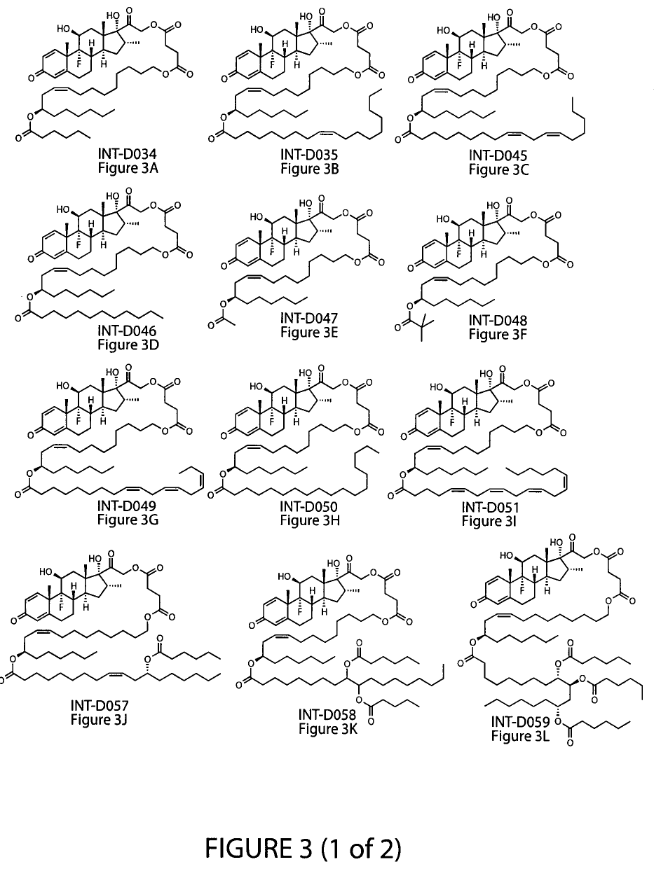

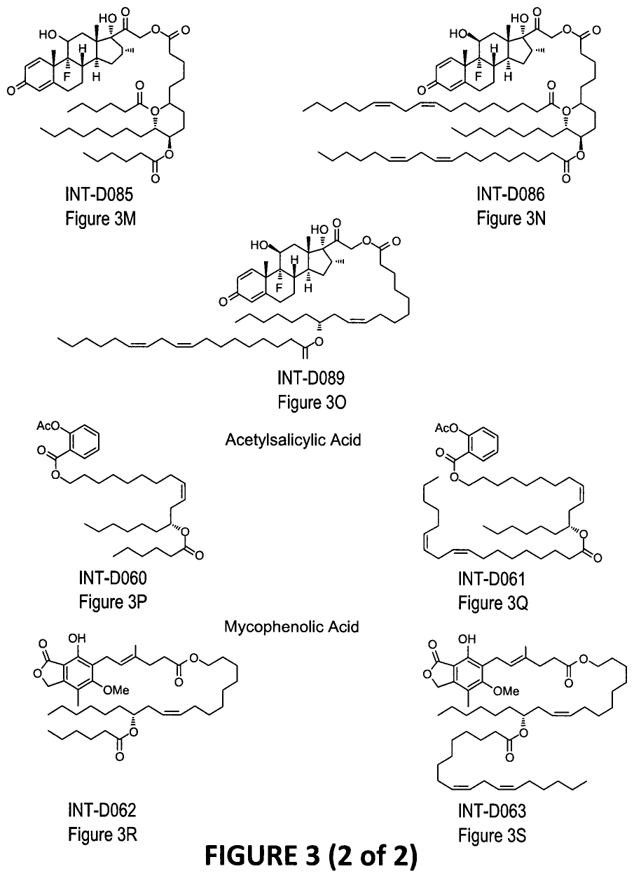

Non-limiting examples of pro-drug lipid conjugates having the structures of Formula I, Formula Ia, Formula II and Formula IIe are provided in Table 1 below, and their chemical structures are provided in FIG. 3. In such embodiments, the lipid conjugates are derived from dexamethasone and employ a succinate linker (X1 chemical linkage), although a broad range of drugs or other molecules of interest and linkers can be incorporated into the lipid conjugate as discussed further herein.

TABLE 1
Non-limiting examples of pro-drugs

| Identifier | | LogP | |
|---|---|---|---|
| of pro-drug | L and R of Formula I, Ia, II or IIa | (predicted) | Figure |
| INT-D034 | Ricinoleyl + hexanoyl (C6:0) | 10.37 | 3A |
| INT-D035 | Ricinoleyl + oleoyl (C18:1) | 15.34 | 3B |
| INT-D045 | Ricinoleyl + linoleoyI (C18:2) | 14.98 | 3C |
| INT-D046 | Ricinoleyl + lauroyl (C12:0) | 13.04 | 3D |
| INT-D047 | Ricinoleyl + acetyl (C2:0) | 8.33 | 3E |
| INT-D048 | Ricinoleyl + pivaloyl (C5:0) | 10.13 | 3F |
| INT-D049 | Ricinoleyl + linolenoyl (C18:3) | 14.62 | 3G |
| INT-D050 | Ricinoleyl + stearoyl (18:0) | 15.7 | 3H |
| INT-D051 | Ricinoleyl + arachldonoyl (C20:4) | 15.14 | 3I |
| INT-D057 | Ricinoleyl + ricinoleoyl + | 16.43 | 3J |
| | hexanoyl (C6:0) | | |
| INT-D058 | Ricinoleyl + 9,10- | 17.75 | 3K |
| | dihydroxystearoyl + hexanoyl | | |
| | (C6:0) | | |
| INT-D059 | Ricinoleyl + 9,10,12- | 19.21 | 3L |
| | trihydroxystearyl + hexanoyl | | |
| | (C6:0) | | |
| INT-D085 | 9,10-dihydroxystearoyl + | 11.98 | 3M |
| | hexanoyl (C6:0) | | |
| INT-D086 | 9,10-dihydroxystearoyl + | 21.2 | 3N |
| | linolenoyl (C18:3) | | |
| INT-D089 | Ricinoleoyl + linolenoyl (C18:3) | 14.83 | 3O |
| INT-D060 | Ricinoleyl + hexanoyl (C6:0) | 9.86 | 3P |
| INT-D061 | Ricinoleyl + linoleoyl (C18:2) | 14.47 | 3Q |
| INT-D062 | Ricinoleyl + hexanoyl (C6:0) | 11.95 | 3R |
| INT-D063 | Ricinoleyl + linoleoyl (C18:2) | 16.56 | 3S |

### Scaffold L of Formula I, Ia, II or IIa

In one embodiment, the L of Formula I, Ia, II or IIa is derived from a fatty acid with a functional group for linkage to R on its carbon chain.

For example, L of formula I, Ia, II or IIa may be derived from a hydroxy fatty acid (HFA), which is a fatty acid having an OH group bonded at any position on its carbon chain. Without being limiting, the HFA may be an α-hydroxy fatty acid, a β-hydroxy fatty, a ω-hydroxy fatty acid or any (ω-1)-hydroxy fatty acid, or any other known HFA, The HFA may be saturated or unsaturated. Two or more hydroxy functional groups can be present on the carbon chain as well.

Non-limiting examples of HFAs from which fatty alcohols can be derived are set forth in Table 2 below:

TABLE 2
Examples of hydroxy fatty acids (HFAs) and corresponding fatty alcohols

| | | Abbreviation | Corresponding fatty |
|---|---|---|---|
| Systematic name of fatty acid | Common name of fatty acid | of fatty acid | alcohol name |
| 2-Hydroxyhexadecanoic acid | α-Hydroxypalmitic acid | 2-OH-16:0 | α-Hydroxypalmitoleyl alcohol |
| 3-Hydroxyhexadecanoic acid | β-Hydroxypalmitic acid | 3-OH-16:0 | β-Hydroxypalmitoleyl alcohol |
| 3-Hydroxyoctadecanoic acid | β-Hydroxystearic acid | 3-OH-18:0 | β-Hydroxystearyl alcohol |
| 12-Hydroxyoctadecanoic acid | 12-Hydroxystearic acid | 12-OH-18:0 | 12-Hydroxystearyl alcohol |
| 17-Hydroxyoctadecanoic acid | 17-Hydroxystearic acid | 17-OH-18:0 | 1-Hydroxystearyl alcohol |
| 12-hydroxy-cis-9-octadecenoic acid | Ricinoleic acid | 12-OH-18:1 | Ricinoleyl alcohol |
| 12-hydroxy-trans-9-octadecenolc acid | Ricinelaidic acid | 12-OH-18:1 | Ricinelaidic alcohol |
| 14-hydroxy-cis-11-eicosenoic acid | Lesquerolic acid | 14-OH-20:1 | Lesquerolic alcohol |

Examples of HFAs with two or more hydroxy functional groups present in the carbon chain include 9,10-dihydroxyoctadecanoic acid and ustilic acid (also known as 2,15,16-trihydroxy palmitic acid or 2,15,16-trihydroxy-hexadecanoic acid).

The L of Formula I, Ia, II or IIa is alternatively derived from branched fatty acid esters of HFAs known in the art as fatty acid esters of hydroxyl fatty acids (FAHFAs). These fatty acids esters comprise a branched ester linkage between a fatty acid and an HFA. For example, 9-[(9Z)-octadecenoyloxy]octadecanoic acid is a fatty acid ester obtained by condensation of the carboxy group of oleic acid with the hydroxy group of 9-hydroxyoctadecanoic acid.

In alternative embodiments, L is derived from a fatty acid amide, which may comprise ethanolamine as the amine component.

The L of Formula I, Ia, II or IIa may be derived from other fatty acids besides those described above. In addition, it will be appreciated that the fatty acids, in turn, can be derived from their corresponding tri-glycerides.

The L of Formula I, Ia, II or IIIa may include OH groups that are introduced via oxidation of a double bond on a lipid carbon backbone. Thus, the precursor for L can be derived from any fatty acid, fatty alcohol or fatty amide precursor that is unsaturated and oxidized to introduce reactive OH groups.

The lipid moiety of the lipid conjugate, such as a pro-drug or lipid-polymer conjugate, may be compatible with lipids for incorporation into a drug delivery vehicle. For example, this may include compatibility with vesicle forming lipids, such as phospholipids, that form part of a lipid nanoparticle, such as a liposome. The lipid moiety may also be compatible with other drug delivery vehicles such as polymer-based nanoparticles, emulsions, micelles and nanotubes.

In one embodiment, the L may be derived from a precursor fatty acid or other molecule having, for example, 5 to 30 carbon atoms, 14 to 20 carbon atoms or 16 to 18 carbon atoms.

Lipid-Based Precursor P

In an alternative embodiment, the lipid moiety of the lipid conjugate of Formula I above may be derived from a precursor, referred to herein as “P” defined by Formula III below:

RG-[L]-X2-R Formula III:

wherein RG is a reactive functional group comprising one or more reactive atoms selected from O, C, N, P, S, Si or B. In one embodiment, the reactive functional group is selected from a hydroxyl, amine or a carboxyl group. In another embodiment, the reactive functional group is a hydroxyl or carboxyl group. In an alternative embodiment, the RG functional group forms a biodegradable chemical linkage with a linker on the molecule of interest M or directly with such molecule.

L is a scaffold carbon chain with 5 to 40 carbon atoms and optionally has one or more cis or trans C═C double bonds;

X2 is a chemical linkage that covalently links R to any carbon atom on L; and

R is a hydrocarbon with 1 to 40 carbon atoms, and optionally having one or more, cis or trans C═C double bonds.

In another embodiment, the lipid moiety of Formula Ia may be derived from precursor P having a structure of Formula IIIa:

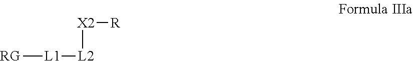

wherein RG is a reactive functional group comprising at least one reactive atom selected from O, C, N, P, S, Si or B. In one embodiment, the reactive functional group is selected from a hydroxyl, amine or a carboxyl group. In another embodiment, the reactive functional group is a hydroxyl, or carboxyl group.

In an alternative embodiment, the RG functional group forms a biodegradable chemical linkage with a linker on a drug or with a drug. In a further alternative embodiment, the RG functional group is a hydrogen bond donor or acceptor group. L1 is at least 3 carbon atoms; L1-12 is 5 to 40 carbon atoms; and L2=L−L1. The chemical linkage X2 conjugates R to any carbon atom on L2, R is a hydrocarbon having 1 to 40 carbon atoms.

In one non-limiting embodiment, RG in Formula III or IIIa is a hydroxyl group. RG may become conjugated with a corresponding reactive group on a drug or a linker, such as a carboxyl group. The bond formed (X1 of Formula I or Ia) upon such reaction may be selected from an ester or amide bond, although other bonds could be formed as well.

The carbon backbone of L in Formula III or L1-L2 in Formula IIIa may also include a further reactive group RG for linkage of a second hydrocarbon R group. Moreover, a third hydrocarbon group R may be linked to the carbon backbone of L via an RG. Likewise, the second or third reactive groups RG may comprise one or more atoms selected from O, C, N, P, S, SI or B. In one non-limiting example, each RG is independently selected from a hydroxyl, amine, or carboxylic acid group, as well as other suitable groups known to those of skill in the art.

Moreover, two or more of the hydrocarbon moieties, R of Formula III and IIIa may have linked thereto a respective R′ side chain. For example, an R′ side chain may be linked to an R via an X2 linkage and a second R′ side chain may be linked to another R via an X2 linkage and/or a third R′ may be linked to any R via an X2 as described previously in connection with Formulas I, Ia, II and IIa. However, various other combinations could be readily envisioned by those of skill in the art.

In another embodiment, the lipid moiety of Formula II may be derived from precursor P having a structure of Formula IIIb:

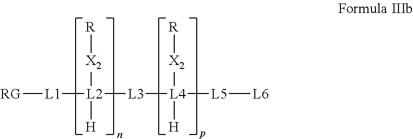

wherein RG is a reactive functional group comprising one or more reactive atoms selected from O, C, N, P, SI or 5. In one embodiment, the reactive functional group is selected from a hydroxyl, amine or a carboxyl group. In another embodiment, the reactive functional group is a hydroxyl or carboxyl group. In an alternative embodiment, the RG functional group forms a biodegradable chemical linkage with a linker on a drug or with a drug. In a further embodiment, the RG functional group is a hydrogen bond donor or acceptor group or atom for forming a hydrogen bond with a respective acceptor or donor group on a molecule of interest M;

wherein the L lipid scaffold backbone is represented by L1+L2+L3+L4+L5+L6 and wherein L comprises 5 to 40 carbon atoms or 5 to 30 carbon atoms or 5 to 25 carbon atoms or 5 to 20 carbon atoms and 0 to 2 cis or trans C═C double bonds;

wherein L1 is a carbon chain having 1 to 30 carbon atoms, 3 to 30 carbon atoms, 4 to 30 carbon atoms, 5 to 25 carbon atoms, 6 to 25 carbon atoms or 7 to 20 carbon atoms, and optionally L1 has one or more cis or trans C═C double bonds or 0 to 2 cis or trans C═C double bonds;

wherein L2 and L4 are each carbon atoms;

L3 is 0 to 20 carbon atoms and comprises 0 to 2 cis or trans C═C double bonds;

L5 is 0 to 20 carbon atoms and comprises 0 to 2 cis or trans C═C double bonds;

L6 is —CH₃, ═CH₂ or H;

each R is independently a linear or branched hydrocarbon chain having 0 to 30 carbon atoms and 0 to 2 cis or trans C═C double bonds, wherein according to one alternative embodiment, each R is independently branched with each branch point including an X2 functional group comprising a heteroatom;

wherein n is 0 to 8 and p is 0 to 8, and wherein n+p is ≥1 or 1 to 8 or wherein n is 0 to 6 and p is 0 to 6, and wherein n+p is ≥1 or 1 to 6 or wherein n is 0 to 4 and p is 0 to 4 and wherein n+p is a ≥1 or 1 to 4; and

X1 and X2 are independently selected from an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups including an alkane, alkene or alkyne, methylene (CH₂) or urea; or wherein X1 comprises one or more hydrogen bonds and has the structure of Formula V defined below.

In another embodiment, the lipid moiety of Formula IIa may be derived from precursor P having a structure of Formula IIIc:

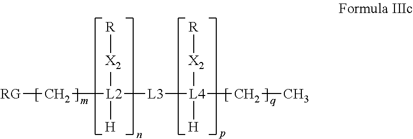

wherein RG is a reactive functional group comprising one or more reactive atoms selected from O, C, N, P, Si or B. In one embodiment, the reactive functional group is selected from a hydroxyl, amine or a carboxyl group. In another embodiment, the reactive functional group is a hydroxyl or carboxyl group. In an alternative embodiment, the RG functional group forms a biodegradable chemical linkage with a linker on a molecule of interest such as a drug;

wherein L is denoted by [CH₂]ₘ-L2-L3-L4-[CH₂]_{q}—CH₃, wherein the total number of carbon atoms in L is 5 to 30;

L2 and L4 are carbon atoms;

wherein m is 0 to 20; n is 1 to 4, p is 0 to 4, and n+p is 1 to 4;

L3 is 0 to 10 carbon atoms and has 0 to 2 cis or trans C═C;

X1 and X2 are independently selected from an ether, ester and carbamate group;

wherein each R is independently:
(a) a linear or branched terminating hydrocarbon chain with 0 to 5 cis or trans C═C and 1 to 30 carbon atoms and wherein each R is conjugated to one of a respective X2 at any carbon atom in its hydrocarbon chain thereof; or
(b) a branched structure of Formula IIb having a scaffold denoted by L′:

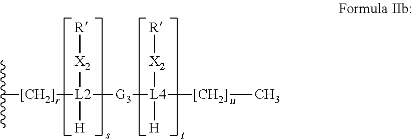

wherein L′ is denoted by [CH₂]ᵣ-L2-G₃-L4-[CH₂]ᵤ—CH₃, wherein the total number of carbon atoms in L is 3 to 30; and
wherein r is 0 to 20, 2 to 20, 3 to 20 or 4 to 20;
s is 0 to 4, t is 0 to 4; and wherein s+t is >1 or is 1 to 4;
u is 1 to 20;
G₃ is 0 to 10 carbon atoms and has 0 to 2 cis or trans C═C;
wherein each R′ of Formula IIb is independently a linear or branched terminating hydrocarbon chain with 0 to 5 cis or trans C═C and 1 to 30 carbon atoms;
wherein the total number of R′ hydrocarbon chains in Formula IIb is 1 to 16;

wherein each one of the R and R′ hydrocarbon chains in the lipid moiety is optionally substituted with a heteroatom, with the proviso that no more than 8 heteroatoms are substituted in the R and R′ hydrocarbon chains and wherein the predicted or experimental log P of the conjugate is greater than 5; and

wherein the lipid-conjugate is not an ionisable lipid.

### Molecule of Interest

A variety of molecules of interest may be linked to the lipid moiety. As noted, the lipid conjugate may be a pro-drug. The drug moiety of the pro-drug conjugate can be derived from any class of drug, including any drug used to treat, prevent, ameliorate, reduce the symptoms of and/or diagnose a disease or other undesirable condition in a subject, for instance after its activation. The drug moiety may be an active agent or an agent that is subsequently activated such as after its release from the conjugate. However, other molecules of interest can be linked to the lipid moiety as well, including hydrophilic polymers.

The molecule of interest M can be characterized in some embodiments by the nature of its attachment or association with the lipid moiety. For example, drug moiety D in certain embodiments may be derived from a drug that has lost one or more atoms upon its conjugation to a reactive group on scaffold L or to a linker group to form chemical linkage X1. In one embodiment, the drug loses a hydroxyl group or a hydrogen atom upon conjugation with P or a linker to form the pro-drug of Formula I, Ia, IIa or IIb. However, the drug moiety D may be derived from any known drug since the inventive methods described herein are applicable to the conjugation or association of a broad range of agents to the lipid moiety. The drug D may be a small molecule or a macro-molecular structure. The moiety M (molecule of interest) may be derived from a chemical structure that contains one or more reactive functional groups such as —(C═O)O, —OH, —NH₂, —NHR, —PO₃H₂, among others known to those of skill in the art, without limitation to the orientation of the atoms.

For example, the pro-drug or other lipid conjugate described herein may be formed (directly or via one or more intermediates) by a conjugation between a (C═O)OH group on the molecule of interest and a hydroxyl group on precursor scaffold P when RG is —OH. The general reaction is shown below:

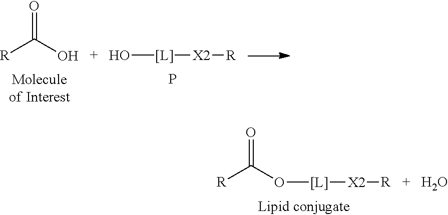

In the above exemplary embodiment, the X1 chemical linkage of Formula I, Ia, II or IIa is an ester and has the following structure:

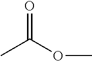

In another illustrative example, the molecule of interest M may have a hydroxyl group (—OH) that reacts with a carboxyl group ((C═O)OH) in a linker. A second carboxyl group ((C═O)OH) on the linker may react with a hydroxyl group on a carbon atom on a precursor scaffold P via a condensation reaction. The following reaction depicts the use of succinic acid as a linker. The use of such a linker results in a pro-drug that has two ester groups according to the following reaction:

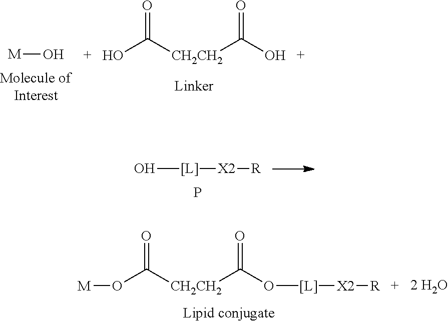

In the above non-limiting example, the X1 chemical linkage has the following structure;

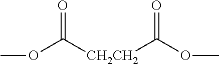

It should be appreciated that the above reaction may proceed in two steps. That is, the drug may first be conjugated to the linker and the resultant drug-linker conjugate subsequently reacted with the precursor scaffold P to produce a pro-drug reaction product.

As discussed below, the foregoing is provided simply for illustrative purposes as a variety of different linkers besides succinic acid can be used to produce the pro-drug. In another example, the molecule of interest M or a linker may have a carboxyl group ((C═O)O) for conjugation with an amine group of L to form an amide or amide-containing linkage X1 between the drug moiety and L. As discussed below, other reactions between functional groups on a drug or a linker with a scaffold L can be envisaged by those of skill in the art to produce an X1 chemical linkage.

Certain molecules of interest may comprise more than one reactive functional group for linkage to precursor scaffold P. In such embodiments, a protecting group may be employed during the synthesis of the drug-lipid conjugate as would be appreciated by those of skill in the art to selectively conjugate a given group on the drug to the scaffold L and leave another group unconjugated. The drug may also be characterized by its biological effect, including its ability to treat, prevent and/or ameliorate a condition in a subject or cells in vitro. The drug moiety may be derived from an anti-cancer agent, such as an anti-neoplastic agent. In another embodiment, the drug moiety may be derived from an immunomodulatory drug, such as an immunosuppressant, to treat an autoimmune disorder such as Crohn's disease, rheumatoid arthritis, psoriasis, ulcerative colitis or diabetes. In one embodiment, the immunomodulatory drug is an anti-inflammatory agent.

As used herein, a drug that functions as an anti-cancer agent may have a direct or an indirect effect on the growth, proliferation, invasiveness and/or survival of neoplastic cells and/or tumours. Anti-neoplastic drugs include alkylating agents, antimetabolites, cytotoxic antibiotics, various plant alkaloids and their derivatives and immunomodulatory agents.

Examples of immunosuppressant drug classes include glucocorticoids, cytostatics, antibodies, drugs acting on immunophilins, among others known to those of skill in the art. Examples of glucocorticoids include prednisone, prednisolone and dexamethasone. Methotrexate is an example of a cytostatic agent.

In one embodiment, the drug moiety is derived from docetaxel, dexamethasone, methotrexate, NPC1I, abiraterone, prednisone, prednisolone, ruxolitinib, tofacitinib, calcitriol, calcifediol, cholecalciferol, sirolimus, tacrolimus, acetylsalicylic acid, mycophenolate, cabazitaxel, betamethasone, and NLRP3 inhibitors, including CY09 (4-[[4-Oxo-2-thioxo-3-[[3-(trifluoromethyl)phenyl]methyl]-5-thiazolidinylidene]methyl]benzoic acid), INT-MA014 or MCC950 (N-(1,2,3,5,6,7-Hexahydro-s-indacen-4-ylcarbamoyl)-4-(2-hydroxy-2-propanyl)-2-furansulfonamide) and derivatives thereof, and cannabinoids, including cannabigerol, cannabichromene, cannabidiol, cannabidivarin, cannabicyclol, cannabicitran, cannabielsoin, cannabinol, tetrahydrocannabinol or tetrahydrocannabivarin and derivatives thereof.

In a further embodiment, the drug has a free hydroxyl group for conjugation to a linker or a group on any carbon of L. However, other functional groups on the drug could be used for such conjugation as well.

Other molecules of interest M besides drugs can be linked to the lipid moiety via X1 to scaffold L using similar reactive groups as those described above. This includes small molecules and those that form macro-molecular structures. For example, in some embodiments, the molecule of interest M is a polymer to form a lipid-polymer conjugate. The polymer may be a hydrophilic polymer suitable for use in biological systems. Examples of hydrophillic polymers include polyalkylethers, such as polyethylene glycol (PEG), polymethylethylene glycol, polypropylene glycol, and polyhydroxypropylene glycol.

Additional suitable polymers include polyvinylpyrrolidone, polyvinyl alcohol, polyacrylic acid, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose or polyaspartamide. The polymer chains may have a molecular weight of between about 300-10,000 daltons. The polymer may be a block co-polymer in certain non-limiting embodiments.

In yet further embodiments, the molecule of interest M is an antibody, peptide, genetic material, such as siRNA.

In one embodiment, the molecule of interest M is genetic material, such as a nucleic acid. The nucleic acid includes, without limitation, RNA, including small interfering RNA (siRNA), small nuclear RNA (snRNA), micro RNA (miRNA), or DNA such as plasmid DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one particularly advantageous embodiment, the molecule of interest is an siRNA. An siRNA becomes incorporated into endogenous cellular machineries to result in mRNA breakdown, thereby preventing transcription. Since RNA is easily degraded, its incorporation into a delivery vehicle as described herein can reduce or prevent such degradation, thereby facilitating delivery to a target site.

### Chemical Linkage X1

In one embodiment, the molecule of interest M is directly linked to the L scaffold carbon chain via an X1 functional group. In such embodiment, X1 in Formula I, Ia, II or IIa may be one or more functional group selected from an ester, amide, amidine, hydrazone, disulfide, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups such as an alkane, alkene or alkyne, methylene (CH₂) or urea.

In one embodiment, the X1 group is not a disulfide or thioether. In another embodiment, X1 does not contain a sulfur atom.

As discussed, the molecule of interest M can be attached to the L scaffold via an X1 that is a linker. The inclusion of a linker group in the lipid conjugate is particularly advantageous for those molecules that are released from the lipid moiety after administration, such as for example, a pro-drug, as its inclusion can facilitate cleavage of the molecule of interest M from the lipid moiety by an enzyme. As described below, one or more of the foregoing functional groups, including but not limited to those specifically depicted in Table 3 below, can be included in the linker molecule. Such functional group most advantageously can be cleaved under in vivo conditions.

Non-limiting examples of lipid conjugates having X1 chemical linkages selected from a succinic acid linker, ester, amide, hydrazone, ether, carbamate, carbonate or phosphodiester group are depicted below in Table 3. The chemical linkages below are shown as part of Formula I or Formula Ia. As discussed, although the linkages are depicted as those produced by direct conjugation between a drug and L for simplicity (apart from succinate), it will be appreciated that the groups shown in the table can also be incorporated within a linker group.

TABLE 3
Examples of lipid conjugates having various functional groups forming the XI chemical linkages

| X1 | | | |
|---|---|---|---|
| chemical | | | |
| linkage | Formula I | Formula Ib | Formula II |
| succinate linker | 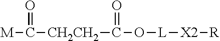 | 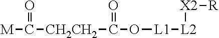 | 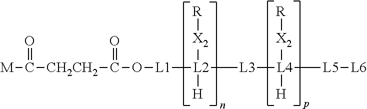 |
| ester | 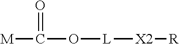 | 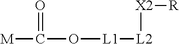 | 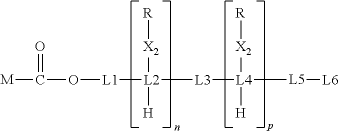 |
| amide | 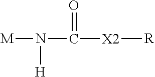 | 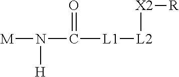 | 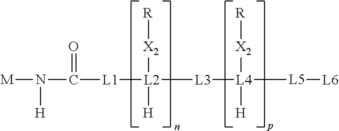 |
| hydrazone | 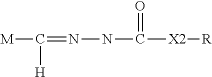 | 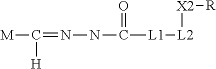 | 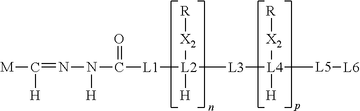 |
| ether | 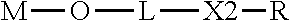 | 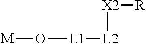 | 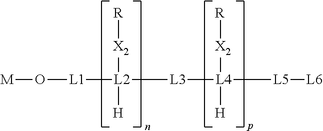 |
| carbamate | 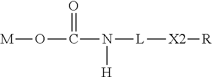 | 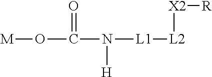 | 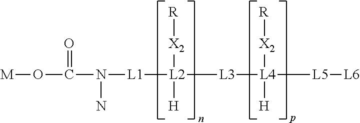 |
| carbonate | 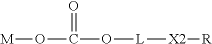 | 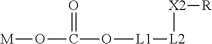 | 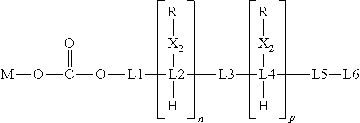 |
| phosphodi- ester | 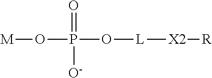 | 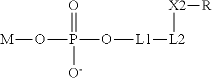 | 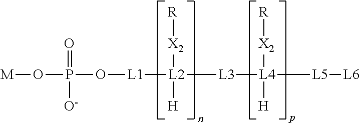 |

As set forth above, in certain advantageous embodiments, a hydroxyl group of a precursor scaffold P (RG=OH in Formula III, IIIa, III or IIIc) or an amine of a precursor scaffold P (RG=NH₂ in Formula III, IIIa, IIb or IIIc) reacts with a carboxyl group on a drug to form an X1 chemical linkage that is an ester or amide group, respectively, via a condensation reaction.

In such embodiments X1 of the lipid conjugate of Formula I, Ia, II or IIa has the following structure:

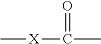

wherein X═—O, or —NH.

In such embodiment, the X1 chemical linkage forms part of the pro-drug of Formula I as follows:

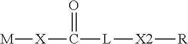

In an alternative embodiment, L is derived from reaction of a carboxyl group of the fatty acid with a hydroxyl or amine group of a linker or a molecule of interest. In this embodiment, X1 forms the following chemical linkage between a molecule of interest and P:

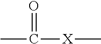

wherein X═—O, or —NH.

In one particularly advantageous embodiment, X═—O in the foregoing structures. In such embodiment, X1 is an ester bond.

In one embodiment, the X linkage is biodegradable, meaning that it can be cleaved after administration to a patient. Without being limiting, an ester bond is capable of being hydrolyzed by an esterase after administration to a patient, thereby releasing a molecule of interest, including but not limited to a drug moiety D, from the lipid conjugate. However, other X1 linkages can be utilized for tailored drug release based on their release characteristics when exposed to the environment at a disease site. For example, a hydrazone bond positioned between drug moiety D and scaffold L can impart pH sensitive release to the conjugate of Formula I, Ia, II or IIa. At neutral pH, hydrazones may exhibit little to no decomposition, while at a lower pH the bond may be broken. Thus, an X1 chemical linkage consisting of, or that comprises, one or more hydrazone bonds can provide for drug release at the low pH values often present in tumor tissues.

In one embodiment, X1 is cleavable by an esterase, alkaline phosphatase, amidase, peptidase or may be cleavable upon exposure to a reducing environment, and/or a high or low pH.

As discussed, if the lipid conjugate is a pro-drug, the X1 chemical linkage in certain embodiments is most advantageously a linker. A wide variety of chemical linkers is known to those of skill in the art and may be employed in certain embodiments described herein. A linker may have 0 to 12 carbon atoms and at least one cleavable functional group. In one embodiment, the linker has at least two functional groups, a first functional group for conjugating one end of the linker to molecule of interest M and a second functional group for conjugating another end of the linker to a carbon atom on L. The two functional groups may each be independently selected from an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups such as an alkane, alkene or alkyne, methylene (CH₂) or urea.

As would be appreciated by those of skill in the art, in some embodiments, if the molecule of interest is a drug D, a linker may provide enhanced release of the drug D through the introduction of a biodegradable group. A linker having one or more ester bonds may be capable of being hydrolyzed by an esterase after administration to a patient, thereby releasing drug moiety D from the pro-drug conjugate. Similar to a linkage resulting from direct reaction between D and L, a linker introducing a hydrazone bond between drug moiety D and scaffold L can impart pH sensitive release to the pro-drug of Formula I or Ia.

However, it will be understood that the foregoing is merely exemplary. Additional examples of linkers are provided in U.S. Pat. No. 5,149,794, which is incorporated herein by reference. Non-limiting examples of linkers described in U.S. Pat. No. 5,149,794 include aminohexanoic acid, polyglycine, polyamides, polyethylenes, and short functionalized polymers having a carbon backbone that is one to twelve carbon atoms in length.

Yet further examples of linkers suitable for use in the pro-drugs described herein are provided in the following references:
1. Rautio et al., “The expanding role of prodrugs in contemporary drug design and development” Nature Reviews Drug Discovery 2018, 17, 559.
2. Irby et al., “Lipid-drug conjugate for enhancing drug delivery” Molecular Pharmaceutics 2017, 14, 1325.
3. Sun et al., “Chemotherapy agent-unsaturated fatty acid prodrugs and prodrug-nanoplatforms for cancer chemotherapy” Journal of Controlled Release 2017, 264, 145.
4. Walther et al., “Prodrugs in medicinal chemistry and enzyme prodrug therapies” Advanced Drug Delivery Reviews 2017, 118, 65.
5. Hu et al., “Glyceride-mimetic prodrugs incorporating self-immolative spacers promote lymphatic transport, avoid first-pass metabolism and enhance oral bioavailability” Angewandte Chemie International Edition 2016, 55, 13700.
6. Blencowe et al., “Self-immolative linkers in polymeric delivery systems” Polymer Chemistry 2011, 2, 773.

Each of the foregoing references is incorporated herein by reference in its entirety. In further embodiments, the X1 chemical linkage comprises both a functional group and a separate linker. Various combinations of linkers and functional groups (such as those in Table 3 above) can be employed to attain a desired lipid conjugate of Formula I, Ia, II or IIa.

In one embodiment, at least the second functional group conjugating one end of the linker to L1 is an ester or an amide linkage. In another embodiment, a functional group on the linker can be hydrolyzed by an enzyme such as an esterase. In a further embodiment, both functional groups on the linker are ester linkages.

While a broad range of known linkers can be utilized in embodiments described herein, some non-limiting examples of formulas for X1 linkers are provided below.

In one embodiment, without being limiting, the molecule of interest M-linker X1(D-X1) portion of Formula I, Ia, II or IIa has the Formula IV below:

M-[X4-M₁-X5]_{X2} Formula IV:

wherein M is the molecule of interest, X4 and X5 are independently selected from any functional group described previously and M₁ is an optional spacer group linked to the X4 and X5 functional groups and has 0 to 12 carbon atoms or is CH₂, CH₂CH₂, N-alkyl, N-acyl, O or S. X4 and X5 can be the same or different. In one embodiment, either or both of X4 and X5 are capable of being cleaved in vivo, in another embodiment, X4 and/or X5 is an ester group.

The X4, X5 or both functional groups in Formula IV above individually can be repeating units of 1 to about 20. Moreover, the X4-M₁-X5 unit can be a repeating unit of 1 to 20 or X4-X5 can be a repeating unit if no M₁ is present.

In a further embodiment, X5 in Formula IV is an ester group, in which case M-X1 of Formula I, Ia, II or IIb is as follows:

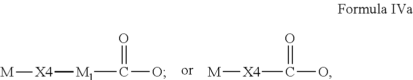

wherein M is a molecule of interest and X4 is a functional group that covalently links M to M₁ and is selected from an ester, amide, hydrazone, ether, carbonate, carbamate or phosphodiester group; and M₁ is a spacer region of the linker having 0 to 12 carbon atoms or is CH₂, CH₂CH₂, N-alkyl, N-acyl or O.

In one embodiment, without being limiting, the linker X2 of Formula I, Ia, II or IIa has the structure below:

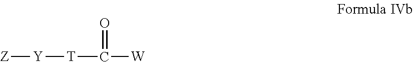

wherein Z is selected from 0 or N, Y is CH₂, CH₂CH₂ or C═O, T is 0 to 6 carbon atoms and W is O or N. In one embodiment, Z is 0, Y is CH₂, CH₂CH₂ or C═O, T is 0 to 6 carbon atoms and W is 0. In a further embodiment, linker X1 is derived from succinic acid.

In such embodiment, the linker of Formula IVb forms part of the lipid conjugate of Formula I, Ia and II as follows:

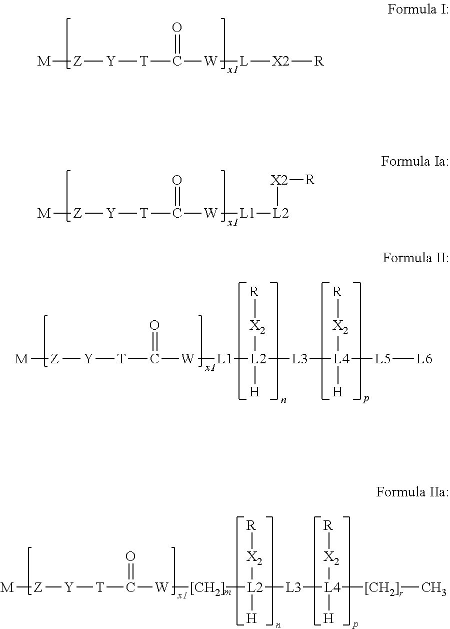

wherein Z is selected from 0 or N, Y is CH₂, CH₂CH₂ or C═O, T is 0 to 6 carbon atoms and W is O or N. In one embodiment, Z is 0, Y is CH₂, CH₂CH₂ or C═O, T is 0 to 6 carbon atoms and W is 0. In a further embodiment, linker X1 is derived from succinic acid.

In one particularly advantageous embodiment, the X1 linker is a succinate group and the pro-drugs of Formula I, Ia, II and IIa have the structures shown below:

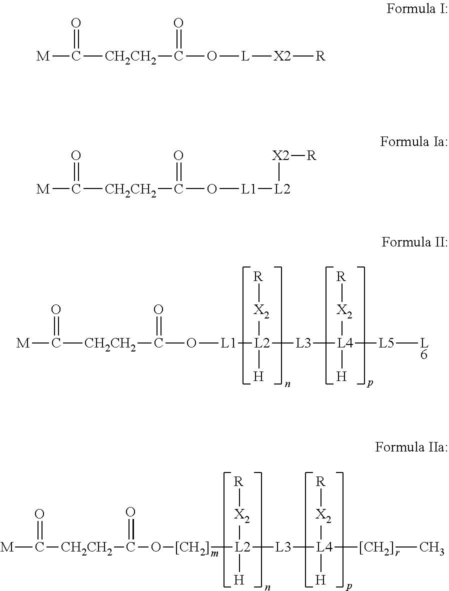

Non-limiting examples of X1 linkages besides a succinic acid linker include the following chemical structures:

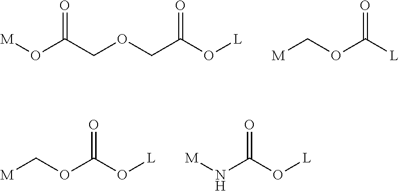

wherein M is a molecule of interest and L is the lipid scaffold. For simplicity, the remainder of the lipid moiety is not shown in the foregoing structures, but can include any lipid moiety of Formula I, Ia, II and IIb.

It should be understood that the reactions to produce the X1 chemical linkage are not limited to those that result from the direct reaction between respective functional groups present on the molecule of interest, such as a drug, polymer or linker attached thereto and a corresponding group on the precursor scaffold P. Typically, such conjugates are produced by synthesis schemes that are multi-step and proceed through various intermediates. Moreover, it is possible to modify a precursor L, such as a fatty alcohol to produce derivatives thereof and the derivatives can, in turn, be reacted with a reactive functional group on a molecule of interest to produce a lipid conjugate or vice versa. For example, US 2002/0177609 (incorporated herein by reference) describes methods that involve derivatizing a fatty alcohol with an appropriate linkage and leaving group to form an intermediate and reacting the intermediate with a drug to form a conjugate compound. A number of different X1 linkages can be produced in this manner including a drug conjugated to scaffold L via one or more carbonate, carbamate, ether, phosphate, ester, guanidine, thionocarbamate, phosphonate, oxime, isourea, amide, phosphoramide, or phosphonamide groups. Likewise, it is possible to modify other molecules besides fatty alcohols to introduce reactive groups that cannot be produced by reacting existing functional groups present on the drug and a fatty acid.

In further embodiments, the molecule of interest M is linked to scaffold L of the lipid moiety via an X1 linkage comprising one or more intermolecular hydrogen bonds. According to such embodiment, the molecule of interest comprises one or more electronegative atoms. The molecule of interest may comprise at least one hydrogen bond donor, which is a hydrogen atom covalently attached to a relatively electronegative atom and L may comprise at least one hydrogen bond acceptor, which is a relatively electronegative atom bonded to the hydrogen by the hydrogen bond. Conversely, L may comprise one or more hydrogen bond donor and the molecule of interest M may comprise one or more hydrogen bond acceptor.

The hydrogen bond between L and M of the lipid conjugate may have the structure of Formula V:

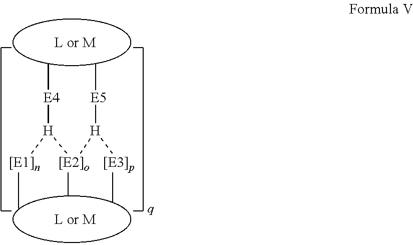

wherein E1, E2, E3, E4 and E5 are electronegative atoms selected from O, N and P;

E1, E2 and E3 are hydrogen bond acceptors and E4 and E5 are hydrogen bond donors;

the dotted lines depict hydrogen bonds and the solid lines depict covalent bonds;

wherein L is a lipid scaffold of the lipid moiety as set forth in Formula I, Ia, II or IIa;

n is 0 or 1; o is 0 or 1; and p is 0 or 1; and wherein n+o+p≥2;

q is 1 to 10 or 2 to 10 or 4 to 10;

L is a lipid scaffold of the lipid moiety;

M is a molecule of interest; and

wherein E1 and E3 optionally comprise substituents linked thereto such as an alkyl, aryl, alkylene or H.

Examples of drug-lipid conjugates comprise X1 hydrogen bond linkages are provided below. In this example doxorubicin comprises hydrogen bond acceptor groups and a lipid moiety with a terminal

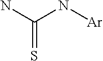

group comprises hydrogen bond donor groups, it will be understood, however, that other atomic configurations of hydrogen bond donors and acceptors could be readily envisaged by those of ordinary skill in the art.

Example of a hydrogen bond X1 linkage in the drug-lipid conjugate:

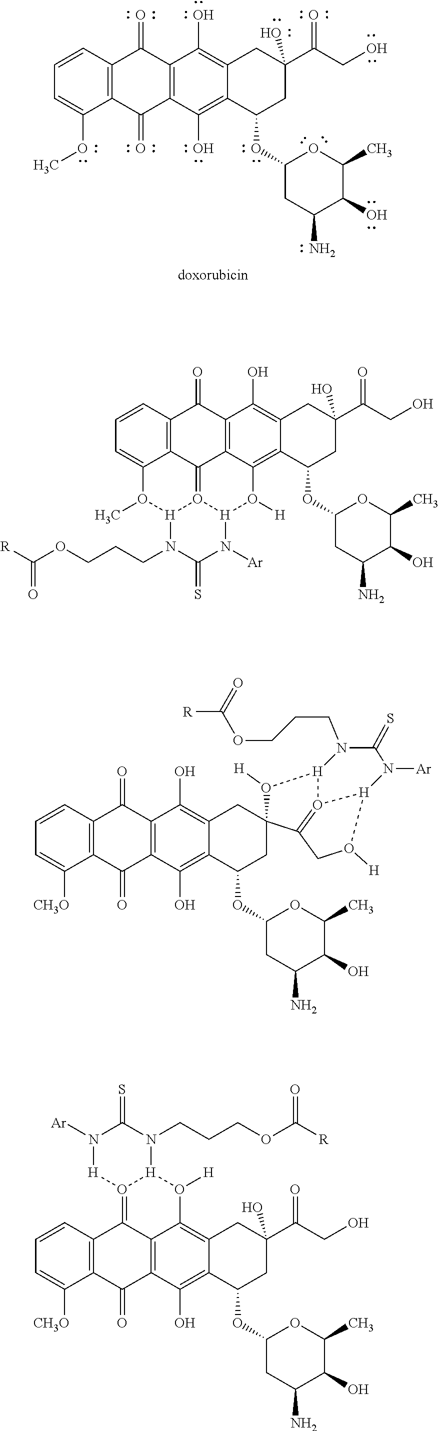

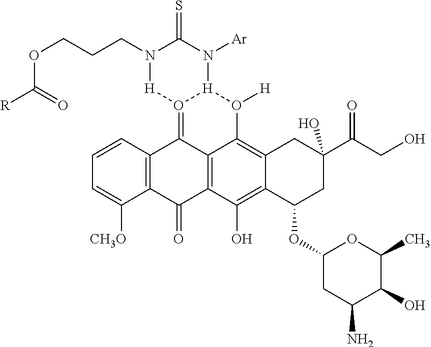

Chemical Linkage X2

Likewise, X2 is a chemical linkage that covalently links R to any carbon atom on L of Formula I, Ia, II or IIa and may be formed by reaction of a functional group on any carbon of L with a reactive group on R. Similar to X1, however, X2 need not result from direct reaction between a functional group on L but rather can be formed by a multi-step synthesis scheme.

Various X2 functional groups can link R to L or 12. For example, X2 may be a functional group selected from an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups such as an alkane, alkene or alkyne, methylene (CH₂) or urea. In one embodiment, the reactive group on L to form X2 with the reactive group on R is a functional group selected from an —OH, an —NH₂, or a —C═O(O). Most advantageously, X2 is —C═O(O) that is formed via reaction of an acyl group with a hydroxyl group on L. Such groups, however, are merely exemplary and other groups known to those of skill in the art could be employed as well.

The X2 chemical linkage may also be a linker. A linker may have 0 to 12 carbon atoms and at least one cleavable functional group to release R in Formula I, Ib, II or IIa if desired. In one embodiment, the linker has at least two functional groups, a first functional group conjugating one end of the linker to scaffold L and a second functional group conjugating another end of the linker to a carbon atom on R. The two functional groups may each be independently selected from an ester, amide, amidine, hydrazone, disulfide, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo, carbon-based functional groups such as an alkane, alkene or alkyne, methylene (CH₂) or urea. In one advantageous embodiment, at least one of the functional groups in the linker is an ester, amide, hydrazone, ether, carbonate, carbamate or phosphodiester. In another embodiment, at least one of the functional groups of X2 can be cleaved in vivo to release R from scaffold L. Such latter embodiment may be desirable if R or L is a therapeutic lipid.

### R or R′ Group

As noted, in one embodiment, R or R′ in Formula I, Ia, II or IIa is a hydrocarbon group with 1 to 40 carbon atoms, and optionally has one or more cis or trans C═C double bonds. In another embodiment, R is an aliphatic hydrocarbon. In a further embodiment, R does not comprise any heterocyclic ring structures. In another embodiment, R is not biotin.

In one embodiment, the number of carbon atoms in the R group is selected so that the lipid conjugate of Formula I, Ia, II or IIa has a desired Log P value. As can be seen from Table 1 above, in some embodiments, the log P of the lipid conjugate may generally be correlated with the number of carbon atoms on hydrocarbon R. For instance, in the example provided in Table 1 based on L (Formula I) or L1-L2 (Formula Ia) derived from ricinoleyl alcohol, if the R hydrocarbon is derived from an acyl group having 2 carbon atoms as in INT-D047 (i.e., R is 1 carbon atom based on Formula I or Ia nomenclature described above), then the Log P is only 8.33. However, the Log P of INT-D048 derived from an acyl chain of 5 carbon atoms is 10.13 (i.e., 5 is 4 carbon atoms based on Formula I or Ia S nomenclature). The Log P of INT-0035 Increases to 15.34 when oleoyl having 18 carbon atoms is conjugated to L (i.e., R is 17 carbon atoms based on Formula I or Ia R nomenclature). When the acyl chain conjugated to L has 20 carbon atoms as in INT-D051, then Log P is 15.14 (i.e., S equals 19 carbon atoms). As discussed, by designing a pro-drug to possess a desired hydrophobicity, drug loading and retention properties after administration can be more easily controlled.

Thus, in one embodiment, R in Formula I, Ia, II or IIa has 1 to 40 carbon atoms and is linear or branched and is selected to provide the lipid conjugate with a desired log P falling within the range of 5 to 25 or 5 to 18 or 6 to 16.

As discussed, optionally a second R hydrocarbon with 1 to 40 carbon atoms, and optionally having one or more, cis or trans C═C double bonds is chemically linked to L via an X2 chemical linkage. Yet further, optionally, a third R hydrocarbon with 1 to 40 carbon atoms, and optionally having one or more, cis or trans C═C double bonds is chemically linked to L via an X2 chemical linkage.

Moreover, one or more R hydrocarbon moieties linked to L may have linked thereto a respective R′ side chain. For example, an R′ side chain may be linked to a first, second or third R via an X2 linkage and a second R′ side chain may be linked to any R via an X2 linkage and/or a third R′ may be linked to any R via an X2 linkage. Various other combinations could be readily envisioned by those of skill in the art.

It should be appreciated that the R hydrocarbon need not be derived from an acyl group or a fatty acid. For example, R could be a cholesterol moiety or other hydrocarbon group. The R hydrocarbon could also be a therapeutic or prophylactic moiety that is released upon its cleavage from the pro-drug, such as a lipid or sterol having therapeutic activity.

As noted, a variety of chemical linkages can be utilized to link the molecule of interest M to L, one or more R hydrocarbons to L or one or more R′ groups to R. It will be appreciated by those of skill in the art that various functional groups or combinations thereof can be employed in these linkages. That is, X1 and X2 described above in various embodiments above in connection with the lipid conjugates of Formula I, Ia, II and IIa and precursor P of Formula III, IIIa, IIIb and IIIc can be independently selected from an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyaikylamine, sulfonamide, imine, azo, carbon-based functional groups such as an alkane, alkene or alkyne, methylene (CH₂) or urea. In a further alternate embodiment, any one of linkage X1 and X2 is biodegradable.

In a further embodiment, any X2 is a linkage comprising one or more hydrogen bonds. According to such embodiment, X2 will have the structure of the linking portion of Formula VI:

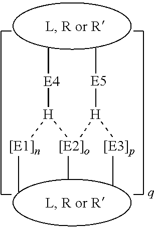

wherein E1, E2, E3, E4 and E5 are electronegative atoms selected from O, N and P;

E1, E2 and E3 are hydrogen bond acceptors and E4 and E5 are hydrogen bond donors;

the dotted lines depict hydrogen bonds and the solid lines depict covalent bonds;

wherein L is a lipid scaffold of the lipid moiety as set forth in Formula I, Ia, II or IIa;

R and R′ are hydrocarbon chains as set forth in Formula IIa;

n is 0 or 1; o is 0 or 1; and p is 0 or 1; and wherein n+o+p≥2;

g is 1 to 10 or 2 to 10 or 4 to 10;

L is a lipid scaffold of the lipid moiety;

M is a molecule of interest; and

wherein E1 and E3 optionally comprise substituents linked thereto such as an alkyl, aryl, alkylene or H.

Products, Compositions and Formulations

The lipid conjugates described herein can be administered in either free form, including as a component of a pharmaceutical product or composition, or as part of a delivery vehicle. Such products or compositions typically include known pharmaceutically acceptable salts and/or excipients.

A variety of delivery systems can be used to prepare pharmaceutical formulations. These include but are not limited to nanoparticles (LNPs), including lipid nanoparticles including vesicles with one or more bilayers such as liposomes or polymer nanoparticles comprising lipids, polymer-based nanoparticles, emulsions, micelles, and carbon nanotubes.

The lipid conjugates of the present disclosure are particularly amenable to incorporation into nanoparticles, such as liposomes or polymer-based systems comprising lipids or other hydrophobic components. The lipid-like properties of the lipid conjugate in certain embodiments may facilitate its loading into these or other delivery vehicles. For example, in some embodiments, the loading efficiency into a given nanoparticle is 75% to 100%, 80% to 100% or most advantageously 90% to 100%.

In one embodiment, the lipid conjugates are loaded into lipid nanoparticles, such as liposomes, by mixing them with lipid formulation components, including vesicle forming lipids and optionally a sterol. As a result, lipid nanoparticles incorporating these drug-lipid conjugates can be prepared using a wide variety of well described formulation methodologies known to those of skill in the art, including but not limited to extrusion, ethanol injection and in-line mixing. Such methods are described in Maclachlan, I. and P. Cullis, “Diffusible-PEG-lipid Stabilized Plasmid Lipid Particles”, Adv. Genet., 2005. 53PA:157-188; Jeffs, L B., et al., “A Scalable, Extrusion-free Method for Efficient Liposomal Encapsulation of Plasmid DNA”, Pharm Res, 2005, 22(3):362-72; and Leung, A. K., et al., “Lipid Nanoparticles Containing siRNA Synthesized by Microfluidic Mixing Exhibit an Electron-Dense Nanostructured Core”, The Journal of Physical Chemistry. C, Nanomaterials and interfaces, 2012, 116(34): 18440-18450, each of which is incorporated herein by reference in its entirety.

While liposomes comprise an aqueous internal solution surrounded by a phospholipid bilayer, a lipid nanoparticle may alternatively comprise a lipophilic core. Such lipophilic core can serve as a reservoir for the pro-drug. Solid and liquid lipid nanoparticles can be used for the delivery of the pro-drugs as described herein.

Provided in one embodiment is a lipid nanoparticle that comprises a phospholipid bilayer and wherein the lipid conjugate forms a hydrophobic oil phase within the bilayer. Such delivery vehicles are described in Example 3 and Example 4 herein. The hydrophobic oil phase can be visualized by electron microscopy. In one embodiment, the lipid conjugate has the structure of Formula I, Ia, II or IIa. In another embodiment, the lipid nanoparticle is a particle with one or more bilayers such as a liposome.

The delivery vehicle can also be a nanoparticle that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid. In such embodiments, the lipid conjugate of the disclosure may be a lipid-polymer conjugate.

Nanoparticles may alternatively be prepared from polymers without lipids. Such nanoparticles may comprise a concentrated core of drug that is surrounded by a polymeric shell or may have a solid or a liquid dispersed throughout a polymer matrix.

The lipid conjugates described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

Micelles are self-assembling particles composed of amphipathic lipids or polymeric components that are utilized for the delivery of agents present in the hydrophobic core. Conjugating a drug to a scaffold molecule L and with a hydrophobic group R as described herein may improve drug loading into a micelle.

A further class of drug delivery vehicles known to those of skill in the art that can be used to encapsulate the lipid conjugate herein is carbon nanotubes.

Various methods for the preparation of the foregoing delivery vehicles and the incorporation of pro-drugs therein are available and may be carried out with ease by those skilled in the art.

Certain lipid conjugates encompassed by the disclosure may form part of a carrier-free system. In such embodiments, the lipid conjugate can self-assemble into particles. Without being limiting, if the drug moiety D or polymer is hydrophilic, then the amphiphilic pro-drug may assemble into nanoparticles with or without a stabilizer.

While pharmaceutical compositions are described above, the lipid conjugate can be a component of any nutritional, cosmetic, cleaning or foodstuff product.

Administration

In certain embodiments, the lipid conjugate is a pro-drug that is either free or formulated in a drug delivery vehicle and is administered to treat, prevent and/or ameliorate a condition in a patient. That is, the pro-drug in free form or formulated in a delivery vehicle may provide a prophylactic (preventive), ameliorative or a therapeutic benefit. A pharmaceutical composition comprising the pro-drug will be administered at any suitable dosage. In one embodiment, the pro-drug that is free or formulated in a drug delivery vehicle is administered parentally, i.e., intra-arterially, intravenously, subcutaneously or intramuscularly. In other embodiments, the pro-drug in free form or formulated in a delivery vehicle described herein may be administered topically. In still further alternative embodiments, the pro-drug in free form or formulated in a delivery vehicle described herein may be administered orally. In yet a further embodiment, the pro-drug in free form or formulated in a delivery vehicle are for pulmonary administration by aerosol or powder dispersion.

In further embodiments, the molecule of interest is a hydrophilic polymer and the conjugate is a lipid-polymer conjugate. The lipid-polymer conjugate may be incorporated into a delivery vehicle together with one or more drugs and administered to treat, prevent and/or ameliorate a condition in a patient.

The term patient used herein includes a human or a non-human subject.

The following examples are given for the purpose of illustration only and not by way of limitation on the scope of the invention.

### EXAMPLES

### Example 1: Ricinolyl-Alcohol as an Exemplary Scaffold Molecule L

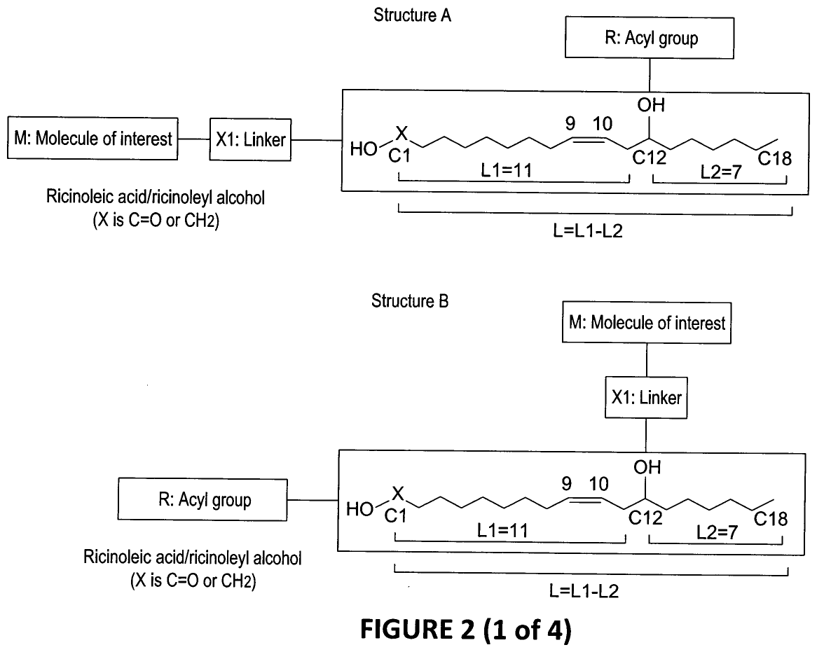

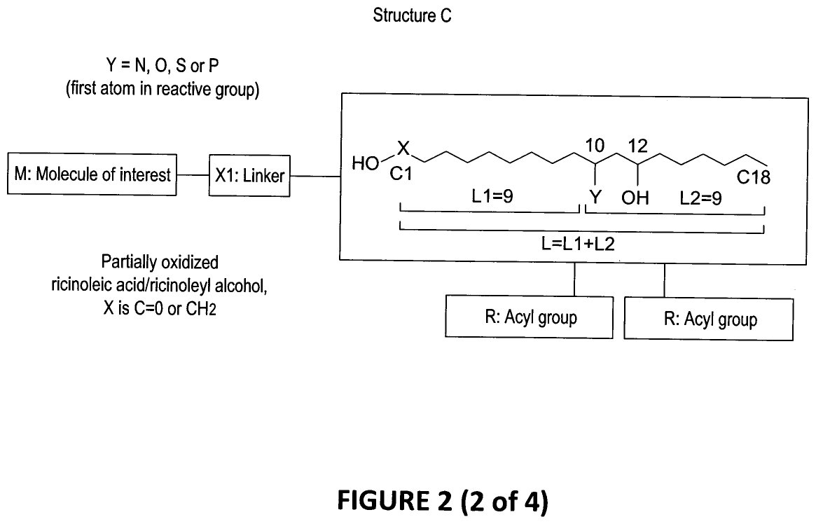

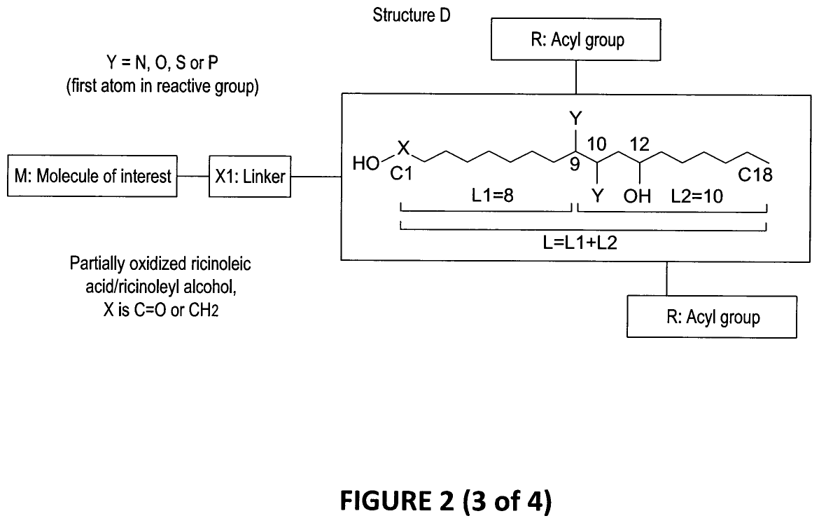

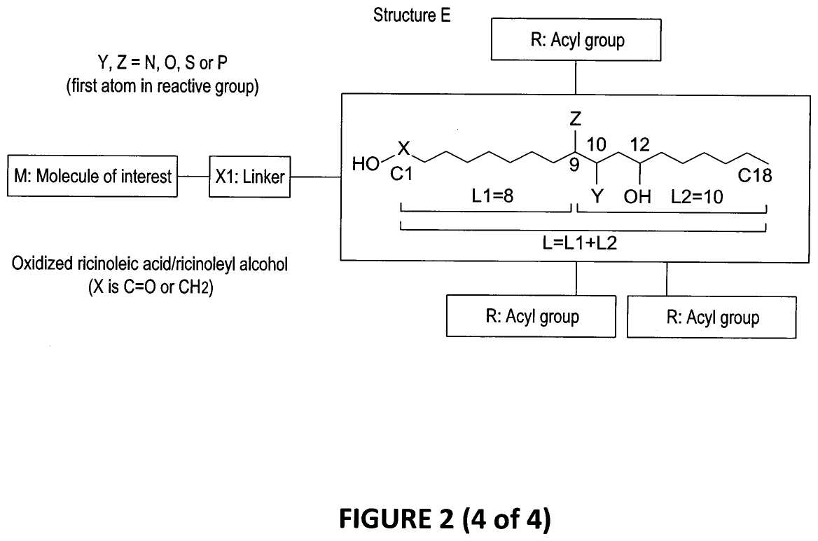

Examples of lipid conjugates are set forth in FIG. 2 and demonstrate the diversity of conjugates that can be formed using ricinoleic acid or ricinoleyl alcohol as a precursor for scaffold L in Formula I, Ia, II or IIa above. In the diagrams, the chemical structures of the X1 and X2 linkages of Formula I are not depicted. Rather, the diagrams show the hydroxyl groups at C1 and C12 of the fatty acid or alcohol (as well as atoms in Z, Y at positions 9 and 10 In an oxidized form of the molecule) that can be reacted with a complementary functional group on a drug-linker and/or acyl group, such as a carboxylic acid. In this example, the X1 and X2 linkages would comprise an ester functional group based on a condensation reaction between a carboxyl and a hydroxyl group, although other functional groups could form as well depending on the particular functional groups present on the drug, molecular scaffold, side group R or the linker group that react to form X1 or X2.

The examples below also employ a linker group to link the molecule of interest M to the scaffold molecule L. It should be appreciated, however, that such linker is optional in that the molecule of interest M alternatively can be conjugated directly to the scaffold molecule L itself.

Ricinolyl alcohol is an unsaturated fatty alcohol derived from ricinoleic acid, which is a hydroxyl fatty acid (HFA) having 18 carbon atoms and is substituted at C12 with a hydroxyl group. While in the structures of FIG. 2, ricinoleic acid or ricinoleyl alcohol is depicted as the precursor scaffold P (X is C═O or CHI), other molecules can be employed as well, including without limitation, other hydroxy fatty acids, their corresponding fatty alcohols or fatty amines. Moreover, a scaffold L based on ricinoleic acid or ricinoleyl alcohol need not be prepared from a fatty acid or a fatty alcohol having a hydroxyl both at C1 at C12. For example, a precursor for L can be prepared from a corresponding molecule having a hydroxyl at C1 and an ether substituent at C12 (such as the silyl ether I-1-(tert-Butyldimethylsilyl)-12-hydroxyoleyl alcohol (2) intermediate described in Example 2).

In some embodiments, the double bond of the backbone of ricinoleic acid or ricinoleyl alcohol is partially or fully oxidized to provide for an additional reactive group that can be used to conjugate a second acyl chain R′. Such groups are depicted as Y and Z in the drawings.

In this example, scaffold molecule L is described as an 11-12 chain of Formula Ia. L1 is the carbon chain from C2 to a carbon preceding a first branch point in which a side group (e.g., an acyl chain) or a molecule of interest or an M-inker is conjugated. L2 is the carbon chain including the carbon at the branch point to the terminal end of the scaffold.

In structure A of FIG. 2, X1 is a linker that covalently attaches the molecule of interest to ricinoleic acid (X═C═O) or ricinoleyl alcohol L (X═CH₂) at C1. The linker is attached to C1 of ricinoleic add or ricinoleylalcohol via a reactive group that is a hydroxyl group at C1. At C12 of ricinoleic acid or ricinoleyl alcohol, a side chain R, which is derived from an acyl group, is attached to L2 via a hydroxyl group. In this example, L is a linear, 11 carbon chain with a cis double bond as depicted in FIG. 2 at C9 and C10 and L2 is a 7 carbon saturated carbon chain from C12 to C18. X2 is not shown, but links C12 of ricinoleic acid or ricinoleyl alcohol to the R side chain, which is derived from an acyl group. As discussed above, the carboxylic acid of the acyl group reacts with the hydroxyl group at C12 of ricinoleic acid or ricinoleyl alcohol to form an —O(C═O) ester linkage. Likewise, in this example, the OH at C1 of ricinoleic acid or ricinoleyl-alcohol reacts with a carboxylic acid group at one end of the linker to form an X1-O(C═O) ester linkage. Alternatively, the OH at C1 of L reacts directly with a free carboxylic acid on a molecule of interest M to form an —O(C═O) linkage (X1).

In structure B of FIG. 2, a linker X1 covalently attaches the molecule of interest M to the hydroxyl group at C12 of ricinoleic acid or ricinoleyl alcohol. At C1 of the molecule, an R hydrocarbon derived from an acyl side group is attached to L1 via the terminal hydroxyl group at C1. In this example, L1 of the molecular scaffold is 11 carbon atoms and L2 is 7 carbon atoms. Alternatively, the OH at C1 of L reacts directly with a carboxylic acid on molecule of interest M to form an —O(C═O) linkage.

In structure C of FIG. 2, partially oxidized ricinoleic acid or ricinoleyl alcohol is used as the precursor scaffold P. The double bond of ricinoleic acid or ricinoleyl-alcohol at C9 and C10 is oxidized to produce a saturated hydrocarbon chain substituted at position C10 with a γ reactive group and C12 with a hydroxyl group. A side chain R derived from an acyl group is conjugated to C12 of 1.1-12 via the hydroxyl group and a second side chain R′ derived from another acyl chain is conjugated to the C10 position via Y. In this example, Y is a reactive group and comprises N, O, S or P as a first atom in the group. Without being limiting, if Y is N, then the reactive group may be an amine, if Y is O, then the reactive group may be a hydroxyl. Likewise, if Y is P, then the reactive group may be a phosphate. As discussed, these reactive groups are merely exemplary and other groups could easily be envisaged by those of skill in the art.

The molecule of interest M-linker X1 is attached at C1 via a terminal hydroxyl group of ricinoleic acid or ricinoleyl alcohol. Alternatively, the OH at C1 of 1-12 reacts with a carboxylic acid on molecule of interest M itself to form an —O(C═O) linkage. In this example, L is 9 carbon atoms and L2 is 9 carbon atoms.

In the structure D of FIG. 2, partially oxidized ricinoleic acid or ricinoleyl alcohol is again used as a scaffold precursor, and comprises molecule of interest M attached via linker X1 on C1. Alternatively, the OH at C2 of L1-L2 reacts directly with a carboxylic acid on molecule of interest M to form an —O(C═O) linkage rather than utilizing a linker as depicted. A first side chain R derived from an acyl chain is linked at the C12 position via the hydroxyl reactive group and a second side chain R′ derived from an acyl chain is attached at C9 of ricinoleyl-alcohol via a Y group, in which the first atom in the group is N, O, 5 or P as described in connection with Structure C. In this example, L1 is carbon atoms and L2 is 10 carbon atoms.

in the structure E of FIG. 2, oxidized ricinoleic acid or ricinoleyl alcohol is used as a precursor to scaffold L with a side chain derived from an acyl chain R linked at the C12 position via a hydroxyl group and a second side chain R′ derived from an acyl chain attached at C9 via a Z group. Similar to Y, the Z group is a reactive group, in which the first atom in the group is N, O, S or P as described in connection with Structure C and D above. A drug moiety D is attached via linker X1 on C1 by a chemical linkage formed with the reactive hydroxyl group at C1. Alternatively, the OH at C1 of L1-L2 reacts directly with a carboxylic acid on drug D to form an —O(C═O) linkage rather than utilizing a linker.

### Example 2: Synthesis of Livid Conjugates

Materials and Methods:

Various pro-drugs were prepared using the synthesis procedures A-E set forth below.

All reagents and solvents were purchased from commercial suppliers and used without further purification unless otherwise stated, except THF, (freshly distilled from Na/benzophenone under nitrogen), and Et₃N, DMF and CH₂Cl₂ (freshly distilled from CaH₂ under nitrogen). USP grade castor oil was purchased at a local pharmacy (Life™ Brand) and used as received. For NMR, chemical shifts are reported in parts per million (ppm) on the δ scale and coupling constants, J, are in hertz (Hz). Multiplicities are reported as “s” (singlet), “d” (doublet), “dd” (doublet of doublets), “dt” (doublet of triplets), “ddd” (doublet of doublets of doublets), “t” (triplet), “td” (triplet of doublets), “q” (quartet), “quin” (quintuplet), “sex” (sextet), “m” (multiplet), and further qualified as “app” (apparent) and “br” (broad).

The steps of the general synthesis of lipid conjugates based on hydroxy and carboxy derivatives of castor oil (ricinolein) are provided below in Scheme 1. This is followed by Scheme 1, referred to as general procedures A-E, describing the steps for producing the pro-drugs of Examples 2A to 2V below.

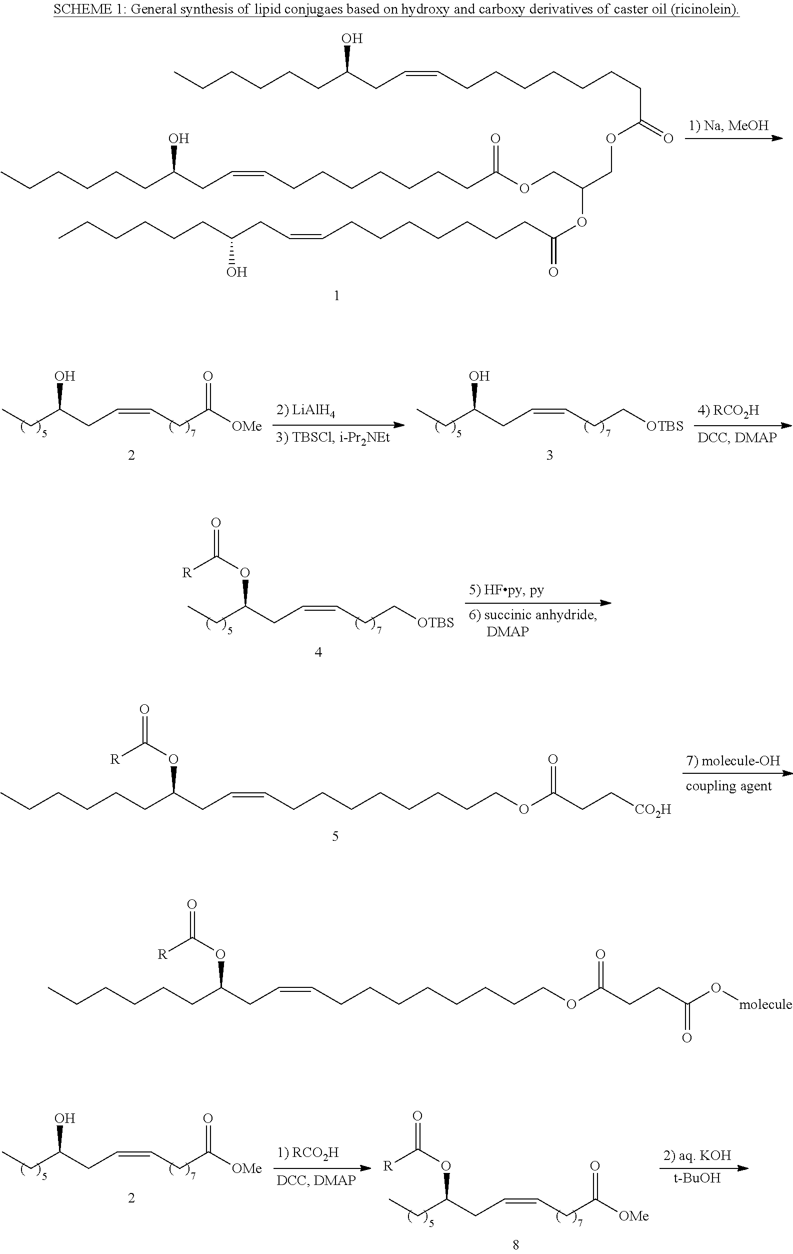

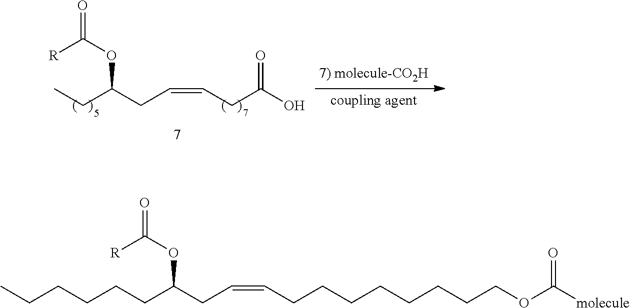

According to the synthesis reaction described above in Scheme I, castor oil, also known as ricinolein (a glyceride of ricnoleic acid) is the starting material for the synthesis of the pro-drugs shown in FIG. 3.

In step 1) above, sodium methoxide (2.0 mL of 3.0 M solution in MeOH, 6.00 mmol, 0.20 equiv.) was added to a stirring, room temperature 1:1 THF/MeOH (30 mL) solution of the castor oil (28.0 g, 30.0 mmol, 1.00 equiv.) in a round bottom flask under argon. After 14 h, the reaction mixture was quenched with saturated aqueous NH₄Cl and extracted with Et₂O (3×150 mL). The combined organic layers were washed with water (1×150 mL), brine (1×150 ml), dried over Na₂SO₄ and concentrated to produce a clear, colourless oil of methyl (12R)-hydroxyoleate 1 (28.0 g, quantitative yield), which was used without further purification. The structure of methyl (12R)-hydroxyoleate and its physical properties are shown below:

### Methyl (12R)-hydroxyoleate (1)

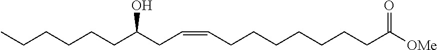 [SMILES: CCCCCC[C@@H](O)C/C=C\CCCCCCCC(=O)OC]

R_{f}=0.50 (SiO₂, 70:30 hexanes/EtOAc);

¹H NMR (300 MHz, CDCl₃): δ 5.64-5.50 (m, 1H), 5.49-5.35 (m, 1H), 3.68 (s, 3H), 3.63 (quint., J=5.6 Hz, 1H), 2.32 (t, J=7.6 Hz, 2H), 2.23 (t, J=6.6 Hz, 2H), 2.13-2.00 (m, 2H), 1.72-1.19 (m, 20H), 0.90 (t, J=6.4 Hz, 3H).

According to 2) in the reaction scheme above, a room temperature THF (15 ml) solution of methyl (12R)-hydroxyoleate (9.37 g, 30.0 mmol) was added from an addition funnel over 20-30 min to a stirred, ice-cold THF (90 mL) suspension of LiAlH₄ (1.25 g, 33.0 mmol, 1.10 equiv.) in a round bottom flask under argon. After the addition was complete, the cold bath was removed. After 14 h, the reaction mixture was cooled in an ice bath, diluted with Et₂O (150 mL) and quenched with a quenching solution (1.25 ml H₂O, 1.25 mL aqueous 1 M NaOH, 3.75 mL H₂O), stirred for 1 h at room temperature and filtered through Celite, while washing thoroughly with Et₂O. The filtrate was concentrated on a rotary evaporator to yield the crude diol as a pale yellow oil (quantitative yield), which was used without further purification.

According to 3) of the above reaction scheme, a room temperature DMF (20 mL) solution of tert-butyldimethylsilyl chloride (3.96 g, 26.2 mmol, 1.00 equiv.) was added from an addition funnel over 30 min to a 10-15° C. DMF (25 ml) solution of the above diol (8.21 g, 28.9 mmol, 1.10 equiv.) and i-Pr₂Net (5.73 mL, 32.8 mmol, 1.25 equiv.) in a round bottom flask under argon. The reaction mixture was allowed to warm up over 14 h, then quenched with saturated aqueous NH₄Cl and extracted with 1:1 Et₂O/hexanes (3×100 ml). The combined organic layers were washed with H₂O (3×100 ml), brine (1×100 ml), dried over Na₂SO₄ and concentrated on a rotary evaporator to produce the crude primary silyl ether as a pale yellow oil. The crude was purified by filtration through a plug of silica gel (220 ml SiO₂, 99:1->95:5 hexanes/EtOAc) to yield a clear, colourless oil composed of the silyl ether 2 (8.38 g, 80% yield). The structure of the silyl ether 2 is shown below, as well as its physical properties:

### I-1-(tert-Butyldimethylsilyl)-12-hydroxyoleyl alcohol (2)

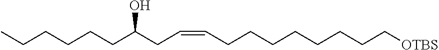 [SMILES: CCCCCC[C@@H](O)C/C=C\CCCCCCCCO[Si](C)(C)C(C)(C)C]

R_{f}=0.16 (SiO₂, 95:5 hexanes/EtOAc);

¹H NMR (300 MHz, CDCl₃): δ 5.64-5.50 (m, 1H), 5.49-5.35 (m, 1H), 3.68 (s, 3H), 3.63 (quint., J=5.6 Hz, 1H), 2.32 (t, J=7.6 Hz, 2H), 2.23 (t, J=6.6 Hz, 2H), 2.13-2.00 (m, 2H), 1.72-1.19 (m, 20H), 0.90 (t, J=6.4 Hz, 3H).

According to 4) of the above reaction scheme, N,N′-Dicyclohexylcarbodiimide (DCC) (495 mg, 2.40 mmol, 1.20 equiv.) was added to an ice-cold CH₂Cl₂ (6 ml) solution of RCO₂H (279 mg, 2.40 mmol, 1.20 equiv.) in a round bottom flask under argon, and the ice bath was subsequently removed and the resultant mixture stirred for 15 min. In this example, RCO₂H was hexanoic acid, although other acyl groups can be utilized to produce a desired hydrocarbon side chain S. The reaction mixture was cooled again in an ice bath, a CH₂Cl₂ (2 mL) solution of the silyl ether, I-1-(tert-Butyldimethylsilyl)-12-hydroxyoleyl alcohol 2 (797 mg, 2.00 mmol) was added, followed by DMAP (366 mg, 3.00 mmol, 1.50 equiv.), and the reaction mixture was allowed to warm to room temperature over 14 h. The reaction mixture was diluted with Et₂O, stirred for 10 min, then filtered through Celite. The filtrate was concentrated on a rotary evaporator to yield the crude ester as a white semi-solid. The crude was purified by filtration through a plug of silica gel (20 mL SiO₂, 95:5 hexanes/EtOAc) to produce a clear, colourless oil as the intermediate ester (quantitative yield) having an R_{f}=0.53 (SiO₂, 90:10 hexanes/EtOAc).

According to 5) of the reaction scheme above, neat HF·pyridine solution (0.74 mL of 70% HF in pyridine, 6.00 mmol, 3.00 equiv.) was added to a stirred, ice-cold THF (6 mL) solution of pyridine (0.48 mL, 6.00 mmol, 3.00 equiv.) and the above silyl ether (2.00 mmol) in a round bottom flask under argon. After 2 h, the reaction mixture was quenched with saturated aqueous NaHCO₃. The mixture was extracted with Et₂O (2×10 ml), then the combined organic extracts were washed with H₂O (1×10 mL), brine, dried over Na₂SO_{A} and concentrated on a rotary evaporator to afford the crude primary alcohol. The crude was purified by filtration through a plug of silica gel (20 mL, 90:10 hexanes/EtOAc) to produce a primary alcohol 3 (quantitative yield) as a clear, colourless oil having the structure and physical properties below:

### (12R)-Hexanoyloxyoleyl alcohol (3)

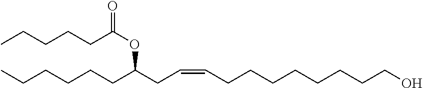 [SMILES: CCCCCC[C@H](C/C=C\CCCCCCCCO)OC(=O)CCCCC]

According to 6) of the reaction scheme above, solid succinic anhydride (400 mg, 4.00 mmol, 2.00 equiv.) and DMAP (611 mg, 5.00 mmol, 2.50 equiv.) were added to a stirring room temperature CH₂Cl₂ (6 ml) solution of the (12R)-Hexanoyloxyoleyl alcohol (3) (765 mg, 2.00 mmol, 1.00 equiv.) in a round bottom flask under argon. After 14 hours, the reaction was quenched with aqueous 1 M HCl and extracted with CH₂Cl₂ (2×15 mL). The combined organic extracts were then washed with aqueous 1 M HCl (1×15 mL), H₂O (2×15 mL), dried over Na₂SO₄ and concentrated on a rotary evaporator to afford the intermediate hemisuccinate (quantitative yield) as a pale yellow oil that was used without further purification. The intermediate had an R_{f}=0.32 (SiO₂, 50:50 hexanes/EtOAc).

According to 7) in the reaction scheme, solid DCC (99 mg, 0.48 mmol, 1.20 equiv.) was added to a stirring, ice-cold CH₂Cl₂ (2 mL) solution of the above hemisuccinate (232 mg, 0.48 mmol, 1.20 equiv.) in a round bottom flask under argon, then the ice bath was removed and the resultant mixture stirred for 15 min. The reaction mixture was cooled again in an ice bath and solid dexamethasone (157 mg, 0.40 mmol) and DMAP (73 mg, 0.60 mmol, 1.50 equiv.) were added. The reaction mixture was allowed to warm up over 14 h, diluted with Et₂O, stirred for 10 min, then filtered through Celite. The filtrate was concentrated to produce the crude, which was a pale yellow oil. The crude was purified by flash column chromatography (50 mL SiO₂, 80:20→50:50 hexanes/EtOAc) to yield a clear, colourless oil as desired pro-drug 4 (328 mg, 95% yield) having the structure and properties below:

### 2-((8S,9R,10S,11S,13S,14S,16R,17R)-9-Fluoro-11,17-dihydroxy-10,13,16-trimethyl-3-oxo-6,7,8,9,10,11,12,13,14,15,16,17-dodecahydro-3H-cyclopenta[a]57ctadic57rene-17-yl)-2-oxoethyl ((R,Z)-12-(hexanoyloxy)57ctadic-9-en-1-yl) succinate (4)

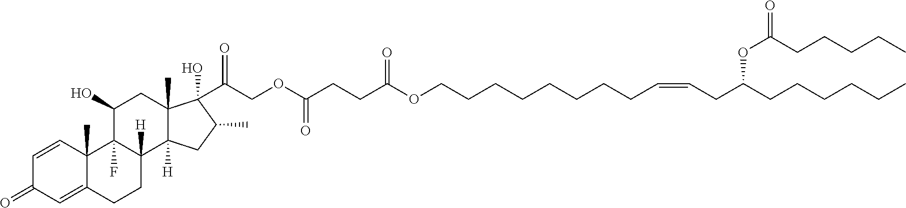

R_{f}=0.38 (SiO₂, 50:50 hexanes/EtOAc);

¹H NMR (300 MHz, CDCl₃): δ 7.22 (dd, J=10.2, 3.9, 1H), 6.32 (dd, J=10.2, 1.7, 1H), 6.1 (s, 1H), 5.44-5.17 (m, 9H), 5.00-4.81 (m, 2H), 4.43-4.22 (m, 4H), 4.21-4.06 (m, 2H), 3.16-3.01 (m, 1H), 2.84-2.51 (m, 11H), 2.50-2.23 (m, 9H), 2.21-1.48 (m, 25H), 1.45-1.15 (m, 34H), 1.14-1.00 (m, 1H), 1.03 (s, 3H), 0.95-0.81 (m, 10H).

The pro-drug is based on a ricinoleyl scaffold L with a hexanoyl (C6:0) side chain conjugated to dexamethasone by a succinate linker (INT-D034).

In the above example, RCO₂H added in 4) of the above reaction was hexanoic acid to produce the hexanoyl side chain (C6:0), although other fatty acids can be utilized to produce a desired hydrocarbon side chain R on the ricinoleyl scaffold.

### General Procedure A—Acylation of (R)-1-(tert-Butyldimethylsilyl)-12-hydroxyoleyl alcohol 3 (4a-h)

DCC (1.20 equiv.) was added to a stirring, ice-cold CH₂Cl₂ solution of the desired carboxylic acid (1.20 equiv.) in a round bottom flask under argon, then the ice bath was removed and the resultant stirred for 15 min. The reaction mixture was cooled again in an ice bath, a CH₂Cl₂ solution of alcohol 3 (1.00 equiv., 0.25 M in CH₂Cl₂) was added, followed by DMAP (1.50 equiv.), and the reaction mixture was allowed to warm to room temperature over 14 h. The reaction mixture was diluted with Et₂O, stirred for 10 min, then filtered through Celite®. The filtrate was concentrated on a rotary evaporator to yield the crude ester as a white semi-solid. The crude was purified by filtration through a plug of silica gel (95:5 hexanes/EtOAc) to afford the pure ester.

General Procedure B—Desilylation-Succinylation of (12R)-Acyloxyoleyl alcohols 4-h (5a-h):

HF·pyridine solution (3.00 equiv. of 70% HF in pyridine) was added to a stirring, ice-cold THF (0.30 M relative to starting silyl ether) solution of pyridine (3.00 equiv.) and 12-acyl ricinoleyl alcohol silyl ether (1.00 equiv.) in a round bottom flask under argon. When TLC indicated consumption of the starting material (2-8 h), the reaction mixture was quenched with saturated aqueous NaHCO₃, The mixture was extracted with Et₂O (2×40 mL), then the combined organic extracts were washed with H₂O (1×10 ml), brine, dried over Na₂SO₄ and concentrated on a rotary evaporator to afford the crude primary alcohol. The crude was purified by filtration through a plug of silica gel (90:10 hexanes/EtOAc), concentrated on a rotary evaporator and dried under high vacuum to afford the primary alcohol as a clear, colourless oil and used in the subsequent succinylation without further purification.

Solid succinic anhydride (2.00 equiv.) and DMAP (2.50 equiv.) were added to a stirring, room temperature CH₂Cl₂ (0.30 M relative to starting primary alcohol) solution of 12-acyl ricinoleyl alcohol (1.00 equiv.) in a round bottom flask under argon. After 14 hours, the reaction was quenched with aqueous 1 M HCl and extracted with CH₂Cl₂ (2×15 mL). The combined organic extracts were then washed with aqueous 1 M HCl (1×45 mL), H₂O (2×15 ml), dried over Na₂SO₄ and concentrated on a rotary evaporator. The residue was redissolved in hexanes, treated with activated carbon, filtered through Celite® and the filtrate concentrated to afford the intermediate hemisuccinate as a colourless to pale yellow oil that was used without further purification.

### General Procedure C—Acylation of Methyl (12R)-Ricinoleate Z (6a-c)

DCC (1.20 equiv.) was added to a stirring, ice-cold CH₂Cl₂ solution of the desired carboxylic acid (1.20 equiv.) in a round bottom flask under argon, then the ice bath was removed and the resultant stirred for 15 min. The reaction mixture was cooled again in an ice bath, a CH₂Cl solution of methyl (12R)-ricinoleate (1.00 equiv., 0.30 M in CH₂Cl₂) was added, followed by DMAP (1.50 equiv.), and the reaction mixture was allowed to warm to room temperature over 14 h. The reaction mixture was diluted with hexanes, stirred for 10 min, then filtered through Celite®. The filtrate was concentrated on a rotary evaporator to yield the crude diester as a white semi-solid, which was purified by filtration through a plug of silica gel (95:5 hexanes/EtOAc) to afford the pure ester.

General Procedure D—Conjugation of Dexamethasone to Hemisuccinates 5a-h:

DCC (1.20 equiv.) was added to a stirring, ice-cold CH₂Cl₂ (0.2 M in dexamethasone) solution of 12-acyl ricinoleyl hemisuccinate (1.20 equiv.) in a round bottom flask under argon, then the ice bath was removed and the resultant stirred for 15 min. The reaction mixture was cooled again in an ice bath and solid dexamethasone (1.00 equiv.) and DMAP (1.50 equiv.) were added. The reaction mixture was allowed to warm up over 14 h, diluted with Et₂O, stirred for 10 min, then filtered through Celite®. The filtrate was concentrated to afford the crude as a pale yellow oil and subsequently purified by flash column chromatography (SiO₂, 80:20-450:50 hexanes/EtOAc) to afford a clear, colourless oil as the desired dexamethasone conjugate.

### General Procedure E—Conjugation of Dexamethasone to Ricnoleic Acids 12a-b, 13

DCC (1.10 equiv.) was added to a stirring, ice-cold CH₂Cl₂ (0.1 M in dexamethasone) solution of the acyloxystearic acid (1.10 equiv.) in a round bottom flask under argon, then the ice bath was removed and the resultant stirred for 15 min. The reaction mixture was cooled again in an ice bath and solid dexamethasone (1.00 equiv.) and DMAP (1.50 equiv.) were added. The reaction mixture was allowed to warm up over 14 h, diluted with Et₂O, stirred for 10 min, then filtered through Celite®. The filtrate was concentrated to afford the crude as a pale yellow oil and subsequently purified by flash column chromatography to afford a clear, colourless oil as the desired conjugate.

### (R,Z)-18-((tert-Butyldimethylsilyl)oxy)octadec-9-en-7-yl acetate (4a)

Acetyl chloride (0.43 mL, 6.00 mmol, 1.20 equiv.) was added dropwise to a stirring ice-cold CH₂Cl₂ (10 mL) solution of silyl ether 3 (2.00 g, 5.00 mmol, 1.00 equiv.), acetyl chloride (0.43 mL, 6.00 mmol, 1.20 equiv.), triethylamine (0.83 mL, 6.00 mmol, 1.2 equiv.) and DMAP (733 mg, 6.00 mmol, 1.20 equiv.) in a round bottom flask under argon, which was allowed to warm to room temperature. After 14 h, the reaction mixture was diluted with CH₂Cl₂, washed with saturated aqueous NH₄Cl (1×15 ml), water (2×15 mL) and dried over Na₂SO₄ and concentrated on a rotary evaporator. The residue was redissolved in eluent and passed through a plug of silica gel (30 mL SiO₂, 97:3 hexanes/EtOAc) to afford ester 4a (1.83 g, 83%) as a pale yellow oil.

R_{f}=0.45 (SiO₂, 95:5 hexanes/EtOAc);

¹H NMR (300 MHz, CDCl₃): δ 5.65-5.53 (m, 1H), 5.49-5.36 (m, 1H), 3.70-3.56 (m, 3H), 2.23 (t, J=6.8 Hz, 2H), 2.13-2.00 (m, 2H), 1.58-1.23 (m, 22H), 0.97-0.86 (m, 12H), 0.07 (s, 6H).

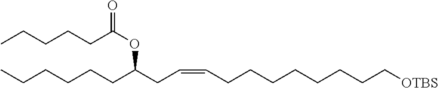 [SMILES: CCCCCC[C@H](C/C=C\CCCCCCCCO[Si](C)(C)C(C)(C)C)OC(=O)CCCCC]

### (R,Z)-18-((tert-Butyidimethylsilyl)oxy)octadec-9-en-7-yl hexanoate (4b)

According to General Procedure A, silyl ether 3 (2.00 g, 5.00 mmol), hexanoic acid (697 mg, 6.00 mmol), DCC (1.24 g, 6.00 mmol) and DMAP (916 mg, 7.50 mmol) in CH₂Cl₂ (15 ml) provided 2.37 g of ester 4b (2.39 g, quantitative yield) as a clear, colourless oil.

R_{f}=0.43 (SiO₂, 95:5 hexanes/EtOAc);

¹H NMR (300 MHz, CDCl₃): δ 5.56-5.42 (m, 1H), 5.41-5.27 (m, 1H), 4.90 (quint., J=6.3 Hz, 1H), 3.61 (t, J=6.6 Hz, 2H), 2.37-2.22 (m, 4H), 2.10-1.96 (m, 2H), 1.71-1.45 (m, 6H), 1.43-1.19 (m, 22H), 0.91 (br s, 15H), 0.07 (s, 6H).

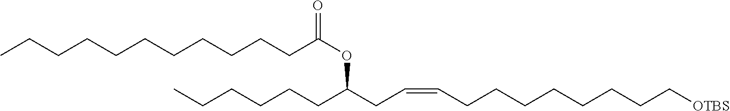 [SMILES: CCCCCCCCCCCC(=O)O[C@@H](C/C=C\CCCCCCCCO[Si](C)(C)C(C)(C)C)CCCCCC]

### (R,Z)-18-((tert-Butyldimethylsilyl)oxy)octadec-9-en-7-yl laurate (4c)

According to General Procedure A, silyl ether 3 (997 mg, 2.50 mmol), lauric acid (601 mg, 3.00 mmol), DCC (619 mg, 3.00 mmol) and DMAP (458 mg, 3.75 mmol) in CH₂Cl₂ (8 mL) provided ester 4c (1.38 g, quantitative yield) as a clear, colourless oil.

R_{f}=0.56 (SiO₂, 95:5 hexanes/EtOAc);

¹H NMR (300 MHz, CDCl₃): δ 5.56-5.41 (m, 1H), 5.41-5.26 (m, 1H), 4.90 (quint., J=6.2 Hz, 1H), 3.61 (t, J=6.6 Hz, 2H), 2.37-2.21 (m, 4H), 2.11-1.95 (m, 2H), 1.72-1.43 (m, 12H), 1.43-1.13 (m, 38H), 0.91 (br s, 15H), 0.07 (s, 6H).

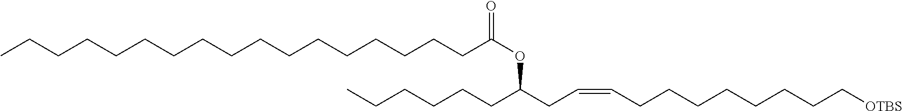 [SMILES: CCCCCCCCCCCCCCCCCC(=O)O[C@@H](C/C=C\CCCCCCCCO[Si](C)(C)C(C)(C)C)CCCCCC]

### (R,Z)-18-((tert-Butyidimethylsilyl)oxy)octadec-9-en-7-yl stearate (4d)

According to General Procedure A, silyl ether 3 (997 mg, 2.50 mmol), stearic acid (853 mg, 3.00 mmol), DCC (619 mg, 3.00 mmol) and DMAP (458 mg, 3.75 mmol) in 2:1 THF/CH₂Cl₂ (6 mL) provided ester 4d (1.56 g, 94%) as a clear, colourless oil.

R_{f}=0.48 (SiO₂, 90:10 hexanes/EtOAc);

¹H NMR (300 MHz, CDCl₃): δ 5.57-5.41 (m, 1H), 5.41-5.25 (m, 1H), 4.90 (quint., J=6.3 Hz, 1H), 3.61 (t, J=6.5 Hz, 2H), 2.39-2.20 (m, 4H), 2.11-1.96 (m, 2H), 1.72-1.43 (m, 8H), 1.43-1.13 (m, 44H), 0.91 (br s, 15H), 0.07 (s, 6H).

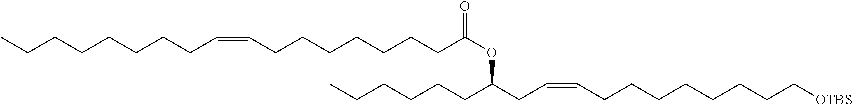 [SMILES: CCCCCCCC/C=C\CCCCCCCC(=O)O[C@@H](C/C=C\CCCCCCCCO[Si](C)(C)C(C)(C)C)CCCCCC]

### (R,Z)-18-((tert-Butyldimethylsilyl)oxy)octadec-9-en-7-yl oleate (4e)

According to General Procedure A, silyl ether 3 (997 mg, 2.50 mmol), oleic acid (847 mg, 3.00 mmol), DCC (619 mg, 3.00 mmol) and DMAP (458 mg, 3.75 mmol) in CH₂Cl₂ (10 mL) provided ester 4e (1.64 g, quantitative) as a clear, colourless oil.

R_{f}=0.41 (SiO₂, 95:5 hexanes/EtOAc);

¹H NMR (300 MHz, CDCl₃): δ 5.56-5.25 (m, 4H), 4.90 (quint., J=6.2 Hz, 1H), 3.61 (t, J=6.5 Hz, 2H), 2.42-2.19 (m, 8H), 2.11-1.93 (m, 6H), 1.70-1.44 (m, 8H), 1.44-1.17 (m, 40H), 0.91 (br s, 15H), 0.06 (s, 6H).

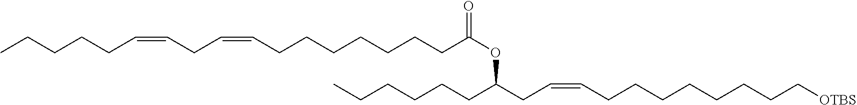 [SMILES: CCCCC/C=C\C/C=C\CCCCCCCC(=O)O[C@@H](C/C=C\CCCCCCCCO[Si](C)(C)C(C)(C)C)CCCCCC]

### (R,Z)-18-((tert-Butyldimethylsilyl)oxy)octadec-9-en-7-yl linoleate (4f)

According to General Procedure A, silyl ether 3 (847 mg, 2.12 mmol), linoleic acid (715 mg, 2.55 mmol), DCC (526 mg, 2.55 mmol) and DMAP (389 mg, 3.19 mmol) in CH₂Cl₂ (7 mL) provided ester 4f (1.06 g, 76%) as a clear, colourless oil.

R_{f}=0.46 (SiO₂, 95:5 hexanes/EtOAc);

¹H NMR (300 MHz, CDCl₃): δ 5.67-5.24 (m, 6H), 4.90 (quint., J=6.2 Hz, 1H), 3.61 (t, J=6.6 Hz, 2H), 2.79 (t, J=5.9 Hz, 2H), 2.40-2.17 (m, 41H), 2.15-1.94 (m, 4H), 1.71-1.44 (m, 8H), 1.43-1.17 (m, 26H), 0.91 (br s, 15H), 0.07 (s, 6H).

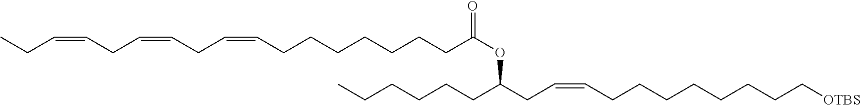 [SMILES: CC/C=C\C/C=C\C/C=C\CCCCCCCC(=O)O[C@@H](C/C=C\CCCCCCCCO[Si](C)(C)C(C)(C)C)CCCCCC]

### (R,Z)-18-((tert-Butyldimethylsilyl)oxy)octadec-9-en-7-yl linolenate (4g)

According to General Procedure A, silyl ether 3 (997 mg, 2.50 mmol), linolenic acid (835 mg, 3.00 mmol), DCC (619 mg, 3.00 mmol) and DMAP (458 mg, 3.75 mmol) in CH₂Cl₂ (8 mL) provided ester 4g (1.52 g, 92%) as a clear, colourless oil.

R_{f}=0.34 (SiO₂, 95:5 hexanes/EtOAc);

¹H NMR (300 MHz, CDCl₃): δ 5.58-5.26 (m, 8H), 4.90 (quint., J=6.2 Hz, 1H), 3.61 (t, J=6.5 Hz, 214), 2.83 (t, J=5.8 Hz, 4H), 2.35-2.22 (m, 4H), 2.17-1.97 (m, 6H), 1.69-1.44 (m, 6H), 1.43-1.18 (m, 26H), 1.00 (t, J=7.5 Hz, 3H), 0.91 (brs, 12H), 0.07 (s, 61H).

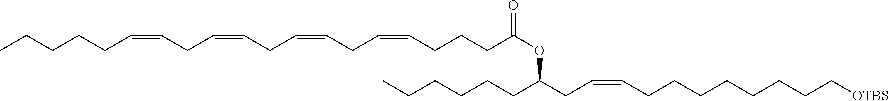 [SMILES: CCCCC/C=C\C/C=C\C/C=C\C/C=C\CCCC(=O)O[C@@H](C/C=C\CCCCCCCCO[Si](C)(C)C(C)(C)C)CCCCCC]

### (R,Z)-18-((tert-Butyldimethylsilyl)oxy)octadec-9-en-7-yl arachidonate (4h)

According to General Procedure A, silyl ether 3 (797 mg, 2.00 mmol), arachidonic acid (670 mg, 2.20 mmol), DCC (227 mg, 2.20 mmol) and DMAP (366 mg, 3.00 mmol) in CH₂Cl₂ (7 mL) provided ester 4h (730 mg, 53%) as a clear, colourless oil after flash column chromatography (99:1495:5 hexanes/EtOAc).

R_{f}=0.57 (SiO₂, 95:5 hexanes/EtOAc);

¹H NMR (300 MHz, CDCl₃): δ 5.57-5.26 (m, 10H), 4.91 (quint., J=6.3 Hz, 1H), 3.61 (t, J=6.5 Hz, 2H), 2.94-2.84 (m, 611), 2.38-2.22 (m, 4H), 2.20-1.96 (m, 6H), 1.71 (quint., J=7.4 Hz, 2H), 1.63-1.46 (m, 4H), 1.45-1.16 (m, 26H), 0.91 (br s, 15H), 0.07 (s, 6H).

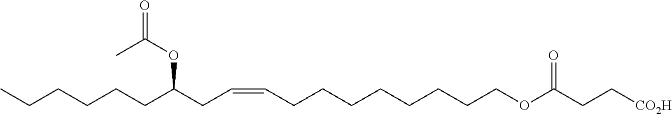 [SMILES: CCCCCC[C@H](C/C=C\CCCCCCCCOC(=O)CCC(=O)O)OC(C)=O]

### (R,Z)-4-((12-Acetoxyoctadec-9-en-1-yl)oxy)-4-oxobutanoic acid (5)

According to General Procedure B, desilylation of silyl ether 4a (1.79 g, 4.07 mmol) with HF·pyridine solution (1.52 mL, 12.2 mmol), pyridine (0.98 mL, 12.2 mmol) and THF (10 mL) gave the intermediate primary alcohol (1.34 g), which was subjected to acylation with succinic anhydride (814 mg, 8.14 mmol), DMAP (1.24 g, 10.2 mmol) and CH₂Cl₂ (10 mL) to afford carboxylic acid 5a (1.72 g, quantitative yield).

R_{f}=0.23 (SiO₂, 50:50 hexanes/EtOAc);

¹H NMR (300 MHz, CDCl₃): δ 5.57-5.42 (m, 1H), 5.42-5.27 (m, 1H), 4.89 (quin., J=6.2 Hz, 1H), 4.11 (t, J=6.7 Hz, 2H), 2.76-2.57 (m, 4H), 2.11-1.97 (m, 2H), 2.05 (s, 3H), 1.72-1.46 (m, 4H), 1.46-1.16 (m, 18H), 0.90 (m, 3H).

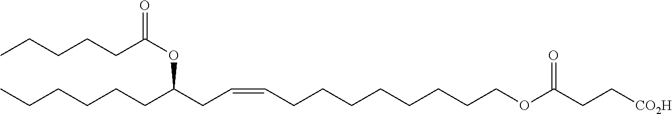 [SMILES: CCCCCC[C@H](C/C=C\CCCCCCCCOC(=O)CCC(=O)O)OC(=O)CCCCC]

### (R,Z)-4-((12-(Hexanoyloxy)octadec-9-en-1-yl)oxy)-4-oxobutanoic acid (5b)

According to General Procedure B, desilylation of silyl ether 4b (2.35 g, 5.00 mmol) with HF·pyridine solution (1.86 mL, 15.0 mmol), pyridine (1.21 mL, 15.0 mmol) and THF (13 mL) gave the intermediate primary alcohol (2.01 g), which was subjected to acylation with succinic anhydride (1.00 g, 10.0 mmol), DMAP (1.53 g, 12.5 mmol) and CH₂Cl₂ (13 mL) to afford carboxylic acid 5b (2.20 g, 92% yield).

R_{f}=0.32 (SiO₂, 50:50 hexanes/EtOAc);

¹H NMR (300 MHz, CDCl₃): δ 5.56-5.42 (m, 1H), 5.41-5.27 (m, 1H), 4.90 (quint., J=6.4 Hz, 1H), 4.11 (t, J=6.5 Hz, 2H), 2.76-2.58 (m, 4H), 2.38-2.22 (m, 4H), 2.11-1.96 (m, 2H), 1.73-1.47 (m, 6H), 1.46-1.15 (m, 22H), 0.97-0.82 (m, 6H).

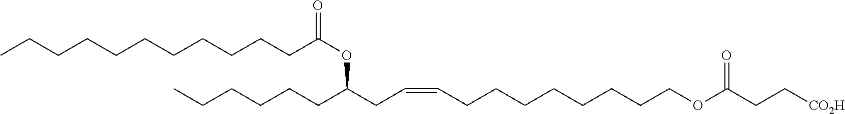 [SMILES: CCCCCCCCCCCC(=O)O[C@@H](C/C=C\CCCCCCCCOC(=O)CCC(=O)O)CCCCCC]

### (R,Z)-4-((12-(Lauroyloxy)octadec-9-en-1-yl)oxy)-4-oxobutanoic acid (5c)

According to General Procedure B, desilylation of silyl ether 4c (1.38 g, 2.50 mmol) with HF·pyridine solution (0.93 mL, 7.50 mmol), pyridine (0.60 ml, 7.50 mmol) and THF (8 mL) gave the intermediate primary alcohol (1.21 g), which was subjected to acylation with succinic anhydride (500 mg, 5.00 mmol), DMAP (764 mg, 6.25 mmol) and CH₂Cl₂ (8 mL) to afford carboxylic acid 5c (1.33 g, 94%).

R_{f}=0.44 (SiO₂, 50:50 hexanes/EtOAc);

¹H NMR (300 MHz, CDCl₃): δ 5.57-5.42 (m, 1H), 5.41-5.26 (m, 111), 4.90 (quint., J=6.2 Hz, 1H), 4.12 (t, J=6.6 Hz, 2H), 2.78-2.59 (m, 4H), 2.37-2.22 (m, 4H), 2.11-1.96 (m, 2H), 1.73-1.45 (m, 611), 1.45-1.12 (m, 28H), 0.98-0.80 (m, 6H).

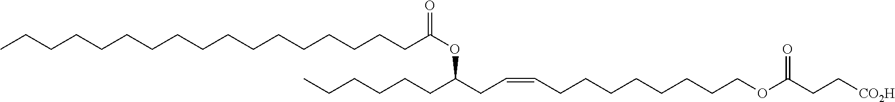 [SMILES: CCCCCCCCCCCCCCCCCC(=O)O[C@@H](C/C=C\CCCCCCCCOC(=O)CCC(=O)O)CCCCCC]

### (R,Z)-4-oxo-4-((12-(Stearoyloxy)octadec-9-en-1-yl)oxy)butanoic acid (5d)

According to General Procedure B, desilylation of silyl ether 4d (1.66 g, 2.50 mmol) with HF·pyridine solution (0.93 mL, 7.50 mmol), pyridine (0.60 ml, 7.50 mmol) and THF (8 mL) gave the intermediate primary alcohol (1.30 g), which was subjected to acylation with succinic anhydride (500 mg, 5.00 mmol), DMAP (764 mg, 6.25 mmol) and CH₂C₂ (8 mL) to afford carboxylic acid 5d (1.29 g, 79% yield).

R_{f}=0.35 (SiO₂, 50:50 hexanes/EtOAc);

¹H NMR (300 MHz, CDCl₃): δ 5.56-5.42 (m, 1H), 5.41-5.27 (m, 1H), 4.90 (quint., J=6.3 Hz, 1H), 4.11 (t, J=6.5 Hz, 2H), 2.77-2.58 (m, 4H), 2.39-2.19 (m, 4H), 2.12-1.95 (m, 2H), 1.73-1.45 (m, 6H), 1.44-1.11 (m, 46H), 0.98-0.80 (m, 6H).

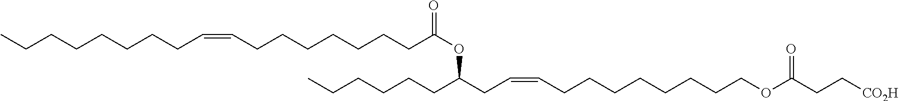 [SMILES: CCCCCCCC/C=C\CCCCCCCC(=O)O[C@@H](C/C=C\CCCCCCCCOC(=O)CCC(=O)O)CCCCCC]

### (R,Z)-4-oxo-4-((12-(Oleoyloxy)octadec-9-en-1-yl)oxy)butanoic acid (5e)

According to General Procedure B, desilylation of silyl ether 4e (663 mg, 1.00 mmol) with HF·pyridine solution (0.37 ml, 3.00 mmol), pyridine (0.24 mL, 3.00 mmol) and THF (5 ml) gave the intermediate primary alcohol (546 mg), which was subjected to acylation with succinic anhydride (200 mg, 2.00 mmol), DMAP (305 mg, 2.50 mmol) and CH₂Cl₂ (5 mL) to afford carboxylic acid 5e (630 mg, 97% yield).

R_{f}=0.42 (SiO₂, 50:50 hexanes/EtOAc);

¹H NMR (300 MHz, CDCl₃): δ 5.57-5.25 (m, 4H), 4.90 (quint., J=6.2 Hz, 1H), 4.11 (t, J=6.5 Hz, 2H), 2.77-2.59 (m, 4H), 2.39-2.20 (m, 4H), 2.13-1.93 (m, 6H), 1.72-1.46 (m, 6H), 1.46-1.02 (m, 34H), 0.97-0.80 (m, 6H),

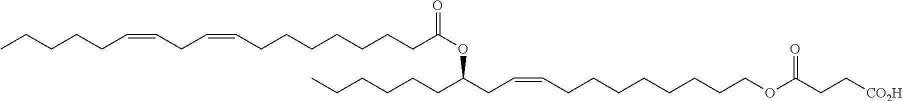 [SMILES: CCCCC/C=C\C/C=C\CCCCCCCC(=O)O[C@@H](C/C=C\CCCCCCCCOC(=O)CCC(=O)O)CCCCCC]

### 4-(((R,Z)-12-(Linoleoyloxy)octadec-9-en-1-yl)oxy)-4-oxobutanoic acid (5f)

According to General Procedure B, desilylation of silyl ether 4f (1.06 g, 1.60 mmol) with HF·pyridine solution (0.60 ml, 4.80 mmol), pyridine (0.39 mL, 4.80 mmol) and THF (8 mL) gave the intermediate primary alcohol (890 mg), which was subjected to acylation with succinic anhydride (320 mg, 3.20 mmol), DMAP (489 mg, 4.00 mmol) and CH₂Cl₂ (8 mL) to afford carboxylic acid 5f (1.04 g, quantitative yield).

R_{f}=0.35 (SiO₂, 50:50 hexanes/EtOAc);

¹H NMR (300 MHz, CDCl₃): δ 5.57-5.26 (m, 6H), 4.90 (quint., J=6.3 Hz, 1H), 4.11 (t, J=6.7 Hz, 2H), 2.79 (t, J=6.0 Hz, 2H), 2.75-2.58 (m, 6H), 2.38-2.20 (m, 4H), 2.14-1.94 (m, 6H), 1.72-1.46 (m, 8H), 1.46-1.14 (m, 30H), 0.98-0.81 (m, 6H).

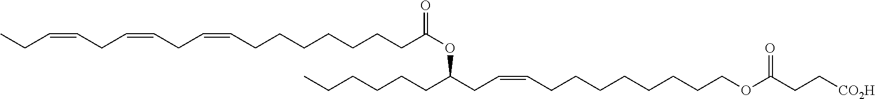 [SMILES: CC/C=C\C/C=C\C/C=C\CCCCCCCC(=O)O[C@@H](C/C=C\CCCCCCCCOC(=O)CCC(=O)O)CCCCCC]

### 4((R,Z)-12-(Linolenoyloxy)octadec-9-en-1-yl)oxy)-4-oxobutanoic acid (5g)

According to General Procedure 8, desilylation of silyl ether 4g (1.54 g, 2.34 mmol) with HF·pyridine solution (0.87 mL, 7.01 mmol), pyridine (0.57 mL, 7.01 mmol) and THF (6 ml) gave the intermediate primary alcohol (1.31 g), which was subjected to acylation with succinic anhydride (468 mg, 4.68 mmol), DMAP (714 mg, 5.84 mmol) and CH₂Cl₂ (6 mL) to afford carboxylic acid 5g (1.47 g, quantitative yield).

R_{f}=0.35 (SiO₂, 50:50 hexanes/EtOAc);

¹H NMR (300 MHz, CDCl₃): δ 5.56-5.25 (m, 8H), 4.90 (quint., J=6.2 Hz, 1H), 4.11 (t, J=6.5 Hz, 2H), 2.82 (t, J=5.7 Hz, 4H), 2.37-2.22 (m, 4H), 2.16-1.95 (m, 6H), 1.74-1.46 (m, 6H), 1.46-1.15 (m, 30H), 0.99 (t, J=7.6 Hz, 3H), 0.94-0.83 (m, 6H).

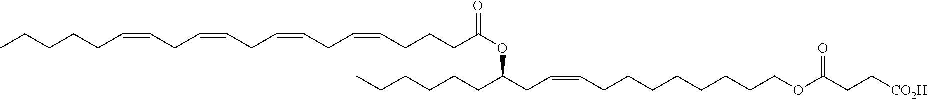 [SMILES: CCCCC/C=C\C/C=C\C/C=C\C/C=C\CCCC(=O)O[C@@H](C/C=C\CCCCCCCCOC(=O)CCC(=O)O)CCCCCC]

### 4-(((R,Z)-12-(Arachidonoyloxy)octadec-9-en-1-yl)oxy)-4-oxobutanoic acid (5h)

According to General Procedure B, desilylation of silyl ether 4h (711 mg, 1.04 mmol) with HF·pyridine solution (0.39 ml, 3.11 mmol), pyridine (0.25 ml, 3.11 mmol) and THF (5 ml) gave the intermediate primary alcohol (593 mg), which was subjected to acylation with succinic anhydride (201 mg, 2.01 mmol), DMAP (306 mg, 2.51 mmol) and CH₂Cl₂ (5 mL) to afford carboxylic acid 5h (582 mg, 87% yield).

R_{f}=0.31 (SiO₂, 50:50 hexanes/EtOAc);

¹H NMR (300 MHz, CDCl₃): δ 5.58-5.24 (m, 10H), 4.90 (quint., J=6.2 Hz, 1H), 4.11 (t, J=6.7 Hz, 2H), 2.93-2.75 (m, 6H), 2.76-2.58 (m, 4H), 2.39-2.22 (m, 41H), 2.20-1.96 (m, 6H), 1.71 (quint., J=7.4 Hz, 2H), 1.69-1.47 (m, 4H), 1.46-1.13 (m, 26H), 0.99-0.80 (m, 6H).

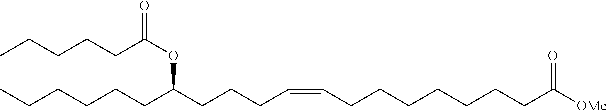 [SMILES: CCCCCC[C@H](CCC/C=C\CCCCCCCC(=O)OC)OC(=O)CCCCC]

### Methyl (12R)-hexanoyloxyoleate (6a)

According to General Procedure C, methyl ricinoleate (2.00 g, 6.40 mmol), hexanoic acid (898 mg, 7.68 mmol), DCC (1.58 g, 7.68 mmol) and DMAP (1.17 g, 9.60 mmol) in CHCl₂ (10 ml) provided, after filtration through silica gel (95:5 hexanes/EtOAc), ricinoleate 6a (2.52 g, 96% yield) as a clear, colourless oil.

R_{f}=0.62 (SiO₂, 70:30 hexanes:EtOAc);

¹H (300 MHz, CDCl₃): δ 5.54-5.42 (m, 1H), 5.40-5.28 (m, 1H), 4.90 (quint., =6.2 Hz, 1H), 3.69 (s, 3H), 2.37-2.23 (m, 6H), 2.11-1.97 (m, 2H), 1.72-1.48 (m, 6), 1.43-1.20 (m, 20), 0.96-0.84 (m, 6H).

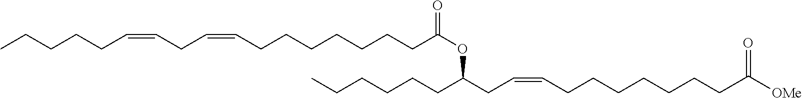 [SMILES: CCCCC/C=C\C/C=C\CCCCCCCC(=O)O[C@@H](C/C=C\CCCCCCCC(=O)OC)CCCCCC]

### Methyl (12R)-linoleoyloxyoleate (6b)

According to General Procedure C, methyl ricinoleate (500 mg, 1.60 mmol), linoleic acid (538 mg, 1.92 mmol), DCC (396 mg, 1.92 mmol) and DMAP (293 mg, 2.40 mmol) in CH₂Cl₂ (5 ml) provided, after filtration through silica gel (95:5 hexanes/EtOAc), ricinoleate 6c (875 g, 93% yield) as a light yellow oil.

R_{f}=0.67 (SiO₂, 80:20 hexanes:EtOAc);

¹H (300 MHz, COCl₃): δ 5.54-5.42 (m, 1H), 5.40-5.28 (m, 1H), 4.90 (quint., J=6.2 Hz, 1H), 3.69 (s, 3H), 2.37-2.23 (m, 6H), 2.11-1.97 (m, 2H), 1.72-1.48 (m, 6), 1.43-1.20 (m, 20), 0.96-0.84 (m, 6H).

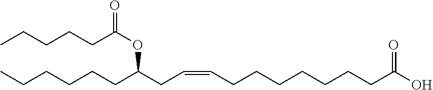 [SMILES: CCCCCC[C@H](C/C=C\CCCCCCCC(=O)O)OC(=O)CCCCC]

### (12R)-Hexanoyloxyoleic acid (7a)

An argon-flushed round bottom flask was charged with methyl ester 6a (1.97 g, 4.79 mmol, 1.00 equiv.) and t-BUOH (12 mL), then aqueous 2.0 M NaOH (1.80 ml, 3.60 mmol, 0.75 equiv.). After 17 h, the pH of the reaction solution was adjusted to 2 using aqueous 1 M HCl and extracted with Et₂O (3×30 ml). The combined organics were washed with water (1×30 ml), brine (1×30 mL), dried over Na₂SO₄ and concentrated on a rotary evaporator under reduced pressure. The residue was filtered through a plug of silica (98:2:0→50:45:5 hexanes:EtOAc:MeOH) to afford carboxylic acid 7a (1.30 g, 92% yield) as a pale yellow oil.

R_{f}=0.24 (SiO₂, 75.20:5 hexanes/EtOAc/MeOH);

¹H NMR (300 MHz, CDCl₃): δ 5.55-5.28 (m, 6H), 4.90 (quint., J=6.2 Hz, 1H), 3.69 (s, 3H), 2.79 (t, J=5.8 Hz, 2H), 2.40-2.21 (m, 6H), 2.16-1.93 (m, 6H), 1.72-1.46 (m, 8H), 1.46-1.18 (m, 32H), 1.00-0.80 (m, 6H).

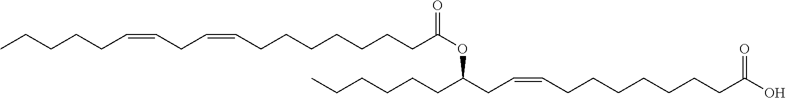 [SMILES: CCCCC/C=C\C/C=C\CCCCCCCC(=O)O[C@@H](C/C=C\CCCCCCCC(=O)O)CCCCCC]

### (12R)-Linoleoyloxyoleic acid (7b)

An argon-flushed round bottom flask was charged with methyl ester 6b (5.97 g, 10.4 mmol, 1.00 equiv.) and t-BuOH (26 mL), then aqueous 2.0 M NaOH (4.70 ml, 9.30 mmol, 0.90 equiv.). After 17 h, the pH of the reaction solution was adjusted to 2 using aqueous 1 M HCl and extracted with Et₂O (3×30 mL). The combined organics were washed with water (1×30 mL), brine (1×30 ml), dried over Na₂SO₄ and concentrated on a rotary evaporator under reduced pressure. The residue was purified by flash column chromatography (SiO₂, 95:5:0→80:15:5 hexanes:EtOAc:MeOH) to afford carboxylic acid 7b (4.48 g, 85% yield) as a pale yellow all.

R_{f}=0.35 (SiO₂, 75:20:5 hexanes/EtOAc/MeOH);

¹H (CDCl₃, 300 MHz): δ 5.55-5.28 (m, 6H), 4.90 (quint., J=6.2 Hz, 1H), 2.79 (t, J=6.0 Hz, 2H), 2.43-2.21 (m, 6H), 2.14-1.96 (m, 6H), 1.73-1.47 (m, 6H), 1.46-1.18 (m, 30H), 0.99-0.81 (m, 6H).

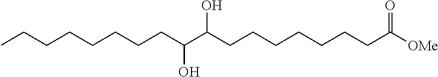 [SMILES: CCCCCCCCC(O)C(O)CCCCCCCC(=O)OC]

### Methyl 9,10-dihydroxystearate (8)

KOH (7.01 g, 125 mmol, 5.00 equiv.) was added to a rapidly stirred room temperature mixture of oleic acid (7.06 g, 25.0 mmol) and water (175 mL) in a 500 ml Erlenmeyer flask, then cooled to ˜10° C. A solution of KMnO₄ (7.11 g, 45.0 mmol, 1.80 equiv.) in water (75 mL) was added dropwise over 10 min. After stirring an additional 10-15 min, the reaction was quenched by addition of saturated aqueous NaHSO₃, then adjusted to pH≤2 by addition of concentrated HCl with the aid of a cooling bath. The white, flocculent mixture was stirred for 1 h at room temperature, then the solids collected by suction filtration and dried in air overnight. The resulting white solids were hot gravity filtered and recrystallized from EtOH to afford the (±)-syn-9,10-dihydroxystearic acid as white crystals (5.86 g, 74% yield).

Concentrated H₂SO₄ (0.06 ml, 1.00 mmol, 0.05 equiv.) was added to a MeOH (50 mL) suspension of the above dihydroxy acid (6.33 g, 20.0 mmol) and the resulting mixture was heated at reflux. After 14 h, the mixture was cooled to room temperature and concentrated on a rotary evaporator under reduced pressure and the resulting residue was partitioned between EtOAc and saturated aqueous NaHCO₃. The organic layer was washed with water (1×75 ml), brine, dried over Na₂SO₄ and concentrated on a rotary evaporator under reduced pressure to afford methyl ester 8 (6.44 g, 97% yield) as a white solid.

R_{f}=0.45 (SiO₂, 50:50 hexanes/EtOAc);

¹H NMR (300 MHz, CDCl₃): δ 3.68 (s, 3H), 3.61 (app br s, 2H), 2.32 (t, J=7.4 Hz, 2H), 2.06-1.95 (app br s, 2H), 1.73-1.16 (m, 26H), 0.96-0.81 (m, 3H).

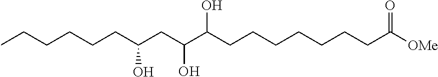 [SMILES: CCCCCC[C@@H](O)CC(O)C(O)CCCCCCCC(=O)OC]

### Methyl 9,10,12R-trihydroxystearate (9)

KOH (5.61 g, 100 mmol, 2.00 equiv.) was added to a rapidly stirred room temperature mixture of ricinoleic acid (14.9 g, 50.0 mmol) and water (500 mL) in a 1 L Erlenmeyer flask, then cooled to ˜10 RC. A solution of KMnO₄ (13.4 g, 85.0 mmol, 1.70 equiv.) in water (250 ml) was added dropwise over 15 min. After stirring an additional 10-15 min, the reaction was quenched by addition of saturated aqueous Na₂SO₃, then adjusted to pH 52 by addition of concentrated HCl with the aid of a cooling bath. The white, flocculent mixture was stirred for 4 h at room temperature, then the solids collected by suction filtration and dried in air overnight. The resulting white solids were hot gravity filtered with EtOH to afford the crude 9,10,12-trihydroxystearic acid, which was used without further purification.

Concentrated H₂SO₄ (0.13 ml, 2.50 mmol, 0.05 equiv.) was added to a MeOH (120 mL) suspension of the above dihydroxy acid (6.33 g, 20.0 mmol) and the resulting mixture was heated at reflux. After 14 h, the mixture was cooled to room temperature and concentrated on a rotary evaporator under reduced pressure and the resulting residue was partitioned between warm EtOAc and saturated aqueous NaHCO₃.

The organic layer was washed with water (1×75 ml), brine, dried over Na₂SO₄ and concentrated on a rotary evaporator under reduced pressure. The resulting pale yellow solid was triturated four times with warm Et₂O to afford methyl ester 9 (9.52 g, 55% yield) as a white solid.

R_{f}=0.33 (SiO₂, 50:50 hexanes/EtOAc);

¹H NMR (300 MHz, CDCl₃): δ 4.07-3.58 (m, 3H), 3.68 (s, 3H), 2.31 (t, J=7.5 Hz, 2H), 1.86-1.14 (m, 24H), 0.90 (br t, 3H).

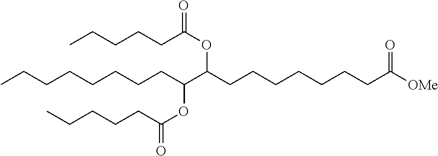 [SMILES: CCCCCCCCC(OC(=O)CCCCC)C(CCCCCCCC(=O)OC)OC(=O)CCCCC]

### Methyl 9,10-dihexanoyloxystearate (10a)

DCC (2.27 g, 11.0 mmol, 2.20 equiv.) was added to a stirring, ice-cold CH₂Cl₂ (13 mL) solution hexanoic acid (1.28 g, 11.0 mmol, 2.20 equiv.) in a round bottom flask under argon, then the ice bath was removed and the resultant stirred for 15 min. The reaction mixture was cooled again in an ice bath, diol 8 (1.65 g, 5.00 mmol) was added, followed by DMAP (1.53 g, 12.5 mmol, 2.50 equiv.), and the reaction mixture was allowed to warm to room temperature over 14 h. The reaction mixture was diluted with Et₂O, stirred for 10 min, then filtered through Celite®. The filtrate was washed with aqueous 1 M HCl (2×30 ml), aqueous 1 M NaOH (2×30 mL), H₂O (1×30 mL), brine, dried over Na₂SO₄ and concentrated on a rotary evaporator under reduced pressure to afford triester 10a (2.61 g, quantitative yield) as a clear, colourless oil.

R_{f}=0.66 (SiO₂, 70:30 hexanes/EtOAc);

¹H NMR (300 MHz, CDCl₃): δ 5.08-4.92 (m, 2H), 3.68 (s, 3H), 2.40-2.20 (m, 6H), 1.74-1.44 (m, 12H), 1.44-1.13 (m, 28H), 1.01-0.80 (m, 9H).

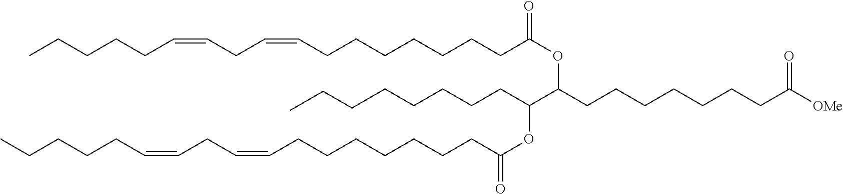 [SMILES: CCCCC/C=C\C/C=C\CCCCCCCC(=O)OC(CCCCCCCC)C(CCCCCCCC(=O)OC)OC(=O)CCCCCCC/C=C\C/C=C\CCCCC]

### Methyl 9,10-dilinoleoyloxystearate (10b)

DCC (4.33 g, 21.0 mmol, 2.10 equiv.) was added to a stirring, ice-cold CH₂Cl₂ (25 mL) solution linoleic acid (5.89 g, 21.0 mmol, 2.20 equiv.) in a round bottom flask under argon, then the ice bath was removed and the resultant stirred for 15 min. The reaction mixture was cooled again in an ice bath, diol 8 (3.30 g, 10.0 mmol) was added, followed by DMAP (3.05 g, 25.0 mmol, 2.50 equiv.), and the reaction mixture was allowed to warm to room temperature over 14 h. The reaction mixture was diluted with hexanes, stirred for 10 min, then filtered through Celite®. The filtrate was concentrated on a rotary evaporator to yield the crude as a white semi-solid, which was purified by filtration through a plug of silica gel (95:5 hexanes/EtOAc) to afford the triester 10b (7.24 g, 85% yield) as a clear colourless oil.

R_{f}=0.57 (SiO₂, 70:30 hexanes/EtOAc);

¹H NMR (300 MHz, CDCl₃): δ 5.49-5.27 (m, 8H), 5.05-4.94 (m, 2H), 3.68 (s, 3H), 2.79 (t, J=5.9 Hz, 4H), 2.39-2.23 (m, 6H), 2.15-1.97 (m, 8H), 1.72-1.45 (m, 10H), 1.45-1.15 (m, 50H), 0.98-0.82 (m, 9H).

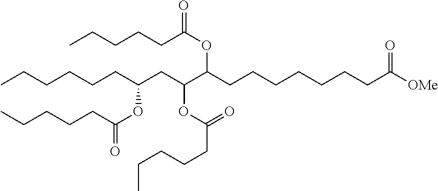 [SMILES: CCCCCC[C@H](CC(OC(=O)CCCCC)C(CCCCCCCC(=O)OC)OC(=O)CCCCC)OC(=O)CCCCC]

### Methyl 9,10,12R-trihexanoyloxystearate (11)

DCC (2.64 g, 12.8 mmol, 3.20 equiv.) was added to a stirring, ice-cold CH₂Cl₂ (13 ml) solution hexanoic add (1.49 g, 12.8 mmol, 3.20 equiv.) in a round bottom flask under argon, then the ice bath was removed and the resultant stirred for 15 min. The reaction mixture was cooled again in an ice bath, triol 9 (1.39 g, 4.00 mmol) was added, followed by DMAP (1.71 g, 14.0 mmol, 3.50 equiv.), and the reaction mixture was allowed to warm to room temperature over 14 h. The reaction mixture was diluted with hexanes, stirred for 10 min, then filtered through Celite®. The filtrate was washed with aqueous 1 M HCl (2×30 ml), aqueous 1 M NaOH (2×30 ml), H₂O (1×30 ml), brine, dried over Na₂SO₄ and concentrated on a rotary evaporator under reduced pressure to afford triester 11 (1.99 g, 78% yield) as a clear, colourless oil.

R_{f}=0.77 (SiO₂, 70:30 hexanes/EtOAc);

¹H NMR (300 MHz, CDCl₃): δ 5.13-4.84 (m, 3H), 3.68 (s, 3H), 2.38-2.19 (m, 8H), 1.92-1.69 (m, 2H), 1.69-1.42 (m, 12H), 1.42-1.16 (m, 28H), 1.00-0.82 (m, 12H).

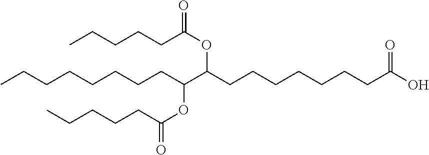 [SMILES: CCCCCCCCC(OC(=O)CCCCC)C(CCCCCCCC(=O)O)OC(=O)CCCCC]

### 9,10-Dihexanoyloxystearic acid (12a)

Aqueous 2.0 M KOH (0.91 mL 1.82 mmol, 1.00 equiv.) was added to a room temperature t-BuOH (7 ml) solution of triester 10a (1.05 g, 2.00 mmol, 1.10 equiv.) in a round bottom flask under argon. After stirring for 20 h, the reaction mixture was acidified to pH 52 by addition of aqueous 3 M HCl and extracted with Et₂O (3×20 mL). The combined organic layers were washed with brine, dried over Na₃SO₄ and concentrated on a rotary evaporator under reduced pressure. The crude residue was purified by flash column chromatography (90:5:5→85:10:5 hexanes/EtOAc/MeOH) to afford carboxylic acid 12a (802 mg, 86% yield) as a clear, colourless oil.

R_{f}=0.22 (SiO₂, 85:10:5 hexanes/EtOAc/MeOH);

¹H NMR (300 MHz, CDCl₃): δ 5.08-4.93 (m, 2H), 2.36 (t, J=7.8 Hz, 2H), 2.30 (t, J=7.6 Hz, 4H), 1.72-1.44 (m, 10H), 1.44-1.16 (m, 30H), 0.97-0.83 (m, 9H).

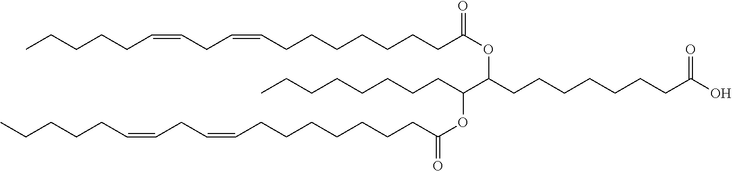 [SMILES: CCCCC/C=C\C/C=C\CCCCCCCC(=O)OC(CCCCCCCC)C(CCCCCCCC(=O)O)OC(=O)CCCCCCC/C=C\C/C=C\CCCCC]

### 9,10-Dilinoleoyloxystearic acid (12b)

Aqueous 2.0 M KOH (3.00 ml, 6.00 mmol, 1.00 equiv.) was added to a room temperature t-BuOH (7 mL) solution of triester 10b (5.64 g, 6.60 mmol, 1.10 equiv.) in a round bottom flask under argon. After stirring for 20 h, the reaction mixture was acidified to pH ≤2 by addition of aqueous 3 M HCl and extracted with hexanes (3×75 ml). The combined organic layers were washed with brine, dried over Na₂SO₄ and concentrated on a rotary evaporator under reduced pressure. The crude residue was purified by flash column chromatography (90:10:0→85:10:5 hexanes/EtOAc/MeOH) to afford carboxylic acid 12b (2.39 g, 68% yield) as a clear, colourless oil.

R_{f}=0.33 (SiO₂, 85:10:5 hexanes/EtOAc/MeOH);

¹H NMR (300 MHz, CDCl₃): δ 5.49-5.25 (m, 8H), 5.07-4.93 (m, 2H), 2.79 (t, J=5.9 Hz, 4H), 2.36 (t, J=7.7 Hz, 2H), 2.30 (t, J=7.5 Hz, 4H), 2.13-2.00 (m, 8H), 1.72-1.45 (m, 10H), 1.45-1.15 (m, 50H), 0.98-0.81 (m, 9H).

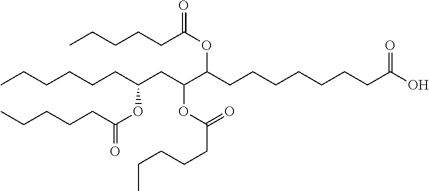 [SMILES: CCCCCC[C@H](CC(OC(=O)CCCCC)C(CCCCCCCC(=O)O)OC(=O)CCCCC)OC(=O)CCCCC]

### 9,10,12R-Trihexanoyloxystearic acid (13)

Aqueous 2.0 M KOH (1.47 mL, 2.94 mmol, 1.00 equiv.) was added to a room temperature t-BuOH (10 mL) solution of tetraester 11 (1.98 g, 3.10 mmol, 1.10 equiv.) in a round bottom flask under argon. After stirring for 20 h, the reaction mixture was acidified to pH 52 by addition of aqueous 3 M HCl and extracted with hexanes (3×30 mL). The combined organic layers were washed with brine, dried over Na₂SO₄ and concentrated on a rotary evaporator under reduced pressure. The crude residue was purified by flash column chromatography (90:10:0→85:10:5→75:20:5 hexanes/EtOAc/MeOH) to afford carboxylic acid 13 (1.40 g, 78% yield) as a clear, colourless oil.

R_{f}=0.32 (SiO₂, 80:15:5 hexanes/EtOAc/MeOH);

¹H NMR (300 MHz, CDCl₃): δ 5.13-4.82 (m, 3H), 2.42-2.18 (m, 8H), 1.92-1.69 (m, 2H), 1.69-1.43 (m, 12H), 1.43-1.14 (m, 28H), 0.99-0.81 (m, 12H).

### Example 2A: Synthesis of INT-D047

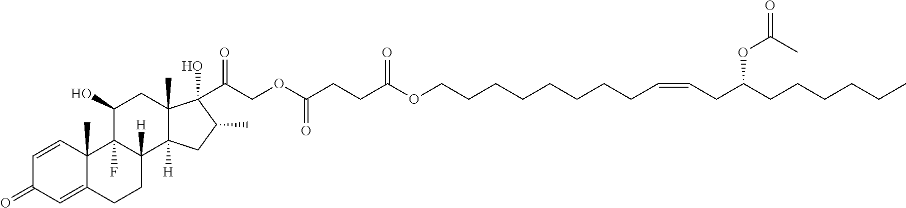

### (R,Z)-12-acetoxyoctadec-9-en-1-yl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) succinate (INT-D047)

According to General Procedure D, dexamethasone (294 mg, 0.75 mmol), hemisuccinate 5a (384 mg, 0.90 mmol), DCC (186 mg, 0.90 mmol), DMAP (137 mg, 1.12 mmol) and CH₂Cl₂ (4 mL) afforded, after flash column chromatography (SiO₂, 80:20→50:50 hexanes/EtOAc), INT-D047 (541 mg, 90% yield) as a clear, colourless oil.

R_{f}=0.36 (SiO₂, 50:50 hexanes/EtOAc);

¹H NMR (300 MHz, CDCl₃): δ 7.21 (d, J=10.1 Hz), 6.36 (dd, J=10.2, 1.7 Hz), 6.13 (s, 1H), 5.58-5.43 (m, 1H), 5.43-5.28 (m, 1H), 4.92 (s, 2H), 4.89 (quint., J=6.4 Hz), 4.45-4.34 (m, 1H), 4.11 (t, J=6.7 Hz, 2H), 3.20-3.04 (m, 1H), 2.86-2.55 (m, 5H), 2.52-2.26 (m, 4H), 2.24-2.12 (m, 1H), 2.11-1.99 (m, 1H), 2.05 (s, 3H), 1.90-1.46 (m, 12H), 1.44-1.17 (m, 15H), 1.06 (s, 3H), 0.99-0.84 (m, 6H).

### Example 2B: Synthesis of INT-D046

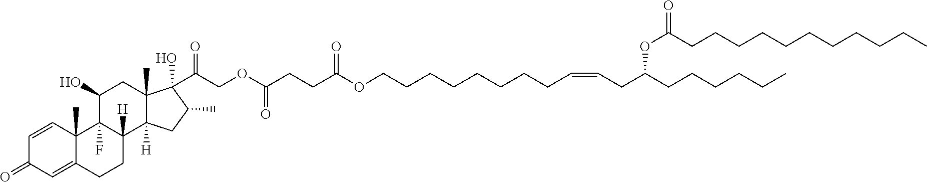

### (R,Z)-12-(dodecanoyloxy)octadec-9-en-1-yl (2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl) succinate (INT-D046)

According to General Procedure D, dexamethasone (157 mg, 0.40 mmol), hemisuccinate 5c (272 mg, 0.48 mmol), DCC (99 mg, 0.48 mmol), DMAP (73 mg, 0.60 mmol) and CH₂Cl₂ (2 mL) afforded, after flash column chromatography (SiO₂, 80:20→50:50 hexanes/EtOAc), INT-D046 (363 mg, 96% yield) as a clear, colourless oil.

R_{f}=0.48 (SiO₂, 50:50 hexanes/EtOAc);

¹H NMR (300 MHz, CDCl₃): δ 7.21 (d, J=10.1 Hz), 6.36 (dd, J=10.2, 1.7 Hz), 6.13 (s, 1H), 5.57-5.42 (m, 1H), 5.40-5.28 (m, 1H), 4.92 (s, 2H), 4.90 (quint., J=6.4 Hz), 4.44-4.33 (m, 1H), 4.11 (t, J=6.9 Hz, 2H), 3.20-3.03 (m, 1H), 2.85-2.54 (m, 5H), 2.53-1.94 (m, 10H), 1.93-1.48 (m, 15H), 1.45-1.14 (m, 28H), 1.06 (s, 3H), 0.98-0.82 (m, 9H).

### Example 2C; Synthesis of INT-D050

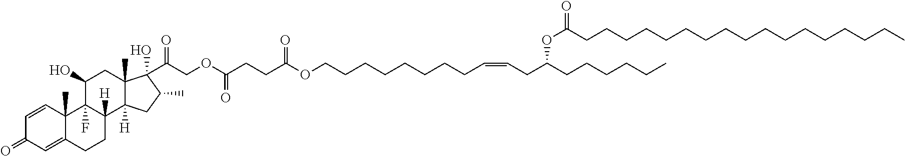

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl ((R,Z)-12-(stearoyloxy)octadec-9-en-1-yl) succinate (INT-D050): JZ-25-009

According to General Procedure D, dexamethasone (392 mg, 1.00 mmol), hemisuccinate 5d (781 mg, 1.28 mmol), DCC (248 mg, 1.28 mmol), DMAP (183 mg, 1.50 mmol) and CH₂Cl₂ (5 mL) afforded, after flash column chromatography (SiO₂, 80:20→50:50 hexanes/EtOAc), INT-DO (933 mg, 91% yield) as a clear, colourless oil.

R_{f}=0.42 (SiO₂, 50:50 hexanes/EtOAc);

¹H NMR (300 MHz, CDCl₃): δ 7.22 (d, J=10.1 Hz), 6.36 (dd, J=10.1, 1.5 Hz), 6.13 (s, 1H), 5.55-5.41 (m, 1H), 5.40-5.27 (m, 1H), 4.93 (s, 2H), 4.89 (quint., J=6.2 Hz), 4.44-4.33 (m, 1H), 4.10 (t, J=6.9 Hz, 2H), 3.20-3.04 (m, 1H), 2.85-2.55 (m, 5H), 2.53-1.95 (m, 11H), 1.91-1.45 (m, 15H), 1.43-1.15 (m, 44H), 1.06 (s, 3H), 0.98-0.81 (m, 9H).

### Example 2D: Synthesis of INT-D035

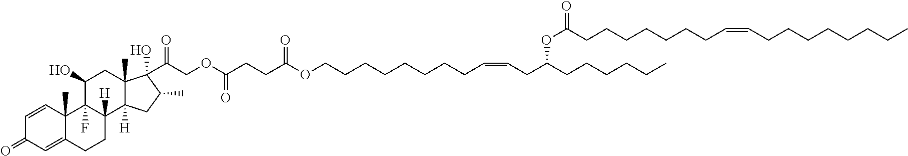

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl ((R,Z)-12-(oleoyloxy)octadec-9-en-1-yl) succinate (INT-DOSS)

According to General Procedure D, dexamethasone (133 mg, 0.34 mmol), hemisuccinate 5e (264 mg, 0.41 mmol), DCC (84 mg, 0.41 mmol), DMAP (62 mg, 0.51 mmol) and CH₂Cl₂ (2 mL) afforded, after flash column chromatography (SiO₂, 80:20→50:50 hexanes/EtOAc), INT-D035 (330 mg, 95% yield) as a clear, colourless oil.

R_{f}=0.49 (SiO₂, 50:50 hexanes/EtOAc);

¹H NMR (300 MHz, CDCl₃): δ 7.22 (d, J=10.1 Hz), 6.36 (dd, J=10.2, 1.8 Hz), 6.13 (s, 1H), 5.55-5.26 (m, 4H), 4.92 (s, 2H), 4.89 (quint., J=6.3 Hz), 4.44-4.34 (m, 1H), 4.11 (t, J=6.8 Hz, 2H), 3.20-3.04 (m, 1H), 2.85-2.54 (m, 5H), 2.54-1.94 (m, 13H), 1.92-1.46 (m, 16H), 1.44-1.16 (m, 36H), 1.06 (s, 3H), 0.99-0.81 (m, 9H).

### Example 2E: Synthesis of INT-D045

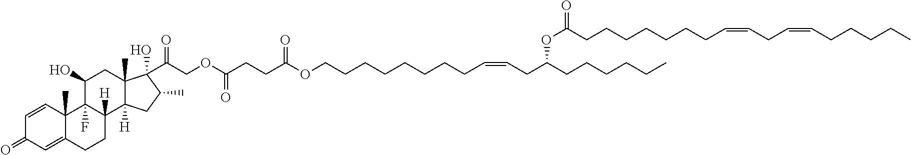

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl ((R,Z)-12-(linoleoyloxy)octadec-9-en-1-yl) succinate (INT-0045)

According to General Procedure D, dexamethasone (157 mg, 0.40 mmol), hemisuccinate 5f (310 mg, 0.48 mmol), DCC (99 mg, 0.48 mmol), DMAP (73 mg, 0.60 mmol) and CH₂Cl₂ (2 mL) afforded, after flash column chromatography (SiO₂, 80:20→50:50 hexanes/EtOAc), INT-D045 (278 mg, 68% yield) as a clear, colourless oil.

R_{f}=0.50 (SiO₂, 50:50 hexanes/EtOAc);

¹H NMR (300 MHz, CDCl₃): δ 7.22 (d, J=10.1 Hz), 6.36 (dd, J=10.2, 1.8 Hz), 6.13 (s, 1H), 5.56-5.25 (m, 6H), 4.93 (s, 2H), 4.89 (quint., J=6.3 Hz), 4.46-4.31 (m, 1H), 4.10 (t, J=6.8 Hz, 2H), 3.20-3.04 (m, 1H), 2.88-2.54 (m, 7H), 2.53-1.91 (m, 15H), 1.90-1.46 (m, 14H), 1.47-1.12 (m, 34H), 1.06 (s, 3H), 0.99-0.81 (m, 9H).

### Example 2F: Synthesis of INT-D049

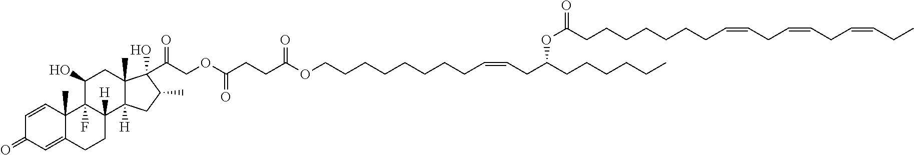

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl ((R,Z)-12-(linolenoyloxy)octadec-9-en-1-yl) succinate (INT-D049)

According to General Procedure D, dexamethasone (294 mg, 0.75 mmol), hemisuccinate 5g (264 mg, 0.90 mmol), DCC (84 mg, 0.90 mmol), DMAP (137 mg, 1.12 mmol) and CH₂Cl₂ (4 mL) afforded, after flash column chromatography (SiO₂, 80:20→50:50 hexanes/EtOAc), INT-D049 (740 mg, 96% yield) as a clear, colourless oil.

R_{f}=0.42 (SiO₂, 50:50 hexanes/EtOAc);

¹H NMR (300 MHz, CDCl₃): δ 7.21 (d, J=10.1 Hz), 6.36 (dd, J=10.2, 1.8 Hz), 6.13 (s, 1H), 5.56-5.25 (m, 8H), 4.93 (s, 2H), 4.89 (quint., J=6.3 Hz), 4.46-4.32 (m, 1H), 4.10 (t, J=6.9 Hz, 2H), 3.22-3.03 (m, 1H), 2.90-2.53 (m, 9H), 2.53-1.91 (m, 17H), 1.90-1.44 (m, 14H), 1.46-1.12 (m, 28H), 1.06 (s, 3H), 0.99 (t, J=7.6 Hz, 3H) 0.96-0.81 (m, 6H).

### Example 26: Synthesis of INT-D051

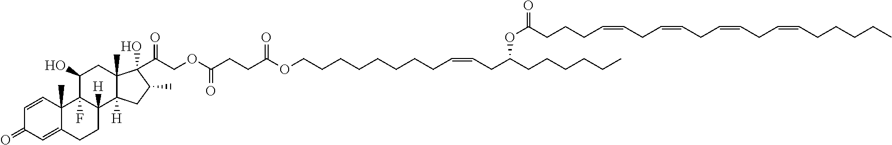

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl ((R,Z)-12-(arachidonoyloxy)octadec-9-en-1-yl) succinate (INT-D051)

According to General Procedure D, dexamethasone (303 mg, 0.77 mmol), hemisuccinate 5f (570 mg, 0.85 mmol), DCC (175 mg, 0.85 mmol), DMAP (142 mg, 1.16 mmol) and CH₂Cl₂ (5 mL) afforded, after flash column chromatography (SO₂, 80:20-450:50 hexanes/EtOAc), INT-D051 (758 mg, 94% yield) as a clear, colourless oil.

R_{f}=0.29 (SiO₂, 50:50 hexanes/EtOAc);

¹H NMR (300 MHz, CDCl₃): δ 7.22 (d, J=10.2 Hz), 6.36 (dd, J=10.2, 1.8 Hz), 6.13 (s, 1H), 5.56-5.26 (m, 10H), 4.93 (s, 2H), 4.88 (quint., J=6.3 Hz), 4.43-4.33 (m, 1H), 4.10 (t, J=6.9 Hz, 2H), 3.20-3.03 (m, 1H), 2.94-2.55 (m, 11H), 2.54-1.95 (m, 17H), 1.91-1.42 (m, 14H), 1.47-1.15 (m, 28H), 1.05 (s, 3H), 1.00-0.81 (m, 9H).

### Example 2H: Synthesis of INT-DOSS

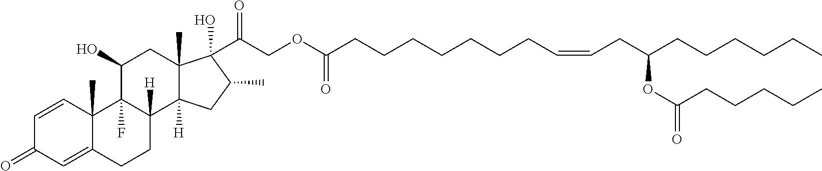

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl (R,Z)-12-hexanoyloxyoleate (INT-D055)

An argon-flushed round bottom flask was charged with carboxylic acid 7a (114 mg, 0.30 mmol, 1.20 equiv.) and CH₂Cl₂ (1.2 mL) and cooled with an ice bath. To this flask, DCC (63 mg, 0.30 mmol, 1.2 equiv.) was added and left to stir for 15 min in ambient room temperature. The flask was again cooled with an ice bath and a mixture of dexamethasone (99 mg, 0.25 mmol, 1.00 equiv.) and DMAP (47 mg, 0.38 mmol, 1.50 equiv.) in CH₂Cl₂ (1.3 mL) prepared in another argon-flushed round bottom flask was added in via syringe. After 17 h, the reaction mixture was diluted with Et₂O, filtered through Celite®, then concentrated on a rotary evaporator under reduced pressure. The crude residue was purified via flash column (80:20→50:50 hexanes/EtOAc) to afford INT-D055 as a pale yellow viscous oil (185 mg, 96% yield).

R_{f}=0.15 (SiO₂, 70:30 hexanes:EtOAc);

¹H (300 MHz, CDCl₃): δ 7.22 (d, J=10.2 Hz, 1H), 6.36 (dd, J=10.2, 1.6 Hz, 1H), 6.14 (s, 1H), 5.55-5.42 (m, 1H), 5.40-5.28 (m, 1H), 4.96-4.82 (m, 3H), 4.44-4.34 (m, 1H), 3.21-3.03 (m, 1H), 2.73-2.55 (m, 1H), 2.53-1.97 (m, 13H), 1.91-1.48 (m, 12H), 1.44-1.18 (m, 21H), 1.07 (s, 3H), 0.98-83 (m, 9H).

### Example 21: Synthesis of INT-D089

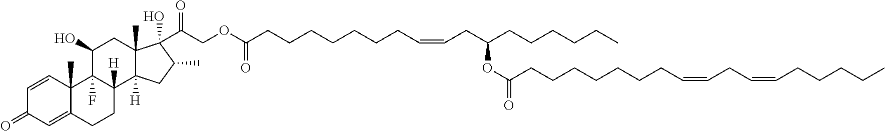

### 2-((8S,9R,10S,11S,13S,14S,16R,17R)-9-Fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl (R,Z)-12-linoleoyloxyoleate (INT-DX9)

An argon-flushed round bottom flask was charged with carboxylic acid 7b (600 mg, 1.07 mmol, 1.20 equiv.) and CH₂C₂ (4.4 mL) and cooled with an ice bath. To this flask, DCC (221 mg, 1.07 mmol, 1.20 equiv.) was added and left to stir for 15 min in ambient room temperature. The flask was again cooled with an ice bath and a mixture of dexamethasone (350 mg, 0.89 mmol, 1.00 equiv.) and DMAP (163 mg, 1.34 mmol, 1.50 equiv.) in CH₂Cl (4.5 mL) prepared in another argon-flushed round bottom flask was added in via syringe. After 17 h, the reaction mixture was diluted with hexanes, filtered through Celite®, then concentrated on a rotary evaporator under reduced pressure. The crude residue was purified via flash column (80:20→50:50 hexanes/EtOAc) to afford INT-D089 as a pale yellow viscous oil (750 mg, 90% yield).

R_{f}=0.46 (silica, 50:50 hexanes:EtOAc);

¹H (300 MHz, CDCl₃): δ 7.22 (d, J=10.3 Hz, 1H), 6.36 (dd, J=10.2, 1.5 Hz, 1H), 6.13 (s, 1H), 5.55-5.28 (m, 6H), 4.97-4.82 (m, 3H), 4.46-4.33 (m, 1H), 3.21-3.04 (m, 1H), 2.79 (t, J=5.9 Hz, 2H), 2.71-2.55 (m, 1H), 2.53-1.97 (m, 16H), 1.91-1.46 (m, 14H), 1.45-1.19 (m, 30H), 1.07 (s, 3H), 0.98-0.84 (m, 9H).

### Example 2J: Synthesis of INT-D085

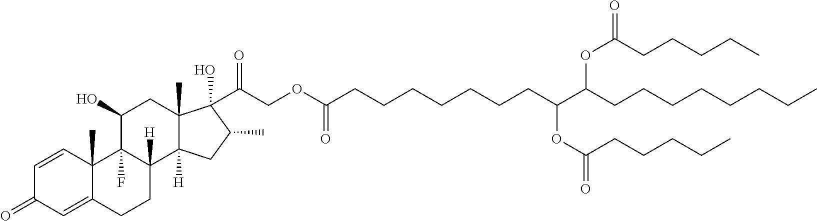

### 1-(2-((8S,9R,10S,11S,13,14S,16R,17R)-9-Fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)-1-oxooctadecane-9,10-diyl dihexanoate (INT-D085)

According to General Procedure E, dexamethasone (235 mg, 0.60 mmol), carboxylic acid 12a (338 mg, 0.66 mmol), DCC (136 mg, 0.66 mmol), DMAP (110 mg, 0.90 mmol) and CH₂Cl₂ (6 mL) afforded, after flash column chromatography (SiO₂, 80:20→50:50 hexanes/EtOAc), INT-D085 (336 mg, 63% yield) as a clear, colourless oil.

R_{f}=0.52 (SiO₂, 50:50 hexanes/EtOAc);

¹H NMR (300 MHz, CDCl₃): δ 7.22 (d, J=10.1 Hz), 6.35 (dd, J=10.2, 1.7 Hz, 1H), 6.12 (s, 1H), 5.07-4.94 (m, 2H), 4.90 (s, 2H), 4.44-4.32 (m, 1H), 3.21-3.02 (m, 1H), 2.63 (dt, J=13.4, 5.4 Hz, 1H), 2.53-2.26 (m, 6H), 2.30 (t, J=7.3 Hz, 4H), 2.24-2.09 (m, 1H), 2.03 (br s, 1H), 1.92-1.44 (m, 18H), 1.43-1.15 (m, 30H), 1.06 (s, 3H), 0.99-0.79 (m, 12H).

### Example 2K: Synthesis of INT-D086

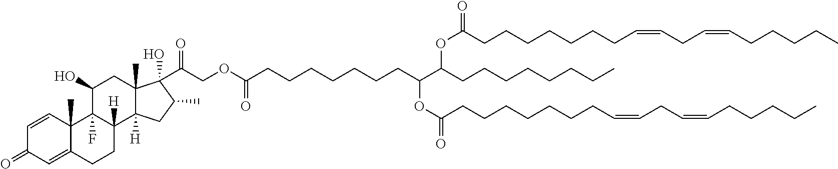

### 1-(2-((8S,9R,10S,11S,13S,14S,16R,17R)-9-Fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)-1-oxooctadecane-9,10-diyl (9Z,9′Z,12Z,12′4)-bis(octadeca-9,12-dienoate) (INT-D86)

According to General Procedure E, dexamethasone (235 mg, 0.60 mmol), carboxylic acid 12b (555 mg, 0.66 mmol), DCC (136 mg, 0.66 mmol), DMAP (110 mg, 0.90 mmol) and CH₂Cl₂ (6 mL) afforded, after flash column chromatography (SiO₂, 80:20→450:50 hexanes/EtOAc), INT-D086 (584 mg, 80% yield) as a clear, colourless oil.

R_{f}=0.28 (SiO₂, 70:30 hexanes/EtOAc);

¹H NMR (300 MHz, CDCl₃): δ 7.22 (d, J=10.1 Hz, 1H), 6.35 (dd, J=10.1, 1.6 Hz, 1H), 6.12 (s, 1H), 5.48-5.24 (m, 8H), 5.06-4.93 (m, 2H), 4.90 (s, 2H), 4.44-4.31 (m, 1H), 3.21-3.02 (m, 1H), 2.78 (t, J=5.9 Hz, 6H), 2.63 (dt, J=13.7, 5.9 Hz, 1H), 2.52-2.33 (m, 6H), 2.30 (t, J=7.4 Hz, 4H), 2.24-1.97 (m, 9H), 1.94-1.45 (m, 18H), 1.44-1.16 (m, 52H), 1.07 (s, 3H), 0.98-0.82 (m, 12H).

### Example 2L: Synthesis of INT-D056

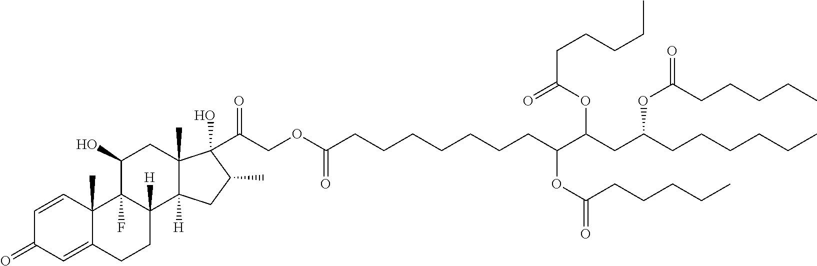

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl 9,10,12R-trihexanoyloxystearate (INT-D056)

According to General Procedure E, dexamethasone (175 mg, 0.44 mmol), carboxylic acid 13 (307 mg, 0.49 mmol), DCC (101 mg, 0.49 mmol), DMAP (82 mg, 0.67 mmol) in CH₂Cl₂ (5 mL) provided, after flash column chromatography (SiO₂, 80:20→50:50 hexanes/EtOAc), INT-D056 as a clear, colourless oil (318 mg, 90% yield).

R_{f}=0.50 (SO₂, 50:50 hexanes/EtOAc);

¹H NMR (300 MHz, CDCl₃): δ 7.23 (d, J=10.2 Hz, 1H), 6.33 (dd, J=10.1, 1.7 Hz, 1H), 6.10 (s, 1H), 5.11-4.89 (m, 3H), 4.90 (s, 21H), 4.42-4.29 (m, 1H), 3.20-3.00 (m, 1H), 2.61 (dt, J=13.5, 5.4 Hz, 1H), 2.52-2.05 (m, 12H), 1.93-1.42 (m, 20H), 1.42-1.14 (m, 28H), 1.04 (s, 3H), 0.98-0.79 (m, 15H).

### Example 2M: Synthesis of INT-D059

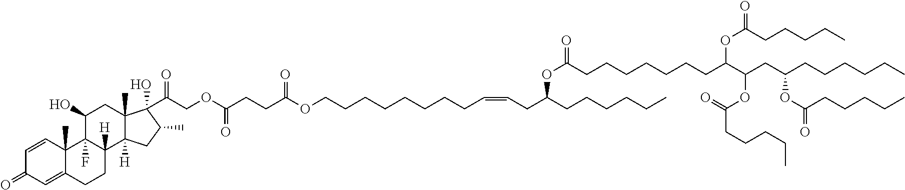

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl ((12S,Z)-12-(((12S)-9,10,12-tris(hexanoyloxy)octadecanoyl)oxy)octadec-9-en-1-yl) succinate (INT-D059)

According to General Procedure E, dexamethasone (137 mg, 0.35 mmol), hemisuccinate derived from ricinoleyl alcohol 2 and carboxylic acid 13 (382 mg, 0.38 mmol), DCC (79 mg, 0.38 mmol), DMAP (64 mg, 0.52 mmol) and CH₂Cl₂ (3.5 mL) afforded, after flash column chromatography (SiO₂, 70:30-450:50 hexanes/EtOAc), INT-D059 (336 mg, 66% yield) as a clear, colourless oil.

R_{f}=0.50 (SiO₂, 50:50 hexanes/EtOAc);

¹H NMR (300 MHz, CDCl₃): δ 7.22 (d, J=10.1 Hz, 1H), 6.36 (dd, J=10.2, 1.7 Hz, 1H), 6.13 (s, 1H), 5.55-5.41 (m, 1H), 5.40-5.27 (m, 1H), 5.12-4.83 (m, 5H), 4.93 (s, 2H), 4.44-4.33 (m, 1H), 4.10 (t, J=6.8 Hz, 2H), 3.20-3.04 (m, 1H), 2.84-2.54 (6H), 2.52-2.10 (m, 18H), 2.10-1.97 (m, 2H), 1.93-1.43 (m, 32H), 1.43-1.16 (m, 62H), 1.05 (s, 3H), 0.99-0.81 (m, 21H).

### Example 2N: Synthesis of INT-D060

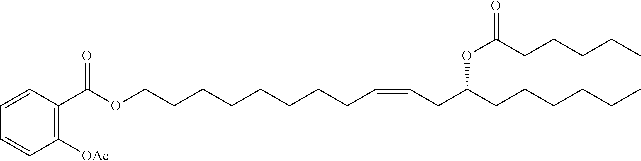 [SMILES: CCCCCC[C@H](C/C=C\CCCCCCCCOC(=O)c1ccccc1OC(C)=O)OC(=O)CCCCC]

### (R,Z)-12-Hexanoyloxyoctadec-9-en-1-yl 2-acetoxybenzoate (INT-D060)

HF·pyridine solution (0.53 mL of 70% HF in pyridine, 4.20 mmol, 3.00 equiv.) was added to a stirring, ice-cold THF (7 mL) solution of pyridine (0.34 mL, 4.20 mmol, 3.00 equiv.) and silyl ether 4b (700 mg, 1.41 mmol) in a round bottom flask under argon. When TLC indicated consumption of the starting material (2-8 h), the reaction mixture was quenched with saturated aqueous NaHCO₃. The mixture was extracted with Et₂O (2×10 ml), then the combined organic extracts were washed with H₂O (140 ml), brine, dried over Na₂SO₄ and concentrated on a rotary evaporator to afford the crude primary alcohol. The crude was purified by filtration through a plug of silica gel (90:10 hexanes/EtOAc) and concentrated on a rotary evaporator to afford the intermediate primary alcohol (518 mg) as a clear, colourless oil that was used without further purification.

A flame-dried and argon-flushed round bottom flask was charged with the above alcohol, pyridine (0.22 mL, 2.70 mmol, 2.00 equiv.) and CH₂Cl₂ (6 ml), then cooled in an ice bath. This round bottom flask was equipped with an addition funnel, which was charged with a solution of acetylsalicyloyl chloride (541 mg, 2.73 mmol, 2.00 equiv.) in CH₂Cl₂ (7.5 mL) prepared in another argon-flushed round bottom flask; the solution was added dropwise over 15 minutes. After 16.5 h, the reaction solvent was removed on a rotary evaporator under reduced pressure. The crude residue was purified by two successive flash column chromatography operations (first 90:10 hexanes/EtOAc, then 95:5 hexanes/EtOAc), which provided INT-D060 as a pale yellow oil (557 mg, 76% yield).

R_{f}: 0.60 (SiO₂, 70:30 hexanes:EtOAc);

¹H (300 MHz, CDCl₃): δ 8.02 (dd, J=7.9, 1.4 Hz, 1H), 7.55 (td, J=7.6, 1.3 Hz, 1H), 7.31 (t, J=7.6 Hz, 1H), 7.10 (d, J=7.9 Hz, 1H), 5.54-5.40 (m, 1H), 5.40-5.26 (m, 1H), 4.89 (quint., J=6.2 Hz, 1H), 4.27 (t, J=6.7 Hz, 2H), 2.35 (s, 3H), 2.33-2.21 (m, 4H), 2.09-1.96 (m, 2H), 1.74 (quint., J=7.1 Hz, 2H), 1.62 (quint., J=7.3 Hz, 2H), 1.58-1.48 (m, 2H), 1.48-1.16 (m, 22H), 0.97-0.80 (m, 6H).

### Example 20: Synthesis of INT-D061

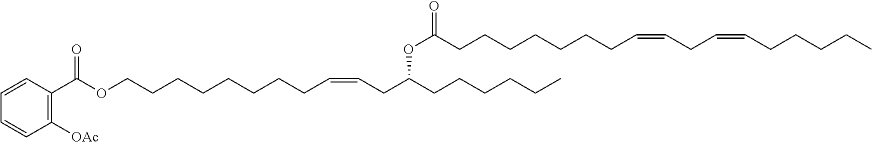 [SMILES: CCCCC/C=C\C/C=C\CCCCCCCC(=O)O[C@@H](C/C=C\CCCCCCCCOC(=O)c1ccccc1OC(C)=O)CCCCCC]

### (R,Z)-12-(Linoleoyloxyoctadec-9-en-1-yl 2-acetoxybenzoate (INT-D061)

HF·pyridine solution (0.39 mL of 70% HF in pyridine, 3.20 mmol, 3.00 equiv.) was added to a stirring, ice-cold THF (5 mL) solution of pyridine (0.26 mL, 3.20 mmol, 3.00 equiv.) and silyl ether 4f (700 mg, 1.06 mmol) in a round bottom flask under argon. When TLC indicated consumption of the starting material (2-8 h), the reaction mixture was quenched with saturated aqueous NaHCO₃. The mixture was extracted with Et₂O (2×10 mL), then the combined organic extracts were washed with H₂O (1×10 ml), brine, dried over Na₂SO₄ and concentrated on a rotary evaporator to afford the crude primary alcohol. The crude was purified by filtration through a plug of silica gel (90:10 hexanes/EtOAc) and concentrated on a rotary evaporator to afford the intermediate primary alcohol (553 mg) as a clear, colourless oil that was used without further purification.

An argon-flushed round bottom flask was charged with the above alcohol (553 mg, 1.01 mmol, 1.00 equiv.), pyridine (0.13 mL, 1.7 mmol, 1.60 equiv.), and CH₂Cl₂ (4 ml), then cooled with an ice bath. A solution of acetylsalicyloyl chloride (327 mg, 1.65 mmol, 1.63 equiv.) in CH₂Cl₂ (6 mL) was prepared in another argon-flushed round bottom flask, and slowly transferred portion-wise into the other round bottom flask via syringe over 15 minutes. After 18 h, the reaction solvent was removed on a rotary evaporator under reduced pressure. The crude residue was purified by two successive flash column chromatography operations (95:5 hexanes/EtOAc), which provided INT-D061 as a pale yellow oil (516 mg, 72% yield).

R_{f}: 0.56 (SiO₂, 70:30 hexanes:EtOAc);

¹H (300 MHz, CDCl₃): δ 8.03 (dd, J=7.9, 1.5 Hz, 1H), 7.57 (td, J=7.6, 1.6 Hz, 1H), 7.32 (td, J=7.6, 1.0 Hz, 1H), 7.11 (d, J=7.9 Hz, 1H), 5.54-5.27 (m, 6H), 4.90 (quint., J=6.2 Hz, 1H), 4.28 (t, J=6.7 Hz, 2H), 2.79 (t, J=5.9 Hz, 2H), 2.36 (s, 3H), 2.34-2.21 (m, 4H), 2.12-1.96 (m, 6H), 1.75 (quint., J=7.1 Hz, 2H), 1.69-1.49 (m, 4H), 1.49-1.18 (m, 32H), 0.98-0.81 (m, 6H).

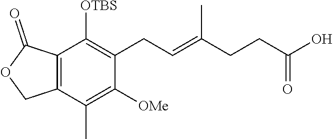 [SMILES: COc1c(C)c2c(c(O[Si](C)(C)C(C)(C)C)c1C/C=C(\C)CCC(=O)O)C(=O)OC2]

### (E)-6-(4-tert-Butyldimethylsilyloxy-6-methoxy-7-methyl-3-oxo-1,3-dihydroisobenzofuran-5-yl)-4-methylhex-4-enolic acid (14)

A flame-dried and argon-flushed round bottom flask was charged with DMF (0.98 mL), imidazole (159 mg, 2.34 mmol, 7.50 equiv.) and mycophenolic acid (100 mg, 0.312 mmol, 1.00 equiv.) and to this mixture was added TBSCl (282 mg, 1.87 mmol, 6.00 equiv.). After 1 h, the reaction mixture was extracted with Et₂O (2×10 mL) from (10 mL). The combined organics were washed with water (340 mL), brine (1×10 mL), dried over Na₂SO₄, an concentrated on a rotary evaporator under reduced pressure. The crude residue was taken up in THF (0.60 mL) and stirred with water (0.60 mL) and acetic acid (0.60 mL) for 1 h. The mixture was then extracted with Et₂O (240 mL) from water (1×10 mL). The combined organics were washed with water (5×10 mL), brine (1×10 ml), dried over Na₂SO₄ and concentrated on a rotary evaporator under reduced pressure. The crude residue was purified by flash column chromatography (80:20→20:80 hexanes/EtOAc) to provide mycophenolic acid silyl ether (14) as a white solid (121 mg, 89% yield).

R_{f}: 0.36 (SiO₂, 50:50 hexanes:EtOAc);

¹H (300 MHz, CDCl₃): δ 5.23 (t, J=6.3 Hz, 1H), 5.09 (s, 2H), 3.76 (s, 3H), 3.41 (d, J=6.3 Hz, 2H), 2.50-2.39 (m, 2H), 2.38-2.27 (m, 2H), 2.17 (s, 3H), 1.78 (s, 3H), 1.05 (s, 9H), 0.26 (s, 6H).

### Example 2P: Synthesis of INT-D062

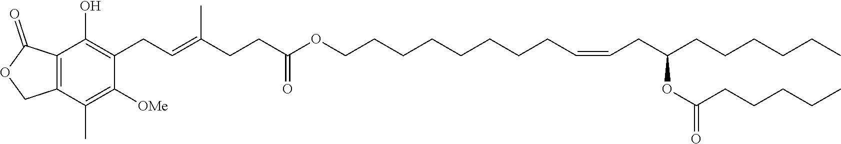 [SMILES: CCCCCC[C@H](C/C=C\CCCCCCCCOC(=O)CC/C(C)=C/Cc1c(O)c2c(c(C)c1OC)COC2=O)OC(=O)CCCCC]

### (12R)-Hexanoyloxyoleyl (E) 6-(4-hydroxy-6-methoxy-7-methyl-3-oxo-1,3-dihydroisobenzofuran-5-yl)-4-methylhex-4-enoate (INT-D062)

HF·pyridine solution (0.11 mL of 70% HF in pyridine, 0.91 mmol, 3.00 equiv.) was added to a stirring, ice-cold THF (1.5 mL) solution of pyridine (0.07 mL, 0.91 mmol, 3.00 equiv.) and silyl ether 4b (150 mg, 0.30 mmol) in a round bottom flask under argon. When TLC indicated consumption of the starting material (2-8 h), the reaction mixture was quenched with saturated aqueous NaHCO₃. The mixture was extracted with Et₂O (2×5 mL), then the combined organic extracts were washed with H₂O (1×5 ml), brine, dried over Na₂SO₄ and concentrated on a rotary evaporator to afford the crude primary alcohol. The crude was purified by filtration through a plug of silica gel (90:10 hexanes/EtOAc) and concentrated on a rotary evaporator to afford the intermediate primary alcohol (102 mg) as a clear, colourless oil that was used without further purification.

An argon-flushed round bottom flask cooled in an ice bath was charged with CH₂Cl₂ (1.2 ml) and mycophenolic acid silyl ether (14) (105 mg, 0.24 mmol, 1.00 equiv.). To this flask was added DCC (50 mg, 0.24 mmol, 1.00 equiv.) and the ice bath was removed. After 15 minutes, the ice bath was replaced under the flask and a solution of the above alcohol (102 mg, 0.267 mmol, 1.10 equiv.) and DMAP (44 mg, 0.36 mmol, 1.50 equiv.) in CH₂Cl₂ (1.2 mL) was added. After 15.5 h, the reaction mixture was concentrated under reduced pressure. The crude residue was diluted with hexanes (4 volumes), filtered through Celite®, then concentrated on a rotary evaporator under reduced pressure. The residue was subjected to flash column chromatography (85:15 hexanes/EtOAc) and the product-containing fractions combined and concentrated.

The residue was transferred into a round bottom flask and flushed with argon. CH₂Cl₂ (1.5 mL) and pyridine (0.06 mL, 0.7 mmol, 2.88 equiv.) were added to this flask and cooled with an ice water bath. Benzoyl chloride (0.05 mL, 0.50 mmol, 2.06 equiv.) was then added to the flask. After 18 h, the reaction mixture was concentrated on a rotary evaporator under reduced pressure. The crude residue was extracted with Et₂O (3×10 mL) and water (1×10 mL). The combined organics were washed with aq. 1 M HCl (1×10 mL), aq. 1 M NaOH (1×10 mL), water (1×10 mL), brine (1×10 mL), dried over Na₂SO₄ and concentrated on a rotary evaporator under reduced pressure.

The crude residue was taken up in THF (1.4 mL) in a round bottom flask that was flushed with argon and cooled with an ice water bath. To this flask was added pyridine (0.07 mL, 0.80 mmol, 3.29 equiv.) and HF·pyridine (0.10 mL of 70% HF, 0.83 mmol, 3.42 equiv.), and the ice bath was removed. After 2 h, saturated aqueous NaHCO₃ was added slowly into the reaction solution until bubbling had stopped. The reaction mixture was extracted with Et₂O (1×10 mL) and the combined organic layers were washed with aq. 1 M HCl (1×10 mL), water (1×10 mL), brine (1×10 mL), dried over Na₂SO₄ and concentrated on a rotary evaporator under reduced pressure. The resulting residue was purified by flash column chromatography (90:10→80:20 hexanes/EtOAc) which provided INT-D062 as a clear, colourless oil (100 mg, 60% yield over 3 steps).

R_{f}: 0.22 (silica, 80:20 hexanes:EtOAc);

¹H (300 MHz, CDCl₃): δ 7.59 (s, 1H), 5.55-5.41 (m, 1H), 5.41-5.17 (m, 4H), 4.90 (quint., J=6.2 Hz, 1H), 4.02 (t, J=6.8 Hz, 2H), 3.78 (s, 3H), 3.40 (d, J=6.9 Hz, 2H), 2.47-2.36 (m, 2H), 2.36-2.23 (m, 6H), 2.17 (s, 3H), 2.10-1.97 (m, 2H), 1.82 (s, 3H), 1.71-1.47 (m, 6H), 1.43-1.18 (m, 22H), 0.97-0.83 (m, 6H).

### Example 2Q: Synthesis of INT-D053

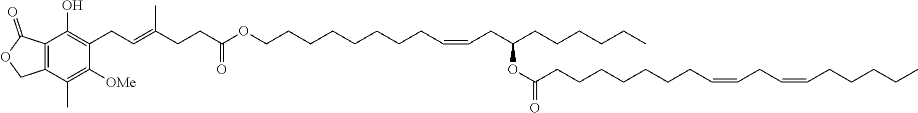 [SMILES: CCCCC/C=C\C/C=C\CCCCCCCC(=O)O[C@@H](C/C=C\CCCCCCCCOC(=O)CC/C(C)=C/Cc1c(O)c2c(c(C)c1OC)COC2=O)CCCCCC]

### (12R)-Linoleoyloxyoleyl (E)-6-(4-hydroxy-6-methoxy-7-methyl-3-oxo-1,3-dihydroisobenzofuran-5-yl)-4-methylhex-4-enoate (INT-D063)

HF-pyridine solution (0.07 mL of 70% HF in pyridine, 0.60 mmol, 3.00 equiv.) was added to a stirring, ice-cold THF (1.5 ml) solution of pyridine (0.05 ml, 0.60 mmol, 3.00 equiv.) and silyl ether 4f (125 mg, 0.19 mmol) in a round bottom flask under argon. When TLC indicated consumption of the starting material (2-8 h), the reaction mixture was quenched with saturated aqueous NaHCO₃. The mixture was extracted with Et₂O (2×10 mL), then the combined organic extracts were washed with H₂O (1×10 mL), brine, dried over Na₂SO₄ and concentrated on a rotary evaporator to afford the crude primary alcohol. The crude was purified by filtration through a plug of silica gel (90:10 hexanes/EtOAc) and concentrated on a rotary evaporator to afford the intermediate primary alcohol (95 mg) as a clear, colourless oil that was used without further purification.

An argon-flushed round bottom flask cooled in an ice bath was charged with CH₂Cl₂ (0.5 mL) and mycophenolic acid silyl ether (14) (68 mg, 0.16 mmol, 1.00 equiv.). To this flask was added DCC (32 mg, 0.16 mmol, 1.00 equiv.) and the ice bath was removed. After 15 minutes, the ice bath was replaced under the flask and a solution of the above alcohol and DMAP (29 mg, 0.24 mmol, 1.50 equiv.) in CH₂Cl₂ (1 mL) was added. After 19 h, the reaction mixture was concentrated under reduced pressure. The crude residue was diluted with hexanes (4 volumes), filtered through Celite®, then concentrated on a rotary evaporator under reduced pressure. The residue was subjected to flash column chromatography (85:15 hexanes/EtOAc) and the product-containing fractions combined and concentrated.

The residue was transferred into a round bottom flask and flushed with argon. CH₂Cl₂ (1 ml) and pyridine (0.03 ml, 0.40 mmol, 2.50 equiv.) were added to this flask and cooled with an ice water bath. Benzoyl chloride (0.03 ml, 0.20 mmol, 1.25 equiv.) was then added to the flask. After 18 h, the reaction mixture was concentrated on a rotary evaporator under reduced pressure. The crude residue was extracted with Et₂O (3×10 mL) and water (1×10 ml). The combined organics were washed with aq. 1 M HCl (1×10 mL), aq. 1 M NaOH (1×10 ml), water (1×10 ml), brine (1×10 mL), dried over Na₂SO₄ and concentrated on a rotary evaporator under reduced pressure.

The crude residue was taken up in THF (1 m) in a round bottom flask that was flushed with argon and cooled with an ice water bath. To this flask was added pyridine (0.04 ml, 0.50 mmol, 3.13 equiv.) and HF·pyridine (0.06 mL of 70% HF, 0.50 mmol, 3.13 equiv.), and the ice bath was removed. After 2 h, saturated aqueous NaHCO₃ was added slowly into the reaction solution until bubbling had stopped. The reaction mixture was extracted with Et₂O (1×10 ml) and the combined organic layers were washed with aq. 1 M HCl (1×10 mL), water (110 ml), brine (1×10 mL), dried over Na₂SO₄ and concentrated on a rotary evaporator under reduced pressure. The resulting residue was purified by flash column chromatography (90:10 hexanes/EtOAc) which provided INT-0063 as a clear, colourless oil (66 mg, 50% yield over 3 steps).

R_{f}: 0.17 (SiO₂, 85:15 hexanes:EtOAc);

¹H (300 MHz, CDCl₃): δ 7.69 (s, 1H), 5.55-5.17 (m, 9H), 4.90 (quint., J=6.3 Hz, 1H), 4.02 (t, J=6.7 Hz, 2H), 3.78 (s, 3H), 3.40 (d, J=6.7 Hz, 2H), 2.79 (t, J=6.0 Hz, 2H), 2.46-2.36 (m, 2H), 2.36-2.23 (m, 6H), 2.17 (s, 3H), 2.13-1.96 (m, 6H), 1.82 (s, 3H), 1.70-1.47 (m, 6H), 1.45-1.19 (m, 32H), 0.96-0.81 (m, 6H).

### Example 2R: Synthesis f INT-D065

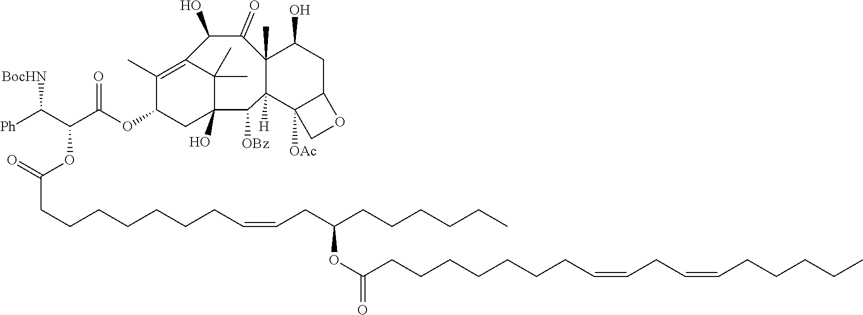

### (4S,4aS,6R,9S,11S,12S,12aR,12bS)-12b-acetoxy-9-(((2R,3S)-3-((tert-butoxycarbonyl)amino)-2-(((R,Z)-12-(((9Z,12Z)-octadeca-9,12-dienoyl)oxy)octadec-9-enoyl)oxy)-3-phenylpropanoyl)oxy)-4,6,11-trihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-12-yl benzoate (INT-D065)

Et₃N (0.10 ml, 0.75 mmol, 2.50 equiv.), followed by the Mukaiyama reagent (100 mg, 0.39 mmol, 1.30 equiv.), were added to a room temperature CH₂Cl₂ (3 mL) solution of docetaxel (242 mg, 0.30 mmol) and 12R-linoleoyloxyoleic acid 7b (202 mg, 0.36 mmol, 1.20 equiv.) in a round bottom flask under argon. After stirring for 14 h, the reaction mixture was diluted with EtOAc, filtered through Celite® and concentrated on a rotary evaporator under reduced pressure. The crude residue was purified by flash column chromatography (SiO₂, 80:20→50:50 hexanes/EtOAc) to afford INT-D065 as a clear, colourless oil (243 mg, 60% yield).

R_{f}=0.45 (SiO₂, 50:50 hexanes/EtOAc);

¹H NMR (300 MHz, CDCl₃): δ 8.12 (d, J=7.3 Hz, 2H), 7.62 (t, J=7.4 Hz, 1N), 7.56-7.46 (m, 2H), 7.44-7.35 (m, 2H) 7.35-7.26 (m, 3H), 6.27 (br t, J=8.0 Hz, 1H), 5.70 (d, J=7.1 Hz, 1H), 5.55-5.27 (m, 9H), 5.23 (s, 1H), 4.98 (m, 1H), 4.90 (quint., J=6.2 Hz, 1H), 4.39-4.16 (m, 4H), 3.95 (d, J=6.9 Hz, 1H), 2.79 (t, J=5.8 Hz, 2H), 2.67-2.52 (m, 1H), 2.46 (s, 3H), 2.44-2.23 (m, 8H), 2.22-1.71 (m, 18H), 1.70-1.45 (m, 7H), 1.44-1.12 (m, 45H), 1.13 (s, 3H), 0.97-0.82 (m, 6H).

### Example 2S: Synthesis of INT-D053

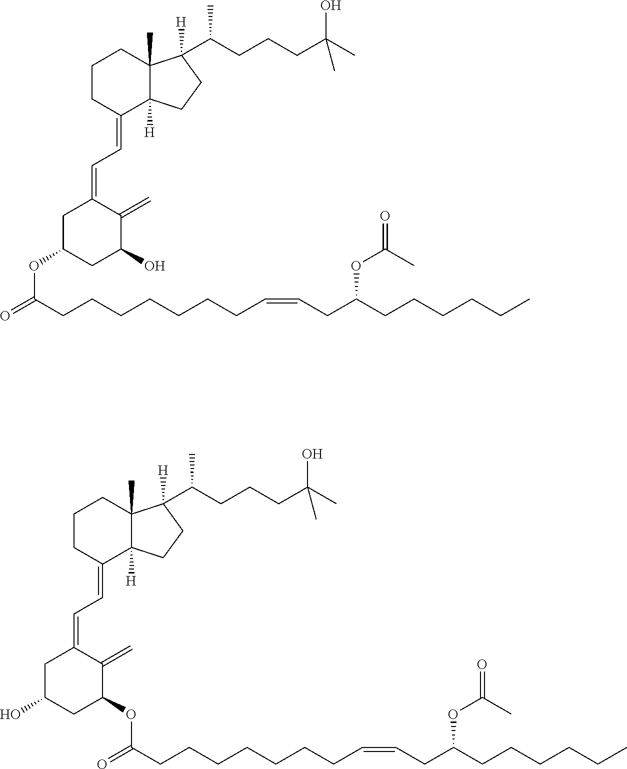 [SMILES: C=C1/C(=C\C=C2/CCC[C@]3(C)[C@@H]([C@H](C)CCCC(C)(C)O)CC[C@@H]23)C[C@@H](OC(=O)CCCCCCC/C=C\C[C@@H](CCCCCC)OC(C)=O)C[C@@H]1O ; C=C1/C(=C\C=C2/CCC[C@]3(C)[C@@H]([C@H](C)CCCC(C)(C)O)CC[C@@H]23)C[C@@H](O)C[C@@H]1OC(=O)CCCCCCC/C=C\C[C@@H](CCCCCC)OC(C)=O]

### (1R,3S,2)-3-hydroxy-5-(2-((1R,3aS,7aR,E)-1-((R)-6-hydroxy-6-methylheptan-2-yl)-7a-methyloctahydro-4H-inden-4-ylidene)ethylidene)-4-methylenecyclohexyl (R,Z)-12-acetoxyoctadec-9-enoate and (1S,5R,Z)-5-hydroxy-3-(2-((1R,3aS,7aR,E)-1-((R)-6-hydroxy-6-methylheptan-2-yl)-7a-methyloctahydro-4H-inden-4-ylidene)ethylidene)-2-methylenecyclohexyl(RA-12-acetoxyoctadec-9-enoate (INT-D053): JZ-25-057, 029

DCC (50 mg, 0.24 mmol, 1.20 equiv.) was added to a stirring, ice-cold 1:1 CH₂Cl₂/THF (4 mL) solution of (12R)-acetoxyoleic acid (82 mg, 0.24 mmol, 1.20 equiv.) in a round bottom flask under argon, then the ice bath was removed and the resultant stirred for 15 min. The reaction mixture was cooled again in an ice bath and solid calcitriol (83 mg, 0.20 mmol) and DMAP (29 mg, 0.24 mmol, 1.20 equiv.) were added. The reaction mixture was allowed to warm up over 14 h, diluted with EtOAc, stirred for 10 min, then filtered through Celite®. The filtrate was concentrated to afford the crude as a pale yellow oil and subsequently purified by flash column chromatography (SiO₂, 80:20→65:35 hexanes/EtOAc) to afford an ˜1.1 mixture of the 1- and 3-acylated conjugates (61 mg, 41% yield) as a clear, colourless oil.

R_{f}=0.33 (SiO₂, 60:40 hexanes/EtOAc);

¹H NMR (300 MHz, CDCl₃): δ 6.44-6.25 (m, 2H), 6.02 (d, J=11.2 Hz, 1H), 5.92 (d, J=11.2 Hz, 1H), 5.56-5.40 (m, 3H), 5.40-5.27 (m, 4H), 5.26-5.16 (m, 1H), 5.07-4.97 (m, 2H), 4.87 (quint., J=6.2 Hz, 2H), 4.45-4.34 (m, 1H), 4.23-4.10 (m, 1H), 2.89-2.74 (m, 2H), 2.68-2.51 (m, 21H), 2.48-2.18 (m, 11H), 2.17-1.77 (m, 25H), 1.76-1.13 (m, 90H), 1.12-0.99 (m, 2H), 0.99-0.80 (m, 13H), 0.55 (s, 3H), 0.52 (s, 3H).

### Example 2T: Synthesis of INT-D068

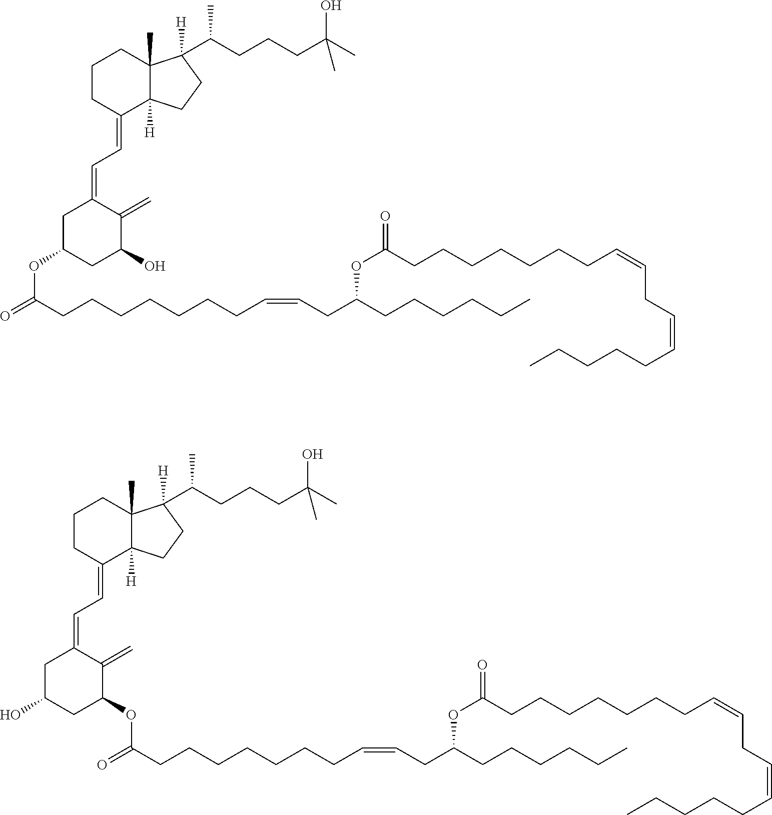 [SMILES: C=C1/C(=C\C=C2/CCC[C@]3(C)[C@@H]([C@H](C)CCCC(C)(C)O)CC[C@@H]23)C[C@@H](OC(=O)CCCCCCC/C=C\C[C@@H](CCCCCC)OC(=O)CCCCCCC/C=C\C/C=C\CCCCC)C[C@@H]1O ; C=C1/C(=C\C=C2/CCC[C@]3(C)[C@@H]([C@H](C)CCCC(C)(C)O)CC[C@@H]23)C[C@@H](O)C[C@@H]1OC(=O)CCCCCCC/C=C\C[C@@H](CCCCCC)OC(=O)CCCCCCC/C=C\C/C=C\CCCCC]

### (R,Z)-18-(((1R,3S,Z)-3-Hydroxy-S-(2-((1R,3aS,7aR,E)-1-((R)-6-hydroxy-6-methylheptan-2-yl)-7a-methyloctahydro-4H-inden-4-ylidene)ethylidene)-4-methylenecyclohexyl)oxy)-18-oxooctadec-9-en-7-yl linoleate and (R,Z)-18-(((1S,5R,Z)-5-hydroxy-3-(2-((1R,3aS,7aR,E)-1-((R)-6-hydroxy-6-methylheptan-2-yl)-7a-methyloctahydro-4H-inden-4-ylidene)ethylidene)-2-methylenecyclohexyl)oxy)-18-oxooctadec-9-en-7-yl linoleate (INT-D068)

DCC (50 mg, 0.24 mmol, 1.20 equiv.) was added to a stirring, ice-cold 1:1 CH₂Cl₂/THF (4 ml) solution of (12R)-linoleoyloxyoleic acid (135 mg, 0.24 mmol, 1.20 equiv.) in a round bottom flask under argon, then the ice bath was removed and the resultant stirred for 15 min. The reaction mixture was cooled again in an ice bath and solid calcitriol (83 mg, 0.20 mmol) and DMAP (29 mg, 0.24 mmol, 1.20 equiv.) were added. The reaction mixture was allowed to warm up over 14 h, diluted with EtOAc, stirred for 10 min, then filtered through Celite®. The filtrate was concentrated to afford the crude as a pale yellow oil and subsequently purified by flash column chromatography (SiO₂, 95:5+90:10→70:30 hexanes/EtOAc) to afford an ˜1:1 mixture of the 1- and 3-acylated conjugates (75 mg, 39% yield) as a clear, colourless oil.

R_{f}=0.26 (SiO₂, 70:30 hexanes/EtOAc);

¹H NMR (300 MHz, CDCl₃): δ 6.43-5.26 (m, 2H), 6.02 (d, J=11.2 Hz, 1H), 5.92 (d, J=11.2 Hz, 1H), 5.57-5.26 (m, 15H), 5.26-5.16 (m, 1H), 5.07-4.97 (m, 2H), 4.88 (quint., J=6.2 Hz, 2H), 4.47-4.34 (m, 1H), 4.23-4.10 (m, 1H), 2.89-2.70 (m, 6H), 2.68-2.52 (m, 2H), 2.47-2.20 (m, 15H), 2.16-1.77 (m, 25H), 1.77-1.12 (m, 118H), 1.12-1.01 (m, 2H), 1.00-0.79 (m, 19H), 0.55 (s, 3H), 0.52 (s, 3H).

### Example 2U: Synthesis of INT-D070

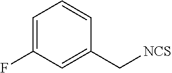

### 3-Fluorobenzylisothiocyanate (15)

Et₃N (2.75 mL, 3.30 mmol, 3.30 equiv.) was added to an ice-cold THF (10 mL) solution of 3-fluorobenzylamine (750 mg, 6.00 mmol) in a round bottom flask under argon. A THF (10 mL) solution of carbon disulfide (0.45 mL, 7.20 mmol, 1.20 equiv.) was then added via syringe pump over 30 minutes. The reaction mixture was allowed to warm to room temperature and after 3 h, the mixture was again cooled in an ice bath and tosyl chloride (1.26 g, 7.20 mmol, 1.20 equiv.) was added. After an additional 3 h, aqueous 1 M HCl (10 mL) was added and the reaction mixture was extracted with ethyl acetate (3×10 ml). The combined organics were washed with brine, dried over Na₂SO₄ and concentrated on a rotary evaporator under reduced pressure. The crude product was purified by flash column chromatography (98:2→96:4 hexanes/EtOAc) to afford isothiocyanate 15 (846 mg, 84% yield) as a clear, colourless oil.

R_{f}=0.45 (SiO₂, 80:20 hexanes/EtOAc);

¹H NMR (300 MHz, CDCl₃): δ 7.42-7.35 (m, 1H), 7.13-7.04 (m, 3H), 7.74 (s, 2H)

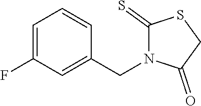 [SMILES: O=C1CSC(=S)N1Cc1cccc(F)c1]

### 3-(3-Fluorobenzyl)-2-thioxothiazolidin-4-one (16)

Thioglycolic acid (0.17 mL, 2.40 mmol, 0.75 equiv.) was added to an ice-cold mixture of Et₃N (0.90 mmol, 6.40 mmol, 2.00 equiv.) and water (10 mL) in a round bottom flask under argon and a THF (5 ml) solution of isothiocyanate 15 (539 mg, 3.20 mmol) was added over 5 minutes. The reaction mixture was allowed to warm to room temperature until had turned light orange in colour. The mixture was adjusted to pH ≤2 by addition of aqueous 6 M HCl. The reaction mixture was heated at reflux for 14 h, then cooled to room temperature and extracted with EtOAc (3×10 mL). The combined organics were washed with brine, dried over anhydrous sodium sulfate and concentrated on a rotary evaporator under reduced pressure. The crude product purified by filtration through silica gel (50:50 hexanes/EtOAc) to afford rhodanine 16 (466 mg, 80% yield) as a yellow solid.

R_{f}=0.52 (SiO₂, 70:30 hexanes/EtOAc);

¹H NMR (300 MHz, CDCl₃): δ 7.72 (s, 1H), 7.65 (d, J=7.7 Hz, 1H), (d, J=7.7 Hz, 1H), 7.46 (t, J=7.8 Hz, 2H), 5.25 (s, 2H), 4.04 (s, 2H).

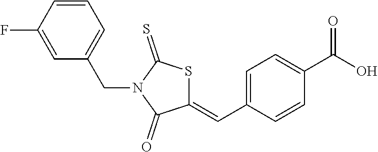 [SMILES: O=C(O)c1ccc(/C=C2\SC(=S)N(Cc3cccc(F)c3)C2=O)cc1]

### (Z)-4-((3-(3-Fluorobenzyl)-4-oxo-2-thioxothiazolidin-5-ylidene)methyl)benzoic acid (INT-MA014)

4-Carboxybenzaldehyde (151 mg, 1.01 mmol, 1.10 equiv.) was added to an EtOH (4 mL) solution of rhodanine 16 (221 mg, 0.92 mmol) and piperidine (0.01 mL, 0.14 mmol, 0.15 equiv.) in a round bottom flask, then heated at reflux. After 1.5 h, the reaction mixture was concentrated on a rotary evaporator under reduced pressure. The crude was then filtered through silica gel (90:8:2 CH₂Cl₂/MeOH/HOAc) and concentrated under reduced pressure. Precipitation from hot EtOH afforded rhodanine carboxylic acid INT-MA014 (179 mg, 52% yield) as a yellow solid.

R_{f}=0.26 (SiO₂, 50:40:10 hexanes/EtOAc/MeOH);

¹H NMR (300 MHz, CDCl₃): δ 8.08 (d, J=8.2 Hz, 2H), 7.91 (s, 1H), 7.77 (d, J=8.3 Hz, 2H), 7.43-7.36 (m, 1H), 7.20-7.11 (m, 3H), 5.27 (s, 2H).

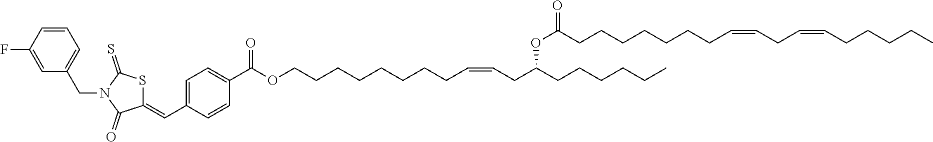 [SMILES: CCCCC/C=C\C/C=C\CCCCCCCC(=O)O[C@@H](C/C=C\CCCCCCCCOC(=O)c1ccc(/C=C2\SC(=S)N(Cc3cccc(F)c3)C2=O)cc1)CCCCCC]

### 12R-linoleoyloxyoleyl 4-((Z)-(3-(3-fluorobenzyl)-4-oxo-2-thioxothiazolidin-5-ylidene)methyl)benzoate (INT-D070)—JZ-25-169

i-Pr₂NEt (0.37 mL, 2.10 mmol, 1.50 equiv.), then BOP reagent (682 mg, 1.54 mmol, 1.10 equiv.), was added to a room temperature DMF (3.5 mL) solution of carboxylic acid INT-MA014 (523 mg, 1.40 mmol) and 12R-linoleoyloxyoleyl alcohol (843 mg, 1.54 mmol, 1.10 equiv.) in a round bottom flask under argon. After stirring for 14 h, the reaction mixture was diluted with water and extracted with t-BuOMe (3×15 mL). The combined organic layers were washed with water (4×10 mL), brine (3×10 ml), dried over Na₂SO₄ and concentrated on a rotary evaporator under reduced pressure. The crude residue was purified by flash column chromatography (SiO₂, 99:1→95:5 hexanes/EtOAc) to afford INT-D070 as a yellow oil (1.18 g, 93% yield).

R_{f}=0.47 (SiO₂, 90:10 hexanes/EtOAc);

¹H NMR (300 MHz, CDCl₃): δ 8.15 (d, J=8.3 Hz, 2H), 7.78 (s, 1H), 7.58 (d, J=8.2 Hz, 2H), 7.37-7.26 (m, 2H), 7.23-7.15 (m, 1H), 7.06-6.96 (m, 1H), 5.55-5.23 (m, 8H), 4.90 (quint., J=6.2 Hz, 1H), 4.36 (t, J=6.7 Hz, 2H), 2.79 (t, J=5.9 Hz, 2H), 2.36-2.22 (m, 4H), 2.15-1.96 (m, 6H), 1.79 (m, 2H), 1.70-1.14 (m, 36H), 0.98-0.80 (m, 6H).

### Example 2V: Synthesis of INT-H001

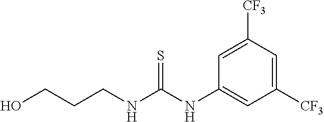 [SMILES: OCCCNC(=S)Nc1cc(C(F)(F)F)cc(C(F)(F)F)c1]

### 1-(3,5-Bis(trifluoromethyl)phenyl)-3-(3-hydroxypropyl)thiourea (15): JZ-25-145

3-Aminopropanol (0.33 mL, 4.40 mmol, 1.10 equiv.) was added to a room temperature MeCN (8 mL) solution of 3,5-bis(trifluoromethyl)phenyl isothiocyanate (1.08 g, 4.00 mmol) and Et₃N (0.61 ml, 4.40 equiv., 1.10 equiv.) in a round bottom flask under argon. After 14 h, the reaction mixture was diluted with H₂O and extracted with EtOAc (3×15 mL). The combined organic layers were washed with H₂O (1×15 ml), brine, dried over Na₂SO₄ and concentrated on a rotary evaporator under reduced pressure. The crude semi-solid was filtered through a plug of silica gel (75:25 EtOAc/hexanes), the filtrate concentrated under reduced pressure, then recrystallized from t-BuOMe/hexanes to afford thiourea 15 (1.18 g, 85% yield) as a white solid.

¹H NMR (300 MHz, DMSO-de): δ 10.1 (br s, 1H), 8.26 (br s, 3H), 7.72 (br s, 1H), 4.59 (br s, 1H), 3.66-3.39 (m, 4H), 1.72 (quint., J=6.4 Hz, 2H);

¹³C NMR (75.5 MHz, DMSO-de): δ 180.4, 142.0, 130.1 (q, J=34 Hz), 123.3 (q, J=273 Hz), 121.7 (br), 115.9 (br), 58.7, 41.6, 31.3.

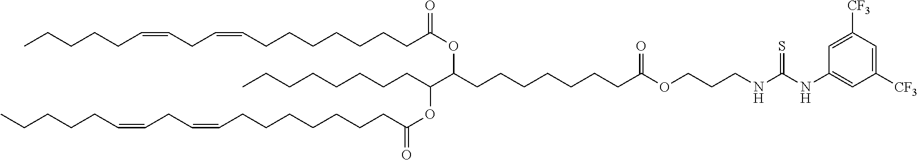 [SMILES: CCCCC/C=C\C/C=C\CCCCCCCC(=O)OC(CCCCCCCC)C(CCCCCCCC(=O)OCCCNC(=S)Nc1cc(C(F)(F)F)cc(C(F)(F)F)c1)OC(=O)CCCCCCC/C=C\C/C=C\CCCCC]

### 1-(3,5-Bis(trifluoromethyl)phenyl)-3-(3-hydroxypropyl)thiourea (INT-H001)

DCC (68 mg, 0.33 mmol, 1.10 equiv.) was added to a stirring, ice-cold CH₂Cl₂ (3 mL) solution of carboxylic acid 12b (252 mg, 0.30 mmol, 1.10 equiv.) in a round bottom flask under argon, then the ice bath was removed and the resultant stirred for 15 min. The reaction mixture was cooled again in an ice bath and thiourea 15 (114 mg, 0.33 mmol) and DMAP (44 mg, 0.36 mmol, 1.20 equiv.) were added. The reaction mixture was allowed to warm up over 14 h, diluted with t-BuOMe, stirred for 10 min, then filtered through Celite®. The filtrate was concentrated to afford the crude as a pale yellow oil and subsequently purified by flash column chromatography (SiO₂, 80:18:2 hexanes/EtOAc/MeOH) to afford INT-H001 (222 mg, 63% yield) as a clear, colourless oil.

R_{f}=0.35 (SiO₂, 80:18:2 hexanes/EtOAc/MeOH);

¹H NMR (300 MHz, CDCl₃): δ 8.06 (br s, 1H), 7.88 (s, 2H), 7.72 (s, 1H), 6.90 (br t, 1H), 5.49-5.24 (m, 8H), 5.10-4.90 (m, 2H), 4.20 (t, J=5.6 Hz, 2H), 3.79-3.64 (m, 2H), 2.79 (t, J=5.9 Hz, 4H), 2.29 (m, 6H), 2.14-1.93 (m, 10H), 1.70-1.45 (m, 10H), 1.45-1.16 (m, 48H), 0.96-0.83 (m, 9H).

### Example 2W: Synthesis of Disubstituted Calcitriol, INT-D087

An example of a synthesis scheme for preparing a calcitriol lipid conjugate disubstituted with two lipid moieties is provided below:

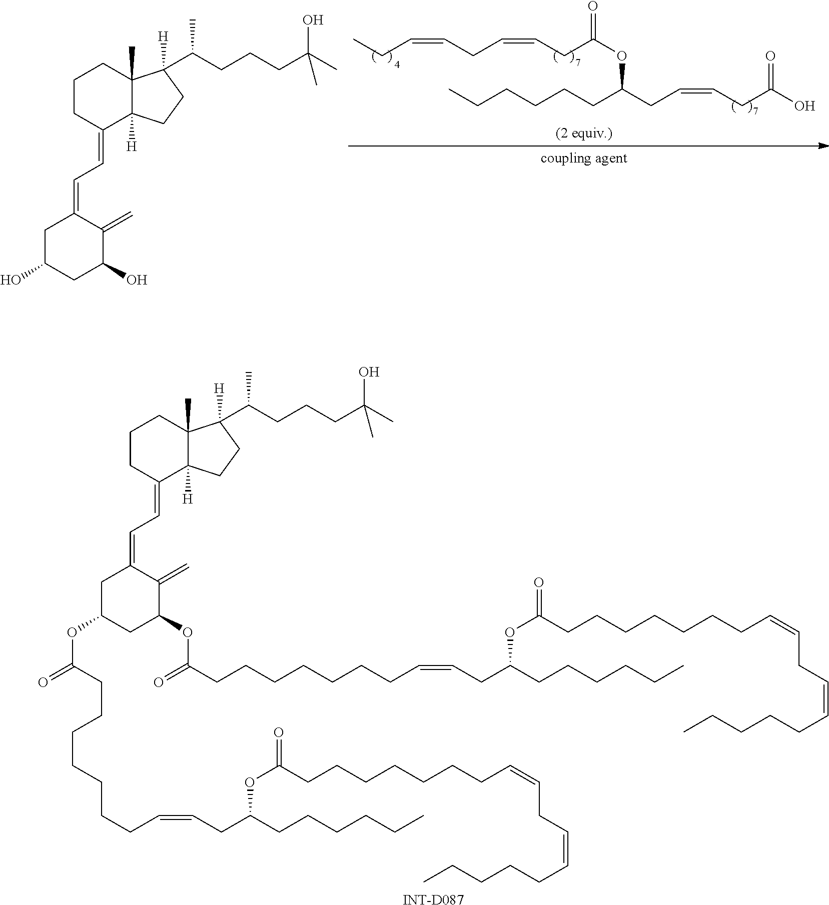

### Example 3: Formulation of Pro-Drug in a Lipid Nanoparticle (LNP)

The lipid-like properties of the pro-drugs allow them to be easily loaded in LNP systems by simply mixing them with the lipid formulation components. That is, loading can be achieved in some embodiments without any further modification of known formulation processes. As a result, an LNP incorporating these drug-lipid conjugates can be made using a wide variety of well described formulation methodologies including, but not limited to, extrusion, ethanol injection and in-line mixing.

LNPs were prepared by dissolving 1,2-Distearoyl-sn-glycero-3-phosphocholine (DSPC) or 1,2-dimyristoyl-sn-glycero-3-phosphocholine (DMPC), cholesterol and 1,2-Distearoyl-sn-glycero-3-phosphoethanolamine-Poly(ethylene glycol) (PEG-DSPE) in ethanol. DSPC, DMPC and PEG-DSPE were purchased from Avanti Polar Lipids (Alabaster, Ala.), and cholesterol was obtained from Sigma (St Louis, Mo.).

Drug-lipid-conjugates INT-D034, INT-D035, INT-D045, INT-D046, INT-D047, INT-D048, INT-D049, INT-D050, INT-D051, INT-D053, INT-D060, INT-D061, INT-D062, INT-D063, INT-D083, INT-D085, INT-D086, INT-D088 and INT-D089 (see FIG. 3 and Example 7 for structures) were dissolved in isopropanol or THF. LNP were prepared by rapidly mixing DSPC or DMPC, cholesterol, drug-lipid conjugate, and PEG-DSPE (in molar ratio of 49/40/10/1) with phosphate-buffered saline (PBS) using a cross-junction mixer. Formulations were dialyzed against PBS to remove residual ethanol. In formulation$ with more than 10 mol % drug-lipid conjugates, the amount of phospholipid or cholesterol was reduced accordingly.

The physiochemical properties of the LNPs prepared as described above were subsequently characterized. Particle size was determined by dynamic light scattering using a Malvern Zetasizer Nano ZS (Malvern, UK) following buffer exchange into phosphate-buffered saline. Number-weighted size and distribution date was used. Lipid concentrations were determined by measuring total cholesterol using the Cholesterol E enzymatic assay kit from Wako Chemicals USA (Richmond, Va.). The morphology of LNP formulations containing LD-DEX was analyzed by cryogenic-transmission electron microscopy (cryoTEM).

Table 4 below shows that the pro-drugs described herein can be formulated in LNPs at high encapsulation efficiency and low polydispersity, both of which are desirable physiochemical properties for drug delivery vehicles.

TABLE 4
Physiochemical parameters of LNP containing
10 mol % ricinoleyl-dexamethasone conjugates

| | | Particle | Polydispersity | Encapsulation |
|---|---|---|---|---|
| Compound | Primary | Diameter | index | Efficiency |
| ID | PC-lipid | (nm) | (PDI) | (%) |
| INT-D034 | DSPC | 50 | 0.047 | 92 |
| INT-D035 | DSPC | 45 | 0.066 | 94 |
| INT-D045 | DSPC | 45 | 0.077 | 90 |
| INT-D046 | DSPC | 46 | 0.055 | 93 |
| INT-D047 | DSPC | 58 | 0.054 | 93 |
| INT-D048 | DSPC | 50 | 0.056 | 88 |
| INT-D049 | DSPC | 53 | 0.036 | 100 |
| INT-D050 | DSPC | 53 | 0.033 | 100 |
| INT-D051 | DSPC | 54 | 0.052 | 100 |
| INT-D034 | DSPC | 70 | 0.08 | 99 |
| INT-D035 | DSPC | 64 | 0.11 | 99 |
| INT-D045 | DSPC | 64 | 0.03 | 100 |
| INT-D046 | DSPC | 64 | 0.08 | 100 |
| INT-D047 | DSPC | 70 | 0.06 | 100 |
| INT-D048 | DSPC | 62 | 0.05 | 98 |
| INT-D049 | DSPC | 65 | 0.11 | 100 |
| INT-D050 | DSPC | 60 | 0.05 | 90 |
| INT-D051 | DSPC | 61 | 0.04 | 100 |
| INT-D034 | DMPC | 72 | 0.02 | 100 |
| INT-D035 | DMPC | 67 | 0.03 | 100 |
| INT-D045 | DMPC | 68 | 0.03 | 99 |
| INT-D046 | DMPC | 69 | 0.04 | 93 |
| INT-D047 | DMPC | 68 | 0.02 | 100 |
| INT-D048 | DMPC | 64 | 0.03 | 100 |
| INT-D049 | DMPC | 68 | 0.05 | 94 |
| INT-D050 | DMPC | 61 | 0.04 | 97 |
| INT-D051 | DMPC | 65 | 0.03 | 84 |

### Example 4: Pro-Drugs Form Monodispersed LNP with Novel Macromolecular Structure

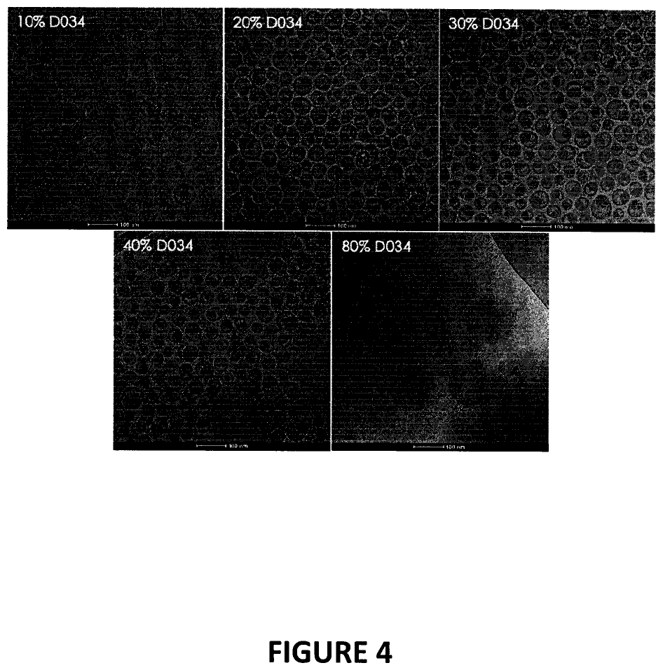

The pro-drug, INT-D034, having a hexanoyl group, (FIG. 3A) was mixed with neutral phospholipid and cholesterol at 0 to 99 mol % pro-drug concentration using a rapid mixing technique set out in Example 3 to produce monodispersed LNP formulations (FIG. 4). All INT-D034 formulations showed high encapsulation efficiencies, with particle diameter at ˜29-87 nm and polydispersity index (PDI) at or less than 0.1 (Table 5 below). Electron micrographs of LNP formulations show enlargement of a globular electron-dense area immediately at the membrane as the amount of INT-D034 Increased, suggesting that the pro-drug INT-0034 exists as a hydrophobic oil phase in the LNP lipid bilayer (FIG. 4).

TABLE 5
Particle size and polydispersity index of LNP containing
various amounts of pro-drugs

| Pro-drug | | | |
|---|---|---|---|
| concentration (%) | Pro-drug | Size (nm) | PdI |
| 0 | | 42 | 0.061 |
| 10 | D034 | 48 | 0.064 |
| 20 | | 45 | 0.068 |
| 30 | | 43 | 0.082 |
| 40 | | 41 | 0.100 |
| 50 | | 32 | 0.085 |
| 60 | | 48 | 0.022 |
| 80 | | 87 | 0.025 |
| 95 | | 29 | 0.082 |
| 98 | | 52 | 0.014 |
| 99 | | 86 | 0.011 |
| 10 | D045 | 46 | 0.076 |
| 20 | | 48 | 0.073 |
| 80 | | 69 | 0.033 |
| 90 | | 74 | 0.054 |
| 99 | | 75 | 0.045 |
| 50 | D049 | 44 | 0.05 |
| 60 | | 61 | 0.02 |
| 80 | | 92 | 0.03 |
| 98 | | 69 | 0.05 |
| 99 | | 103 | 0.03 |
| 80 | D050 | 101 | 0.01 |
| 98 | | 61 | 0.02 |
| 99 | | 105 | 0.01 |
| 80 | D051 | 87 | 0.02 |
| 98 | | 56 | 0.04 |
| 99 | | 93 | 0.01 |
| 10 | D053 | 54 | 0.081 |
| 20 | | 56 | 0.052 |
| 80 | | 106 | 0.029 |
| 99 | | 98 | 0.018 |
| 10 | D060 | 46 | 0.06 |
| 20 | | 42 | 0.116 |
| 60 | | 71 | 0.216 |
| 80 | | 67 | 0.042 |
| 99 | | 98 | 0.034 |
| 10 | D061 | 44 | 0.061 |
| 20 | | 39 | 0.065 |
| 60 | | 72 | 0.186 |
| 80 | | 82 | 0.089 |
| 99 | | 123 | 0.045 |
| 10 | D062 | 42 | 0.085 |
| 20 | | 39 | 0.072 |
| 60 | | 70 | 0.212 |
| 80 | | 68 | 0.021 |
| 99 | | 93 | 0.019 |
| 10 | D063 | 41 | 0.069 |
| 20 | | 41 | 0.056 |
| 60 | | 57 | 0.149 |
| 80 | | 91 | 0.031 |
| 99 | | 119 | 0.028 |
| 10 | D083 | 56 | 0.06 |
| 20 | | 51 | 0.067 |
| 99 | | 133 | 0.026 |
| 10 | D085 | 42 | 0.064 |
| 20 | | 39 | 0.075 |
| 60 | | 56 | 0.059 |
| 80 | | 124 | 0.034 |
| 99 | | 135 | 0.006 |
| 10 | D086 | 38 | 0.082 |
| 20 | | 35 | 0.117 |
| 60 | | 82 | 0.036 |
| 80 | | 165 | 0.028 |
| 99 | | 160 | 0.026 |

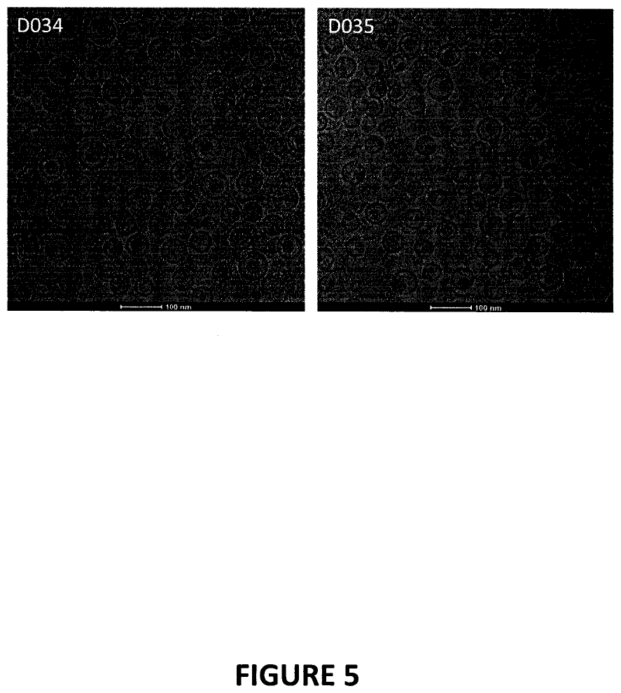

In order to determine if this new ultrastructure is consistent with other ricinoleyl-based conjugates, INT-D035 (having an R hydrocarbon derived from an oleoyl group instead of hexanoyl group in INT D034 as per FIG. 3B) was incorporated in an LNP as described in Example 3 at equivalent amounts of pro-drug (10 mol %). Similar to the INT-D034 formulation, it was observed that the INT-D035 formulation also exhibits a globular electron-dense area immediately at the membrane (FIG. 5). These results indicate that ricinoleyl-based conjugates have the appropriate properties to reside as a hydrophobic oil phase in the LNP lipid bilayer.

Other pro-drugs, including INT-D045, INT-0049, INT-D050, INT-D051, INT-D053, INT-D060, INT-D061, INT-D062, INT-D063, INT-D083, INT-D085 and INT-D086, that contains various R groups can be efficiently incorporated up to 99 mol % in LNP (Table 5).

### Example 5: Dissociation of Pro-Drugs from LNPs as a Function of S Group Hydrophobicity (Log P)

The release of ricinoleyl-dexamethasone conjugates from LNP was examined using an assay involving human plasma, which contains lipoproteins as a lipid sink for lipid exchanges to occur. The plasma lacked active esterases that may digest the ricinoleyl-dexamethasone conjugates, which would in turn prevent the detection and monitoring of intact conjugates.

LNP formulations containing 10-99 mol % INT-D034, INT-D035, INT-D045, INT-D046, INT-0047, INT-D048 or INT-D049, INT-D050, INT-D051, INT-D053, INT-D083, INT-D085, INT-D086 or INT-D089 (see FIG. 3 and Example 7 for structures) were subjected to incubation in human plasma for 0, 2 or 24 hours at 37° C. at 1.2 mM total lipid. Size exclusion chromatography was performed to separate LNP from lipoproteins (1.5×27 cm Sepharose CL-48 size exclusion column). Thirty fractions of 2 mL were collected and three volumes of isopropanol/methanol (1:1 v/v) were added to each fraction.

Drug-lipid conjugates were quantified by ultra high pressure liquid chromatography (UPLC) using a Water® Acquity™ UPLC system equipped with a photodiode array detector (PDA); Empower™ data acquisition software version 2.0 was used (Waters, USA). Separations were performed using a Waters® Acquity™ BEH C18 column (1.7 μm, 2.1×100 mm) at a flow rate of 0.5 ml/min, with a linear gradient from 80/20 (% A/B) to 0/100 (% A/B). Mobile phase A consisted of water and mobile phase B consisted methanol/acetonitrile (1:1, v/v). The method was run over 6 minutes with a column temperature of 55° C. and the analyte was measured by monitoring the PDA detector at a wavelength of 239 nm.

The amount of each intact ricinoleyl-dexamethasone conjugate that remained associated with LNP in each fraction as quantified by UPLC is shown in FIG. 6. Ricinoleyl-dexamethasone conjugates with various Log P values exhibited differential levels of dissociation (FIG. 6). Conjugates with higher predicted Log P values (i.e., more hydrophobic) dissociated from LNP less than those with lower predicted Log P values. For example, over 90% of INT-D086 (Log P of 21.2) remained in LNP, in comparison to ˜40% of INT-D047 (Log P of 8.33) (FIG. 6A and Table 6 below). These results indicate that designing pro-drugs based on the scaffold described herein provides a reliable method to control drug release from an LNP. In situations where the LNP is required to circulate for extended periods in the body system to reach disease sites (such as distal tumours), it is desirable that the drug remains associated with the LNP and does not exhibit premature drug leakage since this may directly correlate with low therapeutic activity.

TABLE 6
Biophysical parameters of LNP formulations containing pro-drugs

| Pro-drug ID | Size of LNP | PdI | % Entrapment | LogP (predicted) |
|---|---|---|---|---|
| INT-D034 | 70 | 0.08 | 99 | 10.37 |
| INT-D035 | 64 | 0.11 | 99 | 15.34 |
| INT-D045 | 64 | 0.03 | 100 | 14.98 |
| INT-D046 | 64 | 0.08 | 100 | 13.04 |
| INT-D047 | 70 | 0.06 | 100 | 8.33 |
| INT-D048 | 62 | 0.05 | 98 | 10.13 |
| INT-D049 | 65 | 0.11 | 100 | 14.62 |
| INT-D050 | 60 | 0.05 | 90 | 15.7 |
| INT-D051 | 61 | 0.04 | 100 | 15.14 |
| INT-D085 | 42 | 0.06 | 100 | 11.98 |
| INT-D086 | 38 | 0.08 | 92 | 21.2 |
| INT-D089 | 41 | 0.07 | 100 | 14.83 |

In order to provide therapeutic activity, the active drug ultimately has to be released from the conjugate. The exemplified ricinoleyl-based conjugates contain a biodegradable, esterase sensitive linker between the active drug and the ricinoleyl scaffold. Mouse plasma was used to examine the biodegradability of ricinoleyl-based conjugates as it contains active esterases that can cleave the linker. LNP formulations containing INT-0034, INT-D035, INT-D045, INT-D046, INT-D047, INT-D048, INT-D049, INT-D050, INT-D051, INT-0053, INT-D083, INT-D085, INT-D086 or INT-D089 were incubated with mouse plasma for 0 or 2 hours, followed by quantification of intact conjugates and released dexamethasone or calcitriol using UPLC as described above (FIG. 7A, FIG. 7B and FIG. 7C). Various levels of intact ricinoleyl-drug conjugate were detected, indicating differential levels of breakdown by plasma esterases (FIG. 7A).

Notably, the various amounts of free dexamethasone that were detected in mouse plasma corresponded to the levels of breakdown exhibited by the pro-drugs in FIG. 7B.

Dexamethasone is known to suppress unwanted immune responses. The activity of ricinoleyl-based conjugates in a cellular model of immune stimulation mediated by lipopolysaccharide (LPS) was next demonstrated.

Cultured macrophage cell lines J774.2 (FIG. 8) and Raw264.7 (FIG. 9A) were incubated with immunostimulant LPS and LNP with or without ricinoleyl-dexamethasone conjugate INT-D034/INT-D035. After 24 hours, cells were harvested and were analyzed for expression of pro-Inflammatory cytokines IL1β, TNFα and IL-6. RNA was isolated from the cells and levels of pro-inflammatory cytokines ILIβ, TNFα and IL6 were determined by qRT-PCR.

Cells incubated with a control formulation (i.e. without ricinoleyl-dexamethasone conjugate) showed elevated levels of all 3 cytokines suggesting an inflammatory response. In contrast, cells treated with LNP formulation containing either INT-D034 or INT-D035 showed reduced levels of pro-inflammatory cytokines in a dose-dependent manner. Similar reductions in ILIA levels were observed for INT-D045, INT-D046, INT-D047, INT-D048 and INT-D049 in Raw264.7 (FIG. 9B). These results suggest that ricinoleyl-dexamethasone conjugates can be processed intracellularly to release active drug (dexamethasone) to suppress an unwanted immune response.

Dexamethasone and calcitriol can tolerize antigen presenting cells (APCs). The activity of ricinoleyl-based dexamethasone and calcitriol conjugates was next demonstrated in a mixed lymphocyte reaction (MLR) assay for evaluation of immune tolerance. Bone marrow derived dendritic cells (BMDCs) from C57BI/6 male mice (Charles River) were first treated with LNP containing various mol % of dexamethasone or calcitriol conjugates for 48 hours and then activated by incubation with LPS for 24 hours. They were then harvested and mixed with CD4+ T cells isolated from Balb/cl male mice (Jackson Laboratories) at 5:1 or 10:1 T-to-BMDC ratio. The levels of T cells proliferation after 3 days were quantified using flow cytometry. As shown in FIG. 10, LNP containing 10-99 mol % of dexamethasone conjugates (INT-D034 or INT-D045) or calcitriol conjugates (INT-D053 or INT-D083) were able to suppress allogeneic T-cell proliferation, indicating that these ricinoleyl-based conjugates can be processed intracellularly to release dexamethasone or calcitriol to tolerize BMDCs.

Thus, the pro-drugs described herein can not only be loaded efficiently at large amounts into LNPs to enable controlled drug release, but are also active as shown in an in vitro model of immune stimulation and ex vivo model of immune tolerance.

### Example 7: Additional Pro-Drug Examples

Various classes of drugs can be used as the pro-drugs described herein. Select examples of such compounds are shown below and include acetylsalicylic acid, MCC950, INT-MA014, calcitriol, ruxolitinib, tofacitinib, sirolimus, docetaxel, mycophenolic acid, cannabidiol and tetrahydrocannabinol. Exemplary pro-drugs of such compounds are also depicted below:

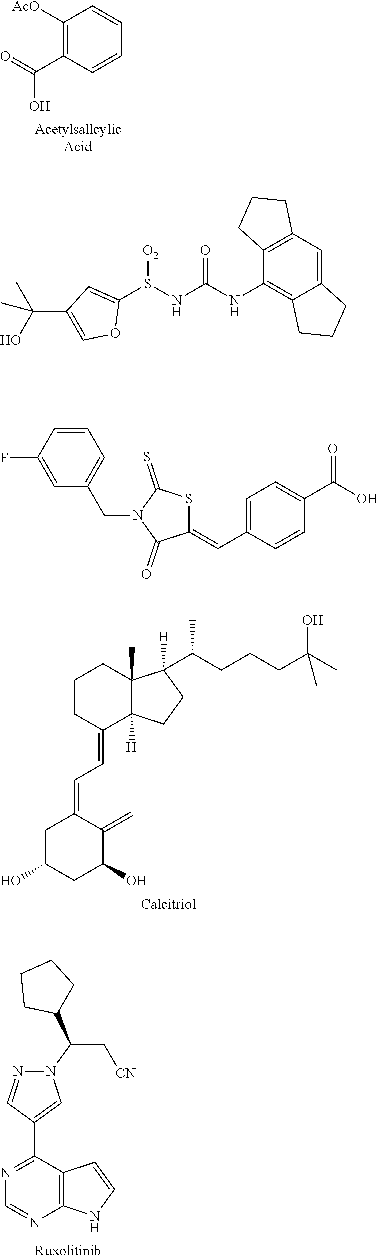

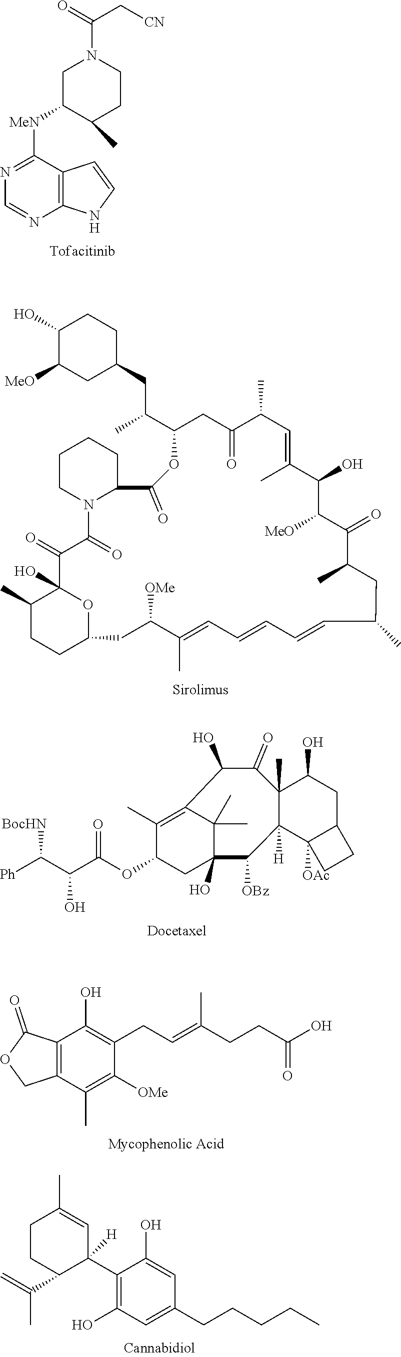

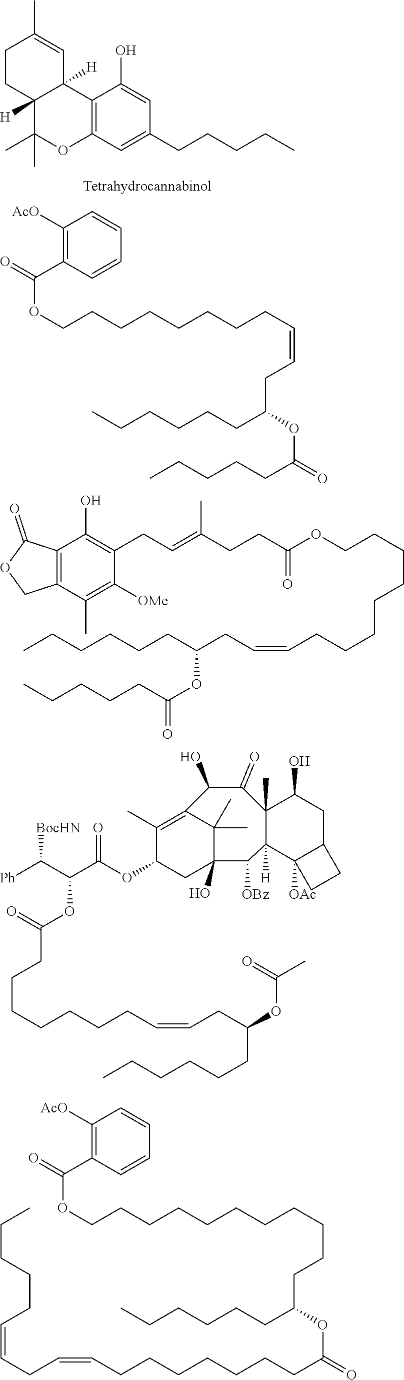

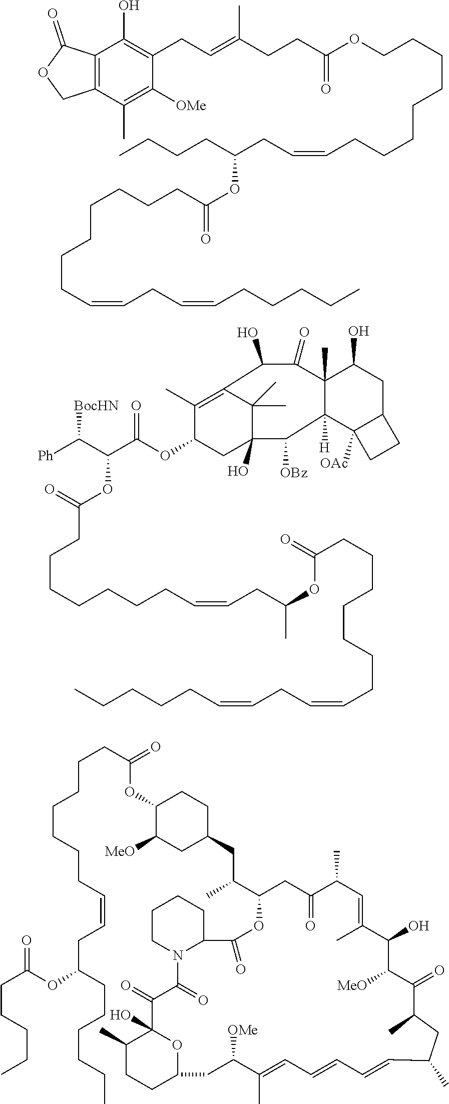

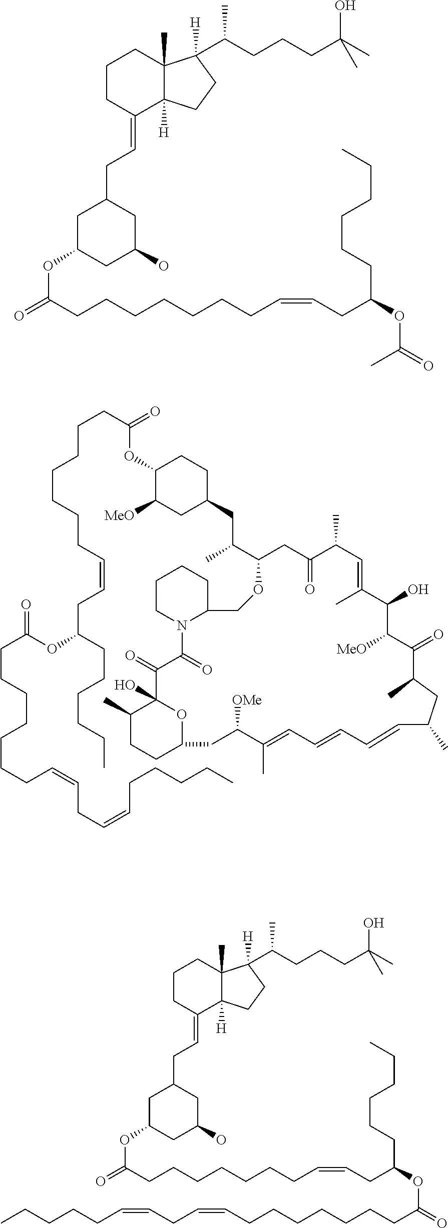

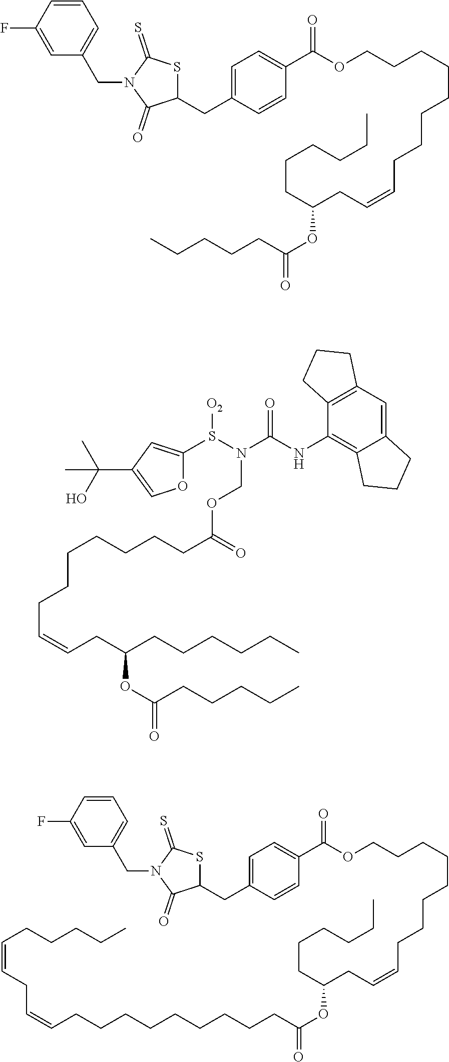

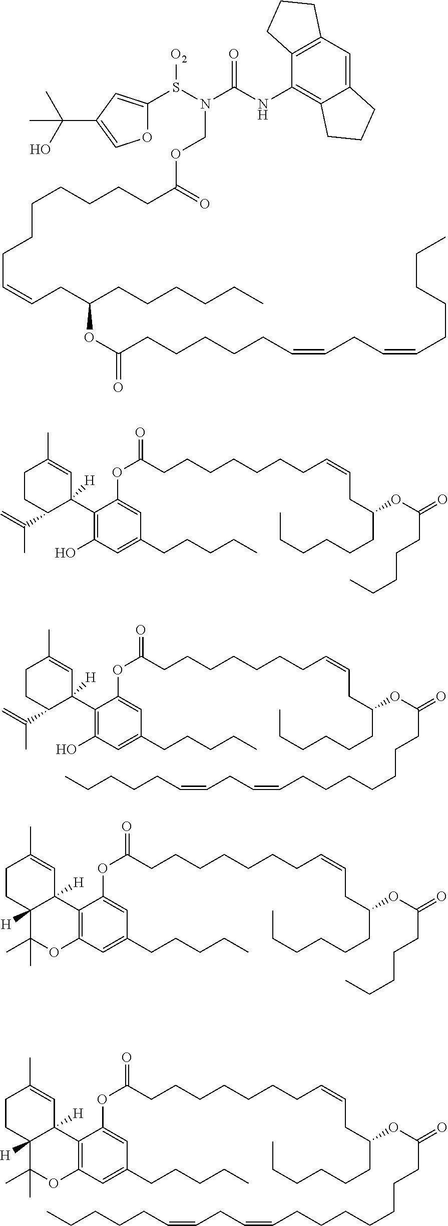

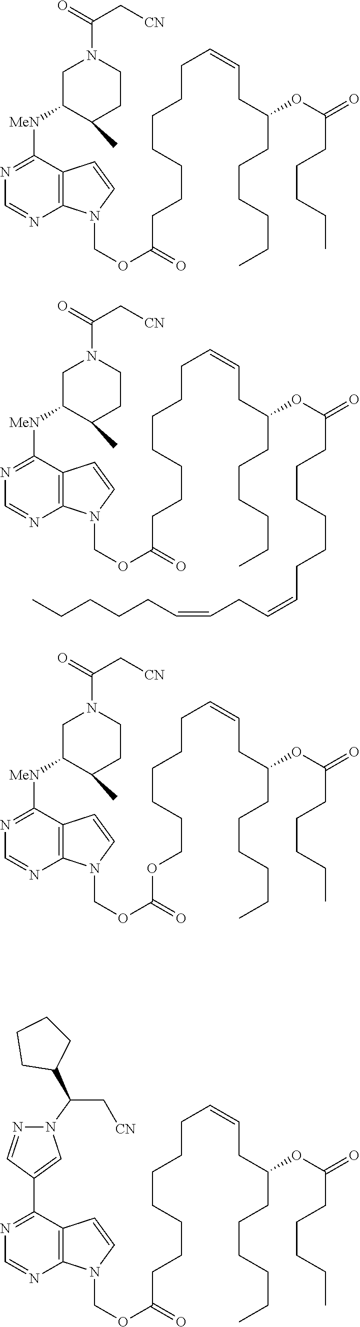

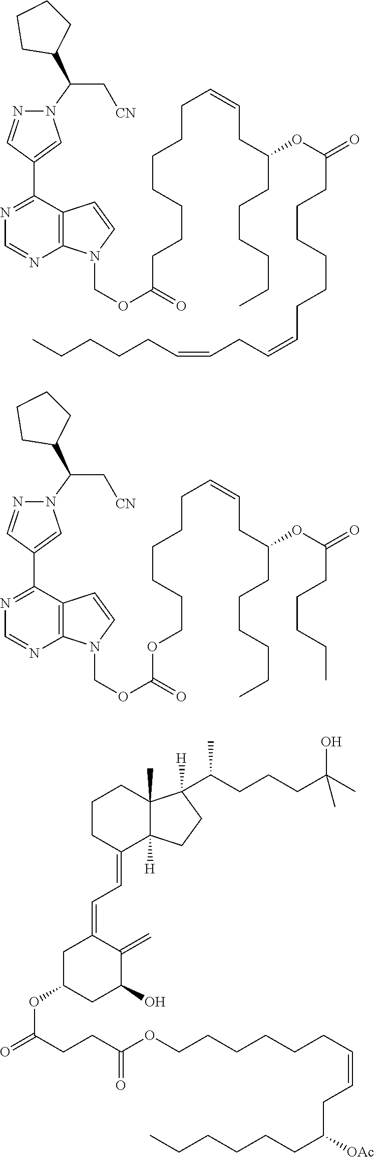

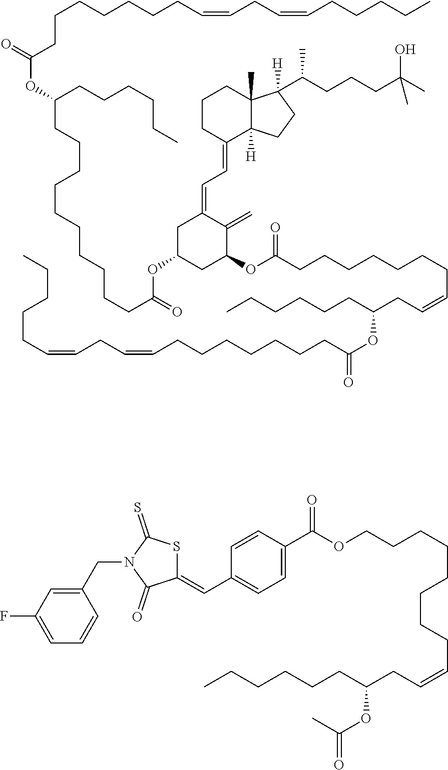 [SMILES: C=C1/C(=C\C=C2/CCC[C@]3(C)[C@@H]([C@H](C)CCCC(C)(C)O)CC[C@@H]23)C[C@@H](OC(=O)CCCCCCCCCC[C@@H](CCCCCC)OC(=O)CCCCCCC/C=C\C/C=C\CCCCC)C[C@@H]1OC(=O)CCCCCCC/C=C\C[C@@H](CCCCCC)OC(=O)CCCCCCC/C=C\C/C=C\CCCCC ; CCCCCC[C@H](C/C=C\CCCCCCCCOC(=O)c1ccc(/C=C2\SC(=S)N(Cc3cccc(F)c3)C2=O)cc1)OC(C)=O]

These pro-drugs may be synthesized using ester or carbonate X1 linker groups as shown in the reaction schemes below. The mechanism of biodegradation of the ruxolitinib pro-drug having an ester X1 linkage is also depicted below, in a first step, an esterase cleaves the ester group on the pro-drug. This is followed by spontaneous decomposition of the resulting hemiaminal to liberate the free drug.

Exemplary Syntheses of Ruxolitinib Prodrugs Using Ester and Carbonate:

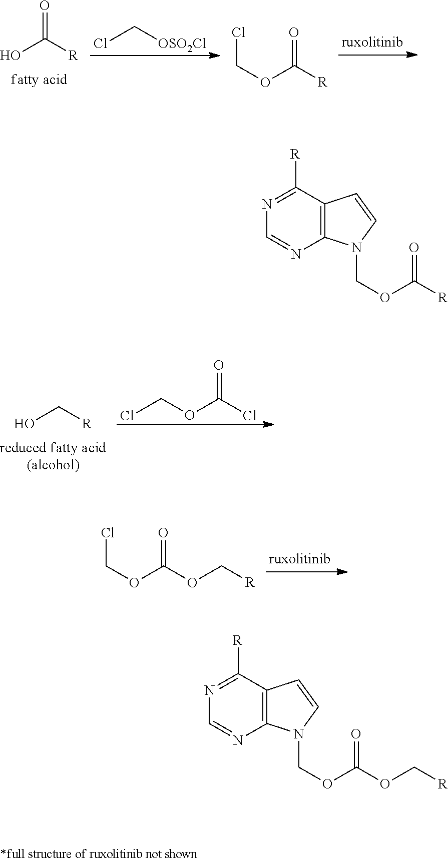

Mechanism of Biodegradation:

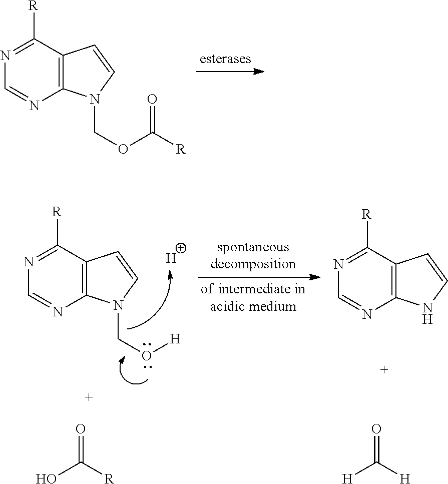

### Example 8—More than One Pro-Drug can be Formulated in the Same LNP

As mentioned above, the lipid-like properties of the pro-drugs enable ease of loading in LNP systems by simply mixing them with the lipid formulation components. It was determined that one or more pro-drugs from different respective parent drugs can be loaded in the same LNP system as these pro-drugs bear lipid-like properties. Table 7 shows LNP formulations produced by mixing two different pro-drugs at equimolar ratio (i.e., 10 mol % each). In particular, it was demonstrated that pro-drugs of dexamethasone and calcitriol could be encapsulated together at very high levels (close to 100%) to produce monodispersed nanoparticle formulations of 44-50 nm in diameter with PDI<0.1. Electron micrographs in FIG. 11 show that these combination formulations exhibit globular electron-dense area immediately at the membrane, similar to what was observed in formulations containing a single pro-drug as seen in FIGS. 4 and 5. Without being limiting, these morphological data suggest that pro-drugs of different parent drug may co-exist as a hydrophobic oil phase in the LNP lipid bilayer. In addition, it was determined that various ratios of dexamethasone and calcitriol conjugates (ranging from 1-10 mol % each) can be formulated together at high encapsulation efficiencies to form monodispersed nanoparticles of ˜50-60 nm in diameter (Table 8). LNP formulations containing 10 mol % of dexamethasone conjugate (INT-D045) with or without 10 mol % of calcitriol conjugate (INT-053, INT-D068 or INT-D083) were subjected to incubation in human plasma or more plasma for 0, 2 or 24 hours at 3° C. to determine lipid-conjugate dissociation and biodegradation as described in Example 5 (FIG. 12 and FIG. 13). Combination formulations (i.e. formulations containing more than one lipid-drug conjugates) showed similar levels of lipid dissociation or biodegradation as compared to formulations containing only one lipid-drug conjugate. These data indicate that a certain lipid-drug conjugate can remain functional when encapsulated with another lipid-drug conjugate in the same lipid nanoparticle.

TABLE 7
Particle size and polydispersity index of LNP
containing combinations of pro-drugs

| | | | | Compound | Compound |
|---|---|---|---|---|---|
| | | | Poly- | #1 Encap- | #2 Encap- |
| | | Particle | dispersity | sulation | sulation |
| Compound | Compound | Diameter | index | Efficiency | Efficiency |
| #1 | #2 | (nm) | (PDI) | (%) | (%) |
| INT-D034 | INT-D053 | 50 | 0.058 | 98 | 97 |
| INT-D034 | INT-D083 | 50 | 0.044 | 98 | 96 |
| INT-D045 | INT-D053 | 48 | 0.059 | 100 | 100 |
| INT-D045 | INT-D068 | 44 | 0.065 | 98 | 94 |
| INT-D045 | INT-D083 | 49 | 0.043 | 100 | 100 |

TABLE 8
Particle size and polydispersity index of LNP containing
different molar ratios of pro-drugs combinations

| | | | Particle | | Compound #1 | Compound #2 |
|---|---|---|---|---|---|---|
| Compound | Compound | Molar ratio | Diameter | Polydispersity | Encapsulation | Encapsulation |
| #1 | #2 | (#1:#2) | (nm) | index (PDI) | Efficiency (%) | Efficiency (%) |
| INT-D045 | INT-D053 | 10:10 | 51 | 0.051 | 100 | 100 |
| | | 3:10 | 53 | 0.058 | 100 | 97 |
| | | 1:10 | 51 | 0.065 | 93 | 97 |
| | | 10:3 | 49 | 0.049 | 100 | 98 |
| | | 10:1 | 48 | 0.037 | 99 | 94 |
| | | 3:3 | 52 | 0.069 | 94 | 98 |
| | | 1:1 | 56 | 0.047 | 100 | 97 |
| INT-D045 | INT-D068 | 10:10 | 47 | 0.055 | 99 | 98 |
| | | 3:10 | 45 | 0.059 | 94 | 95 |
| | | 1:10 | 44 | 0.054 | 95 | 98 |
| | | 10:3 | 48 | 0.054 | 99 | 96 |
| | | 10:1 | 47 | 0.055 | 99 | 74 |
| | | 3:3 | 46 | 0.107 | 95 | 86 |
| | | 1:1 | 51 | 0.035 | 95 | 86 |
| INT-0045 | INT-D083 | 10:10 | 53 | 0.034 | 98 | 98 |
| | | 3:10 | 55 | 0.046 | 85 | 97 |
| | | 1:10 | 55 | 0.050 | 100 | 98 |
| | | 10:3 | 51 | 0.044 | 97 | 91 |
| | | 10:1 | 48 | 0.055 | 96 | 97 |
| | | 3:3 | 54 | 0.049 | 97 | 83 |
| | | 1:1 | 59 | 0.033 | 100 | 76 |

The foregoing examples are illustrative only. That is, various alterations can be made without departing from the scope of certain aspects of the invention as described herein.

1-25. (canceled)

26. A lipid-conjugate comprising a branched lipid moiety having a backbone L that is a scaffold for linkage of one or more R hydrocarbon chains thereto, the lipid moiety having the structure of Formula IId: 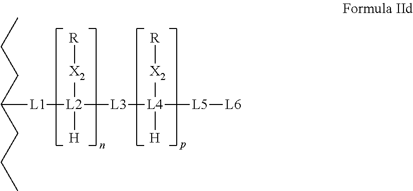
wherein a terminal portion of L1 is chemically linked to a molecule of interest (M) either directly or via a linker region having one or more heteroatoms, or wherein the terminal end of L1 comprises a linker having heteroatoms and the molecule of interest (M) is associated with the heteroatoms of such linker by hydrogen bonds;
wherein the L lipid scaffold carbon backbone is represented by L1+L2+L3+L4+L5+L6 and wherein L comprises 8 to 40 carbon atoms and 0 to 2 cis or trans C═C double bonds;
wherein L1 is a linear hydrocarbon chain having 5 to 30 carbon atoms and optionally L1 0 to 2 cis or trans C═C double bonds;
wherein L2 and L4 are each carbon atoms and wherein the L2, L4 or both L2 and L4 carbon atoms are branch points for a respective hydrocarbon R linked via respective X2 groups;
L3 is a linear hydrocarbon chain having 0 to 20 carbon atoms and comprises 0 to 2 cis or trans C═C double bonds;
L5 is a linear hydrocarbon chain having up to 20 carbon atoms and comprises 0 to 2 cis or trans C═C double bonds;
L6 is —CH₃, or ═CH₂;
wherein the L2+L3+L4+L5+L6 portion of the L lipid scaffold carbon backbone has at least 3 carbon atoms;
each R is independently a linear or branched hydrocarbon chain having 2 to 30 carbon atoms and 0 to 2 cis or trans C═C double bonds, wherein if one or more of R is branched, each branch point includes an X2 functional group;
wherein n+p is 1 to 8;
wherein n is >1;
wherein each X2 is independently an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo or urea; and
wherein the conjugate is not an ionizable lipid.

27. The conjugate of claim 26, wherein X2 is independently a group that is biodegradable post-administration to a patient.

28. The conjugate of claim 26, wherein X2 is independently a carbamate, ether or ester linkage.

29. The conjugate of claim 26, wherein L is linked to the molecule of interest M in the conjugate at L1 by an X1 to form M-X1-L, wherein X1 is an ester, amide, amidine, hydrazone, ether, carbonate, carbamate, thionocarbamate, guanidine, guanine, oxime, isourea, acylsulfonamide, phosphoramide, phosphonamide, phosphoramidate, phosphate, phosphonate, phosphodiester, phosphate phosphonooxymethylether, N-Mannich adduct, N-acyloxyalkylamine, sulfonamide, imine, azo or urea.

30. The conjugate of claim 26, wherein L is linked to the molecule of interest M in the conjugate at L1 by an X1 to form M-X1-L, wherein X1 comprises a carbon-based functional group selected from an alkane, alkene or alkyne.

31. The conjugate of claim 29, wherein X1 is an ester, ether or carbamate.

32. The conjugate of claim 26, wherein L1 has between 5 and 25 carbon atoms.

33. The conjugate of claim 26, wherein L1 has between 5 and 20 carbon atoms.

34. The conjugate of claim 26, wherein R is branched and each branch point is independently selected from an ester, ether or carbamate.

35. A method for preparing the conjugate of claim 26, the method comprising:
providing a lipid moiety that is derived from a lipid having one or more reactive groups selected from a hydroxyl and/or an amino bonded to an internal carbon atom thereof, wherein the lipid moiety serves as the backbone L that is the scaffold for linkage of the one or more R hydrocarbon chains thereto; and
reacting at least one acyl lipid comprising the R hydrocarbon chain or an acylating agent comprising the R hydrocarbon chain of Formula IId with the one or more reactive groups, thereby forming the X2-R moiety or moieties linked to L2, L4 or both L2 and L4 of Formula IId.

36. The conjugate of claim 29, wherein p of Formula IId is >1.

37. The conjugate of claim 36, wherein the R of L4-X2-R is branched and has a structure of Formula IId.

38. A lipid-conjugate comprising a branched lipid moiety having a backbone L that is a scaffold for linkage of one or more R hydrocarbon chains thereto, the lipid moiety having the structure of Formula IIe: 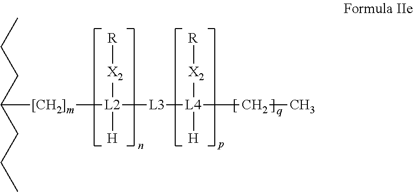
wherein a terminal portion of [CH₂]ₘ is chemically linked to a molecular of interest (M);
wherein L is denoted by [CH₂]ₘ-L2-L3-L4-[CH₂]_{q}-CH₃, wherein the total number of carbon atoms in L is 8 to 30;
L2 and L4 are carbon atoms, and wherein the L2, L4 or both L2 and L4 carbon atoms are branch points for a respective hydrocarbon R linked via respective X2 groups;
wherein m is 5 to 20; n is 1 to 4, p is 0 to 4, and n+p is 1 to 4; L3 is a linear hydrocarbon chain and has 0 to 10 carbon atoms and has 0 to 2 cis or trans C═C;
X2 are independently selected from an ether, ester and carbamate group;
wherein q is an integer and the L3-L4-[CH₂]_{q}— CH₃ portion of the L lipid scaffold carbon backbone has at least 3 carbon atoms;
wherein each R is independently:
(a) a linear or branched terminating hydrocarbon chain with 0 to 5 cis or trans C═C and 1 to 30 carbon atoms and wherein each R is conjugated to one of a respective X2 at any carbon atom in its hydrocarbon chain thereof, or
(b) a branched structure of Formula IIb having a scaffold denoted by L′: 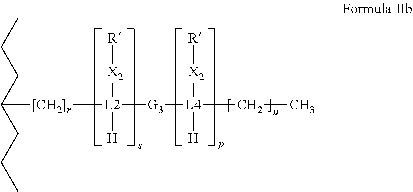
wherein L′ is denoted by [CH₂]ᵣ-L2-G₃-L4-[CH₂]ᵤ—CH₃, wherein the total number of carbon atoms in L is 3 to 30;
wherein r is 0 to 20, 2 to 20, 3 to 20 or 4 to 20;
s is 0 to 4, t is 0 to 4; and wherein s+t is >1 or is 1 to 4;
u is 1 to 20;
G₃ is 0 to 10 carbon atoms and has 0 to 2 cis or trans C═C;
wherein each R′ of Formula IIb is independently a linear or branched terminating hydrocarbon chain with 0 to 5 cis or trans C═C and 1 to 30 carbon atoms;
wherein the total number of R′ hydrocarbon chains in Formula IIb is 1 to 16;
wherein each one of the R and R′ hydrocarbon chains in the lipid moiety is optionally substituted with a heteroatom, with the proviso that no more than 8 heteroatoms are substituted in the R and R′ hydrocarbon chains and wherein the predicted or experimental log P of the conjugate is greater than 5; and
wherein the lipid-conjugate is not an ionisable lipid.

39. A pharmaceutical, nutritional, cosmetic, cleaning or foodstuff product comprising the conjugate of claim 26.

40. A nanoparticle comprising the conjugate of claim 26.

41. The nanoparticle of claim 40, wherein the nanoparticle is a lipid nanoparticle.

42. The nanoparticle of claim 40, wherein the nanoparticle comprises one or more bilayers.

43. The nanoparticle of claim 40, wherein the encapsulation efficiency of the lipid conjugate is at least 80% and the polydispersity (PDI) of the nanoparticle is less than 0.15.

44. The nanoparticle of claim 40, wherein the lipid conjugate exists in a hydrophobic oil phase in the nanoparticle or as a globular electron-dense at a membrane of the nanoparticle.

45. The nanoparticle of claim 40, wherein the percentage of lipid conjugate that remains with the nanoparticle after 2 hours of incubation in human serum in vitro is between 30 and 100 mol %.

46. The nanoparticle of claim 40, wherein the percentage of lipid conjugate that remains with the nanoparticle after 2 hours of incubation in human serum in vitro is between 60 and 100 mol %.

47. The nanoparticle of claim 40, wherein the percentage of lipid conjugate that remains with the nanoparticle after 2 hours of incubation in human serum in vitro is between 80 and 100 mol %.

48. The nanoparticle of claim 40, wherein the nanoparticle further comprises an additional lipid conjugate comprising a second molecule of interest M.
